SEQUENCE LISTING (8 sequences)

SEQ ID NO: 1 - DNA, 36 bp, Artificial
  Recognition sequence 1111′
aaagtctaatccgtccctgcctctatatctccactc

SEQ ID NO: 2 - DNA, 36 bp, Artificial
  Recognition sequence 1111′′
cacatttacagacctcaacctacctccaactctcac

SEQ ID NO: 3 - DNA, 88 bp, Artificial
  Amplification template 1113′
atctatctatctatctatctatctatctatctatctatctatcttctgagtggagatatagaggcagggacggattagactttttttt

SEQ ID NO: 4 - DNA, 88 bp, Artificial
  Amplification template 1113′′
gtctgtctgtctgtctgtctgtctgtctgtctgtctgtctgtcttctgtgagagttggaggtaggttgaggtctgtaaatgtgttttt

SEQ ID NO: 5 - DNA, 36 bp, Artificial; identical to SEQ ID NO: 1
  Trigger oligonucleotide

SEQ ID NO: 6 - DNA, 36 bp, Artificial
  Trigger oligonucleotide
gcattctttcttgaggagggcagcaaacgggaagag

SEQ ID NO: 7 - DNA, 36 bp, Artificial
  Trigger oligonucleotide
cacttcatatcactcactcccaatctctatctaccc

SEQ ID NO: 8 - DNA, 36 bp, Artificial; identical to SEQ ID NO: 2
  Trigger oligonucleotide

(19) United States
(12) Patent Application Publication
(10) Pub. No.: US 2022/0356514 A1
(43) Pub. Date: Nov. 10, 2022

(54) SYSTEMS AND METHODS FOR DETECTING MULTIPLE ANALYTES

(71) Applicants: ILLUMINA, INC., San Diego, CA (US); ILLUMINA SINGAPORE PTE. LTD., Singapore (SG); ILLUMINA CAMBRIDGE LIMITED, Cambridge (GB)

(72) Inventors: Sarah SHULTZABERGER, San Diego, CA (US); Jeffrey BRODIN, San Diego, CA (US); Yin Nah TEO, Singapore (SG); Suzanne ROHRBACK, La Jolla, CA (US); Rebecca MACLEOD, San Diego, CA (US); Rigo PANTOJA, San Diego, CA (US); Allen ECKHARDT, San Diego, CA (US); Jeffrey FISHER, San Diego, CA (US); Xiangyuan YANG, Singapore (SG); Kaitlin PUGLIESE, San Diego, CA (US); Misha GOLYNSKIY, San Diego, CA (US); Xiaolin WU, Cambridge (GB); Seth MCDONALD, San Diego, CA (US)

(73) Assignees: ILLUMINA, INC., San Diego, CA (US); ILLUMINA SINGAPORE PTE. LTD., Signapore (SG); ILLUMINA CAMBRIDGE LIMITED, Cambridge (GB)

(21) Appl. No.: 17/761,972

(22) PCT Filed: Oct. 12, 2020
(86) PCT No.: PCT/EP2020/078653

§ 371 Date: Mar. 18, 2022

Related U.S. Application Data
(60) Provisional application No. 62/916,073, filed on Oct. 16, 2019.
(60) Provisional application No. 63/014,905, filed on Apr. 24, 2020.
(60) Provisional application No. 63/014,913, filed on Apr. 24, 2020.

(51) Int. Cl. C12Q 1/6837; C12Q 1/682

(52) U.S. Cl. CPC C12Q 1/6837; C12Q 1/682

(57) ABSTRACT

A method for detecting different analytes includes mixing different analytes with sensing probes, wherein at least some of the sensing probes are specific to respective ones of the analytes. The analytes respectively are captured by the sensing probes that are specific to those analytes. Fluorophores respectively are coupled to sensing probes that captured respective analytes. The sensing probes are mixed with beads, wherein the beads are specific to respective ones of the sensing probes, and wherein the beads include different codes identifying the analytes to which those sensing probes are specific. The sensing probes respectively are coupled to beads that are specific to those sensing probes. The beads are identified that are coupled to the sensing probes that captured analytes using at least fluorescence from the fluorophores coupled to those sensing probes. The analytes that are captured are identified.

### CROSS REFERENCE TO RELATED APPLICATIONS

This application is a U.S. national stage filing under 35 U.S.C. § 371 of International Application No. PCT/EP2020/078653 entitled “Systems and Methods for Detecting Multiple Analytes,” filed Oct. 12, 2020, the entire contents of which are incorporated by reference herein, which claims the benefit of the following applications:

U.S. Provisional Patent Application No. 62/916,073, filed on Oct. 16, 2019 and entitled “Methods and Compositions for the Enrichment and Detection of Nucleic Acids,” the entire contents of which are incorporated by reference herein.

U.S. Provisional Patent Application No. 63/014,913, filed on Apr. 24, 2020 and entitled “Bead-Based System for Optically Detecting Multiple Analytes,” the entire contents of which are incorporated by reference herein.

U.S. Provisional Patent Application No. 63/014,905, filed on Apr. 24, 2020 and entitled “Amplifying Optical Detection of Analytes Using Multiple Fluorophores,” the entire contents of which are incorporated by reference herein.

### SEQUENCE LISTING

The instant application contains a Sequence Listing which has been submitted electronically in ASCII format and is hereby incorporated by reference in its entirety. Said ASCII copy, created on Mar. 17, 2022, is named IP-1980-US_SL.txt and is 2,113 bytes in size.

### BACKGROUND

The detection of specific nucleic acid sequences present in a biological sample has been used, for example, as a method for identifying and classifying microorganisms, diagnosing infectious diseases, detecting and characterizing genetic abnormalities, identifying genetic changes associated with cancer, studying genetic susceptibility to diseases, and measuring response to various types of treatment. A common technique for detecting specific nucleic acid sequences in a biological sample is nucleic acid sequencing.

Nucleic acid sequencing methodology has evolved from the chemical degradation methods used by Maxam and Gilbert and the strand elongation methods used by Sanger. Several sequencing methodologies are now in use which allow for the parallel processing of thousands of nucleic acids all on a single chip. Some platforms include bead-based and microarray formats in which silica beads are functionalized with probes depending on the application of such formats in applications including sequencing, genotyping, or gene expression profiling.

Some sequencing systems use fluorescence-based detection, whether for “sequencing-by-synthesis” or for genotyping, in which a given nucleotide is labeled with a fluorescent label, and the nucleotide is identified based on detecting the fluorescence from that label.

### SUMMARY

In some examples provided herein is a method for detecting different analytes. The method may include mixing different analytes with sensing probes, wherein at least some of the sensing probes are specific to respective ones of the analytes. The method may include respectively capturing the analytes by the sensing probes that are specific to those analytes. The method may include respectively coupling fluorophores to sensing probes that captured respective analytes. The method may include mixing the sensing probes with beads, wherein the beads are specific to respective ones of the sensing probes, and wherein the beads include different codes identifying the analytes to which those sensing probes are specific. The method may include respectively coupling the sensing probes to beads that are specific to those sensing probes. The method may include identifying the beads that are coupled to the sensing probes that captured analytes using at least fluorescence from the fluorophores coupled to those sensing probes. The method may include identifying the analytes that are captured by the sensing probes coupled to the identified beads using at least the codes of those beads.

In some examples, each of the beads includes a first oligonucleotide having a sequence specific to one of the sensing probes, and wherein each of the sensing probes includes a second oligonucleotide having a sequence that is complementary to the first oligonucleotide. In some examples, the different codes include oligonucleotides having different sequences than one another.

In some examples, at least one of the analytes includes a nucleotide analyte. In some examples, the sensing probe includes an oligonucleotide sequence specific to hybridize to the nucleotide analyte. In some examples, the nucleotide analyte includes a DNA analyte. In some examples, the nucleotide analyte includes an RNA analyte.

In some examples, at least one of the analytes includes a non-nucleotide analyte. In some examples, the non-nucleotide analyte includes a protein. In some examples, the non-nucleotide analyte includes a metabolite. In some examples, the sensing probe includes an antibody selective to the non-nucleotide analyte. In some examples, the sensing probe includes an aptamer selective to the non-nucleotide analyte.

In some examples, the different analytes include a plurality of nucleotide analytes and a plurality of non-nucleotide analytes.

In some examples, the fluorophores are coupled to the sensing probes after the analytes are captured by the sensing probes. In some examples, the fluorophores are coupled to the sensing probes before the sensing probes are coupled to the beads. In some examples, the fluorophores are coupled to the sensing probes after the sensing probes are coupled to the beads. In some examples, providing the fluorophores includes coupling multiple fluorophores to the analytes. In some examples, coupling multiple fluorophores to the analytes includes using a hybridization chain reaction (HCR).

In some examples provided herein is a system for detecting a plurality of different analytes. The system may include sensing probes that are specific to, and can capture, respective ones of the different analytes. The system may include beads that are specific to, and can couple to, respective ones of the sensing probes and that include different codes respectively identifying the analytes to which those sensing probes are specific. The system may include fluorophores to respectively couple to sensing probes that capture analytes. The system may include detection circuitry to identify beads that are coupled to the sensing probes that captured analytes, and to identify the analytes that are captured by the sensing probes coupled to those beads using at least the codes of those beads.

In some examples, each of the beads includes a first oligonucleotide having a sequence specific to one of the sensing probes, and each of the sensing probes includes a second oligonucleotide having a sequence that is complementary to the first oligonucleotide. In some examples, the different codes include oligonucleotides having different sequences than one another.

In some examples, at least one of the analytes includes a nucleotide analyte. In some examples, the sensing probe includes an oligonucleotide sequence specific to hybridize to the nucleotide analyte. In some examples, the nucleotide analyte includes a DNA analyte. In some examples, the nucleotide analyte includes an RNA analyte.

In some examples, at least one of the analytes includes a non-nucleotide analyte. In some examples, the non-nucleotide analyte includes a protein. In some examples, the non-nucleotide analyte includes a metabolite. In some examples, the sensing probe includes an antibody selective to the non-nucleotide analyte. In some examples, the sensing probe includes an aptamer selective to the non-nucleotide analyte.

In some examples, the different analytes include a plurality of nucleotide analytes and a plurality of non-nucleotide analytes.

In some examples, the fluorophores are coupled to the sensing probes after the analytes are captured by the sensing probes. In some examples, the fluorophores are coupled to the sensing probes before the sensing probes are coupled to the beads. In some examples, the fluorophores are coupled to the sensing probes after the sensing probes are coupled to the beads. In some examples, multiple fluorophores are coupled to the analytes.

In some examples, the multiple fluorophores are coupled to the analytes using a hybridization chain reaction (HCR).

Some examples of the methods and compositions provided herein include a method for identifying target nucleic acids, comprising: (a) hybridizing a plurality of probes to a plurality of nucleic acids comprising the target nucleic acids, wherein each probe comprises a 3′ end capable of hybridizing to a target nucleic acid and a 5′ end capable of hybridizing to a capture probe; (b) extending the hybridized probes with a blocked nucleotide; (c) removing the plurality of nucleic acids and non-extended probes from the extended probes; and (d) hybridizing the extended probes to a plurality of capture probes immobilized on a surface. In some examples, (a)-(c) are performed in solution.

Some examples also include repeating (a) and (b).

In some examples, the blocked nucleotide comprises a detectable label. In some examples, the label comprises a fluorophore.

In some examples, (b) comprises polymerase extension. In some examples, (b) comprises ligase extension.

In some examples, (c) comprises enzymatic degradation. In some examples, (c) comprises contacting the plurality of nucleic acids and the non-extended probes with a 3′ to 5′ exonuclease. In some examples, the 3′ to 5′ exonuclease is selected from the group consisting of Exonuclease I, Thermolabile Exonuclease I, Exonuclease T, Exonuclease III, and Klenow I fragment.

In some examples, the probes each comprise a 5′ end resistant to enzymatic degradation. In some examples, the 5′ end resistant to enzymatic degradation comprises a phosphorothioate bond. In some examples, (c) comprises contacting the plurality of nucleic acids with a 5′ to 3′ exonuclease. In some examples, the 5′ to 3′ exonuclease is selected from the group consisting of RecJf, T7 Exonuclease, truncated Exonuclease VIII, Lambda Exonuclease, T5 Exonuclease, Exonuclease VII, Exonuclease V, and Nuclease BAL-31.

In some examples, a plurality of beads comprise the surface.

In some examples, the surface comprises a planar surface.

In some examples, a flow cell comprises the surface.

In some examples, (d) further comprises amplifying a signal from the hybridized extended probes.

In some examples, (d) further comprises identifying the location of the hybridized extended probes on the surface.

In some examples, the capture probes are different from each other.

In some examples, the plurality of capture probes comprise a decoded array of capture probes. Some examples also include decoding the location of the capture probes on the surface. In some examples, the plurality of capture probes each comprise a primer binding site and a decode polynucleotide. In some examples, decoding comprises: hybridizing a sequencing primer to the primer binding site, extending the hybridized primer, and identifying the decode polynucleotide.

In some examples, the plurality of nucleic acids comprises genomic DNA. In some examples, the target nucleic acids comprise a single nucleotide polymorphism (SNP).

Some examples of the methods and compositions provided herein include a system for identifying target nucleic acids, comprising: an extension solution comprising: a plurality of nucleic acids comprising the target nucleic acids, a plurality of probes, wherein each probe comprises a 3′ end capable of hybridizing to a target nucleic acid and a 5′ end capable of hybridizing to a capture probe, a plurality of blocked nucleotides, an extension enzyme; a degradation solution comprising a 3′ to 5′ exonuclease; an array of capture probes immobilized on a surface; and a detector to identify the location of an extended probe hybridized to a capture probe on the surface. In some examples, a flow cell comprise the array of capture probes immobilized on a surface.

Some examples of the methods and compositions provided herein include a system for identifying target nucleic acids, comprising: a flow cell comprising a surface, an inlet for adding solutions to the surface, and an outlet for removing solutions from the surface, wherein an array of capture probes is immobilized on the surface; an extension solution in contact with the inlet, the extension solution comprising: a plurality of nucleic acids comprising the target nucleic acids, a plurality of probes, wherein each probe comprises a 3′ end capable of hybridizing to a target nucleic acid and a 5′ end capable of hybridizing to a capture probe, a plurality of blocked nucleotides, an extension enzyme; a degradation solution comprising a 3′ to 5′ exonuclease; and a detector to identify the location of an extended probe hybridized to a capture probe on the surface.

In some examples, the blocked nucleotide comprises a detectable label. In some examples, the label comprises a fluorophore.

In some examples, the extension enzyme comprises a polymerase. In some examples, the extension enzyme comprises a ligase.

In some examples, the 3′ to 5′ exonuclease is selected from the group consisting of Exonuclease I, Thermolabile Exonuclease I, Exonuclease T, Exonuclease III, and Klenow I fragment.

In some examples, the probes each comprise a 5′ end resistant to enzymatic degradation. In some examples, the 5′ end resistant to enzymatic degradation comprises a phosphorothioate bond. In some examples, the degradation solution further comprises a 5′ to 3′ exonuclease. In some examples, the 5′ to 3′ exonuclease is selected from the group consisting of RecJf, T7 Exonuclease, truncated Exonuclease VIII, Lambda Exonuclease, T5 Exonuclease, Exonuclease VII, Exonuclease V, and Nuclease BAL-31.

In some examples, the surface comprises a plurality of beads.

In some examples, the capture probes are different from each other.

In some examples, the plurality of capture probes comprise a decoded array of capture probes. In some examples, the plurality of capture probes each comprise a primer binding site and a decode polynucleotide.

In some examples, the plurality of nucleic acids comprises genomic DNA. In some examples, the target nucleic acids comprise a single nucleotide polymorphism (SNP).

In some examples provided herein is a method for detecting an element. The method may include coupling an element to a substrate. The method may include coupling a plurality of fluorophores to the element. The method may include detecting the element using at least fluorescence from the plurality of fluorophores.

In some examples, the element includes an analyte. In some examples, the analyte is coupled to a sensing probe. In some examples, the analyte is coupled to the substrate via the sensing probe. In some examples, the plurality of fluorophores is coupled to the element via the sensing probe. In some examples, the plurality of fluorophores is coupled to the element via the substrate.

In some examples, the plurality of fluorophores is coupled to the element before the element is coupled to the substrate. In some examples, the plurality of fluorophores is coupled to the element after the element is coupled to the substrate.

In some examples, the substrate includes a bead.

In some examples, the plurality of fluorophores is coupled to the element using rolling circle amplification. In some examples, the rolling circle amplification generates an elongated, repeated sequence, and wherein the plurality of fluorophores is coupled to respective, repeated portions of that sequence. In some examples, the fluorophores are coupled to DNA intercalators that couple to the elongated, repeated sequence. In some examples, the oligonucleotides including fluorophores and quenchers are hybridized to the repeated portions.

In some examples, the element is coupled to a trigger oligonucleotide to which a plurality of fluorescently labeled hairpins self-assemble. In some examples, the element is coupled to a trigger oligonucleotide including a first trigger sequence A′ and a second trigger sequence B′, and wherein coupling the plurality of fluorophores to the element includes contacting the trigger oligonucleotide with a plurality of first oligonucleotide hairpins and a plurality of second oligonucleotide hairpins. Each of the first oligonucleotide hairpins may include a first fluorophore, a single-stranded toehold sequence A complementary to first trigger sequence A′, a first stem sequence B complementary to second trigger sequence B′, a second stem sequence B′ that is temporarily hybridized to first stem sequence B, and a single-stranded loop sequence C′ disposed between the first stem sequence B and the second stem sequence B′. Each of the second oligonucleotide hairpins may include a second fluorophore, a single-stranded toehold sequence C complementary to single-stranded loop sequence C′, a first stem sequence B complementary to second trigger sequence B′, a second stem sequence B′ that is temporarily hybridized to first stem sequence B, and a single-stranded loop sequence A′ disposed between the first stem sequence B and the second stem sequence B′.

In some examples, responsive to hybridization of the single-stranded toehold sequence A of one of the first oligonucleotide hairpins to first trigger sequence A′ of the trigger oligonucleotide, the second stem sequence B′ of that first oligonucleotide hairpin dehybridizes from the first stem sequence B of that first oligonucleotide hairpin; the single-stranded toehold sequence C of one of the second oligonucleotide hairpins hybridizes to the single-stranded loop sequence of that first oligonucleotide hairpin; and the second stem sequence B′ of that second oligonucleotide hairpin dehybridizes from the first stem sequence B of that second oligonucleotide hairpin.

In some examples, responsive to hybridization of the single-stranded toehold sequence A of another one of the first oligonucleotide hairpins to single-stranded loop sequence A′ of that second oligonucleotide hairpin, the second stem sequence B′ of that first oligonucleotide hairpin dehybridizes from the first stem sequence B of that first oligonucleotide hairpin; the single-stranded toehold sequence C of another one of the second oligonucleotide hairpins hybridizes to the single-stranded loop sequence of that first oligonucleotide hairpin; and the second stem sequence B′ of that second oligonucleotide hairpin dehybridizes from the first stem sequence B of that second oligonucleotide hairpin.

In some examples, the element is coupled to an oligonucleotide primer. Coupling the plurality of fluorophores to the element may include hybridizing an amplification template to the oligonucleotide primer; and extending the oligonucleotide primer, using at least the amplification template, with a plurality of fluorescently labeled nucleotides to generate an extended strand including the plurality of fluorophores. In some examples, at least one of the fluorophores is different than at least one other of the fluorophores. In some examples, the method further includes dehybridizing the amplification template and forming the extended strand into a hairpin structure.

In some examples, the element is coupled to an oligonucleotide primer. Coupling the plurality of fluorophores to the element may include hybridizing an amplification template to the oligonucleotide primer; extending the oligonucleotide primer, using at least the amplification template, with a plurality of nucleotides that are respectively coupled to additional oligonucleotide primers; hybridizing additional amplification templates to the additional nucleotide primers; and extending the additional nucleotide primers, using at least the additional amplification templates, with a plurality of nucleotides that are either respectively coupled to fluorophores or are respectively coupled to further additional oligonucleotide primers. In some examples, the method further includes hybridizing further additional amplification templates to the further nucleotide primers; and extending the additional nucleotide primers, using at least the additional amplification templates, with a plurality of nucleotides that are either respectively coupled to fluorophores or are respectively coupled to still further additional oligonucleotide primers.

In some examples, the element is coupled to a DNA origami including the plurality of fluorophores. In some examples, the DNA origami includes a combination of different fluorophores. In some examples, the element is coupled to the DNA origami via copper(I)-catalyzed click reaction, strain-promoted azide-alkyne cycloaddition, hybridization of an oligonucleotide to a complementary oligonucleotide, biotin-streptavidin interaction, NTA-His-Tag interaction, or Spytag-Spycatcher interaction.

In some examples, the element is coupled to an oligonucleotide, and the oligonucleotide includes the plurality of fluorophores. In some examples, the oligonucleotide includes a hairpin. In some examples, the oligonucleotide further includes a radical scavenger.

In some examples, the element is directly coupled to a first oligonucleotide, and the first oligonucleotide is hybridized to a second oligonucleotide that includes the plurality of fluorophores.

In some examples provided herein is a method for detecting a nucleotide. The method may include adding the nucleotide to a first polynucleotide using at least a sequence of a second polynucleotide, wherein the added nucleotide includes a first moiety. The method may include coupling a label to the added nucleotide by reacting the first moiety with a second moiety of the label, wherein the label includes a plurality of fluorophores. The method may include detecting the added nucleotide using at least fluorescence from the plurality of fluorophores.

In some examples provided herein is another method for detecting a nucleotide. The method may include adding the nucleotide to a first polynucleotide using at least a sequence of a second polynucleotide, wherein the added nucleotide is coupled to a label including a plurality of fluorophores. The method may include detecting the added nucleotide using at least fluorescence from the plurality of fluorophores.

In some examples provided herein is another method for detecting a nucleotide. The method may include adding the nucleotide to a first polynucleotide using at least a sequence of a second polynucleotide, wherein the added nucleotide includes a first moiety. The method may include coupling a label to the added nucleotide by reacting the first moiety with a second moiety of the label. The method may include coupling multiple fluorophores to the coupled label. The method may include detecting the added nucleotide using at least fluorescence from the plurality of fluorophores.

In some examples provided herein is a composition. The composition may include a substrate; an oligonucleotide coupled to the substrate; a nucleotide coupled to the oligonucleotide; and a moiety coupled to the nucleotide. The composition also may include a label coupled to the moiety, wherein the label includes a plurality of fluorophores. The composition also may include detection circuitry configured to detect the nucleotide using at least fluorescence from the plurality of fluorophores.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1B schematically illustrate example components of a bead-based system for optically detecting multiple analytes.

FIG. 1C illustrates an example process flow for detecting multiple analytes in a bead-based system.

FIGS. 2A-2C schematically illustrate example hybridization-based process flows for optically detecting DNA analytes in a bead-based system.

FIG. 2D depicts an example for identifying a target nucleic acid which includes hybridization of a target-specific probe to the target genomic DNA fragment containing a single nucleotide polymorphism (SNP), single base extension of the hybridized probe with a modified nucleotide having a 3′ fluorophore, enzymatic degradation of unextended probes and genomic DNA, and hybridization of the extended probe to a capture probe immobilized on a bead in a decoded array of capture probes.

FIG. 2E depicts an example for identifying target nucleic acids, which example includes linear signal amplification by performing multiple cycles of probe hybridization and extension.

FIG. 2F depicts examples of enzymatic degradation of non-extended target-specific probes and genomic DNA, including the use of Exonuclease I, Klenow I fragment, and Exonuclease III.

FIGS. 3A-3B schematically illustrate example hybridization-based process flows for optically detecting RNA analytes in a bead-based system.

FIGS. 4A-4B schematically illustrate example antibody-based process flows for optically detecting protein analytes in a bead-based system.

FIGS. 5A-5C schematically illustrate example aptamer-based process flows for optically detecting protein or metabolite analytes in a bead-based system.

FIGS. 6A-6C schematically illustrates example schemes for optically quantifying analyte concentrations in a bead-based system.

FIGS. 7A-7D schematically illustrate example process flows for labeling an analyte with multiple fluorophores in a bead-based system.

FIGS. 8A-8C schematically illustrate example process flows for using rolling circle amplification (RCA) to label an analyte with multiple fluorophores in a bead-based system.

FIGS. 9A-9C schematically illustrate example process flows for using a hybridization chain reaction (HCR) to label an analyte with multiple fluorophores.

FIG. 10A schematically illustrates another example process flow for using a hybridization chain reaction (HCR) to label an analyte with multiple fluorophores.

FIG. 10B schematically illustrates example components that may be used in the process flow of FIG. 10A.

FIGS. 11A-11B schematically illustrate example process flows for using an amplification template to label an analyte with multiple fluorophores.

FIG. 11C schematically illustrates an example scheme for four-analyte discrimination that labels the elements with multiple fluorophores and uses an amplification template.

FIGS. 11D-11F schematically illustrate example analytes labeled with alternative multiple fluorophores using an amplification template.

FIG. 11G illustrates example sequences for use in a process flow for using an amplification template to label an analyte with multiple fluorophores.

FIG. 11H schematically illustrates an alternative example process flow for using an amplification template to label a nucleotide with multiple fluorophores.

FIGS. 11I-11J are plots illustrating example amplifications that may be obtained using the process flow of FIG. 11H.

FIG. 12 schematically illustrates an example process flow for using DNA origami to label an analyte with multiple fluorophores.

FIG. 13A schematically illustrates an example process flow for incorporating a DNA analyte labeled with a hairpin having multiple fluorophores into a polynucleotide.

FIG. 13B schematically illustrates an example process flow for incorporating a DNA analyte coupled to a first oligonucleotide into a polynucleotide, followed by hybridizing to the first oligonucleotide to a second oligonucleotide with multiple fluorophores.

FIG. 14 illustrates an example process flow for detecting an analyte using at least multiple fluorophores.

FIGS. 15A-15C schematically illustrate example process flows for detecting a nucleotide using at least multiple fluorophores.

FIG. 16A is a plot illustrating measured fluorescence from DNA analytes respectively labeled with single fluorophores.

FIG. 16B is a plot illustrating measured fluorescence from DNA analytes respectively labeled with multiple fluorophores using HCR.

FIG. 16C schematically illustrates an example process flow used to respectively label a plurality of DNA analytes with multiple fluorophores using HCR.

FIGS. 16D-16E are plots illustrating genotyping performance using at least the measured fluorescence from DNA analytes respectively labeled with multiple fluorophores using HCR.

FIG. 16F is a gel image showing a single base extension of a primer at the expected size (ddNTP-DNA 1st base) for variants of an SBS polymerase.

FIG. 16G is a plot illustrating that percent turnover of the ddNTPs, calculated via gel densitometry, is similar to that of their native counterparts.

### DETAILED DESCRIPTION

A bead-based system for optically detecting multiple analytes is provided herein. Also provided herein is amplification of optical detection of analytes using multiple fluorophores.

For example, the present application provides methods for expanding bead-based genotyping assays to support detection of multiple different analytes, i.e., “multiomic” detection. The analytes may include nucleic acids, such as DNA analytes or RNA analytes, well as analytes other than nucleic acids, such as proteins or metabolites. The present methods may employ solution-phase capture, for example by sensing probes, of any suitable combination of different analytes. Each of the different sensing probes may include, for example, a nucleic acid, antibody, or aptamer that is specific to a respective analyte. The analytes may be coupled to fluorophores, e.g., before or after the analytes are captured by respective sensing probes. After the analytes are captured, the different sensing probes may be selectively coupled to different substrates at which fluorescence from the fluorophores may be detected. The substrates may include codes based upon which the identity of the captured analyte may be read out. As such, the bead pool may generate a common signal for detection, and optionally quantification, of analytes (including any suitable combination of nucleotide analytes and non-nucleotide analytes). Such detection may provide high specificity by linking analyte capture to generation of fluorescent signal.

Additionally, the present application provides methods for amplifying optical signals from analytes. For technologies that use fluorescent labels to detect analytes, such as nucleotides, the intensity and uniformity of the fluorescence can affect the accuracy of the detection. As such, it may be desirable to provide labels that can generate significantly more fluorescence (e.g., 30 times more fluorescence) than a single fluorophore may be able to generate. Additionally, it may be desirable to provide labels that can generate a relatively consistent amount of fluorescence per analyte, e.g., per nucleotide, so as to permit quantitative determination of the relative abundance of analytes within a sample, or between samples. Accordingly, signal amplification strategies that generate relatively high signal and correspondingly low detection limits, while providing relatively high signal uniformity, are desirable. Provided herein are several example methods for using multiple fluorophores to amplify the optical detection of analytes. Such methods optionally may be utilized in conjunction with the bead-based system and methods for optically detecting multiple analytes such as described elsewhere herein. However, it will be appreciated that the present methods for amplifying optical detection using multiple fluorophores are not limited thereto, and suitably may be adapted to couple multiple fluorophores to any desired element.

Some terms used herein will be briefly explained. Then, some example compositions and example methods for amplification of optical detection of nucleotides using multiple fluorophores will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially”, “approximately”, and “about” used throughout this Specification are used to describe and account for small fluctuations, such as due to variations in processing. For example, they can refer to less than or equal to +5%, such as less than or equal to +2%, such as less than or equal to +1%, such as less than or equal to +0.5%, such as less than or equal to +0.2%, such as less than or equal to +0.1%, such as less than or equal to +0.05%.

As used herein, “analyte” is intended to mean a chemical element that is desired to be detected. An analyte may be referred to as a “target.” Analytes may include nucleotide analytes and non-nucleotide analytes. Nucleotide analytes may include one or more nucleotides. Non-nucleotide analytes may include chemical entities that are not nucleotides. An example nucleotide analyte is a DNA analyte, which includes a deoxyribonucleotide or modified deoxyribonucleotide. DNA analytes may include any DNA sequence or feature that may be of interest for detection, such as single nucleotide polymorphisms or DNA methylation. Another example nucleotide analyte is an RNA analyte, which includes a ribonucleotide or modified ribonucleotide. RNA analytes may include any RNA sequence or feature that may be of interest for detection, such as the presence or amount of mRNA or of cDNA. An example non-nucleotide analyte is a protein analyte. A protein includes a sequence of polypeptides that are folded into a structure. Another example non-nucleotide analyte is a metabolite analyte. A metabolite analyte is a chemical element that is formed or used during metabolism. Additional example analytes include. but are not limited to, carbohydrates, fatty acids, sugars (such as glucose), amino acids, nucleosides, neurotransmitters, phospholipids, and heavy metals. In the present disclosure, analytes may be detected in the context of any suitable application(s), such as analyzing a disease state, analyzing metabolic health, analyzing a microbiome, analyzing drug interaction, analyzing drug response, analyzing toxicity, or analyzing infectious disease. Illustratively, metabolites can include chemical elements that are upregulated or downregulated in response to disease. Nonlimiting examples of analytes include kinases, serine hydrolases, metalloproteases, disease-specific biomarkers such as antigens for specific diseases, and glucose.

As used herein, elements being “different” is intended to mean that one of the elements has at least one variation relative to the other element that renders the elements distinguishable one another. For example, nucleotide analytes that are different than one another may have nucleotide sequences that vary relative to another by at least one nucleotide. As another example, proteins that are different than one another may have peptide sequences that vary relative to one another by at least one peptide. As another example, metabolites may vary relative to one another by at least one chemical group. As provided herein, different analytes can be distinguished from one another using the present systems and methods. For example, nucleotide analytes varying by at least one nucleotide relative to one another can be detected and distinguished from one another. As another example, proteins having peptide sequences varying by at least one peptide relative to one another can be detected and distinguished from one another. As another example, metabolites varying by at least one chemical group relative to one another can be detected and distinguished from one another.

As used herein, the term “nucleotide” is intended to mean a molecule that includes a sugar and at least one phosphate group, and optionally also includes a nucleobase. A nucleotide that lacks a nucleobase can be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide can be a single stranded sequence of nucleotides, such as RNA or single stranded DNA, a double stranded sequence of nucleotides, such as double stranded DNA or double stranded RNA, or can include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides can include non-naturally occurring DNA, such as enantiomeric DNA. The precise sequence of nucleotides in a polynucleotide can be known or unknown. The following are example examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, “polynucleotide” and “nucleic acid”, may be used interchangeably, and can refer to a polymeric form of nucleotides of any length, such as either ribonucleotides or deoxyribonucleotides. Thus, this term includes single-, double-, or multi-stranded DNA or RNA. The term polynucleotide also refers to both double and single-stranded molecules. Examples of polynucleotides include a gene or gene fragment, genomic DNA, genomic DNA fragment, exon, intron, messenger RNA (mRNA), transfer RNA, ribosomal RNA, non-coding RNA (ncRNA) such as PIWI-interacting RNA (piRNA), small interfering RNA (siRNA), and long non-coding RNA (lncRNA), small hairpin (shRNA), small nuclear RNA (snRNA), micro RNA (miRNA), small nucleolar RNA (snoRNA) and viral RNA, ribozyme, cDNA, recombinant polynucleotide, branched polynucleotide, plasmid, vector, isolated DNA of any sequence, isolated RNA of any sequence, nucleic acid probe, primer or amplified copy of any of the foregoing. A polynucleotide can include modified nucleotides, such as methylated nucleotides and nucleotide analogs including nucleotides with non-natural bases, nucleotides with modified natural bases such as aza- or deaza-purines. In some examples, a polynucleotide can be composed of a specific sequence of four nucleotide bases: adenine (A); cytosine (C); guanine (G); and thymine (T). Uracil (U) can also be present, for example, as a natural replacement for thymine when the polynucleotide is RNA. Uracil can also be used in DNA. Thus, the term ‘sequence’ refers to the alphabetical representation of a polynucleotide or any nucleic acid molecule, including natural and non-natural bases.

As used herein, “target nucleic acid” or grammatical equivalent thereof can refer to nucleic acid molecules or sequences that it is desired to identify, sequence, analyze and/or further manipulate. In some examples, a target nucleic acid can include a single nucleotide polymorphism (SNP) to be identified. In some examples, a SNP can be identified by hybridizing a probe to the target nucleic acid, and extending the probe. In some examples, the extended probe can be detected by hybridizing the extended probe to a capture probe.

As used herein, the term “sensing probe” is intended to mean an element that can specifically capture an analyte and that can bind to a substrate. Sensing probes can be free-floating elements in a solution, e.g., can be mixed in a common solution with different analytes, and can be bound to respective substrates after capturing the analytes to which those sensing probes are specific. A sensing probe can include a “capture probe” which is intended to mean a sub-component that can specifically capture an analyte, and also can include a “code” that is specific to a substrate which has a complementary code. By “capture” it is meant to become coupled to an analyte that is in solution. By “code” it is meant a moiety (such as an oligonucleotide sequence) that is specific to bind to another moiety (such as a complementary oligonucleotide sequence). Thus, the capture probe of a sensing probe can capture an analyte in a solution, and the code of that sensing probe subsequently can bind to a code of a substrate with specificity, thus binding the analyte to the substrate with specificity.

Accordingly, in some examples, a “capture probe” can refer to a polynucleotide having sufficient complementarity to specifically hybridize to a target nucleic acid or other probe, such as an extended probe. A capture probe can function as an affinity binding molecule for isolation of a target nucleic acid or other probe from other nucleic acids and/or components in a mixture. In some examples, a target nucleic acid or other probe, such as an extended probe, can be specifically bound by a capture probe through intervening molecules. Examples of intervening molecules include linkers, adapters and other bridging nucleic acids having sufficient complementarity to specifically hybridize to both a target sequence and a capture probe.

As used herein, “hybridize” is intended to mean noncovalently attaching a first polynucleotide to a second polynucleotide along the lengths of those polynucleotides via specific hydrogen bonding pairing of nucleotide bases. The strength of the attachment between the first and second polynucleotides increases with the length and complementarity between the sequences of monomer units within those polymers. For example, the strength of the attachment between a first polynucleotide and a second polynucleotide increases with the complementarity between the sequences of nucleotides within those polynucleotides, and with the length of that complementarity. By “temporarily hybridized” it is meant that polymer sequences are hybridized to each other at a first time, and dehybridized from one another at a second time.

For example, as used herein, “hybridization”, “hybridizing” or grammatical equivalent thereof, can refer to a reaction in which one or more polynucleotides react to form a complex that is formed at least in part via hydrogen bonding between the bases of the nucleotide residues. The hydrogen bonding can occur by Watson-Crick base pairing, Hoogstein binding, or in any other sequence-specific manner. The complex can have two strands forming a duplex structure, three or more strands forming a multi-stranded complex, a single self-hybridizing strand, or any combination of thereof. The strands can also be cross-linked or otherwise joined by forces in addition to hydrogen bonding.

As used herein, a “polymerase” is intended to mean an enzyme having an active site that assembles polynucleotides by polymerizing nucleotides into polynucleotides. A polymerase can bind a primed single stranded polynucleotide template, and can sequentially add nucleotides to the growing primer to form a polynucleotide having a sequence that is complementary to that of the template.

As used herein, the term “primer” is defined as a polynucleotide having a single strand with a free 3′ OH group. A primer can also have a modification at the 5′ terminus to allow a coupling reaction or to couple the primer to another moiety. The primer length can be any number of bases long and can include a variety of non-natural nucleotides. A primer can be blocked at the 3′ end to inhibit polymerization until the block is removed.

As used herein, “extending”, “extension” or any grammatical equivalents thereof can refer to the addition of dNTPs to a primer, polynucleotide or other nucleic acid molecule by an extension enzyme such as a polymerase, or ligase.

As used herein, “ligation” or “ligating” or other grammatical equivalents thereof can refer to the joining of two nucleotide strands by a phosphodiester bond. Such a reaction can be catalyzed by a ligase. A ligase can include an enzyme that catalyzes this reaction with the hydrolysis of ATP or a similar triphosphate.

As used herein, the term “label” is intended to mean a structure that is coupled to an element and based upon which the presence of an element can be detected. A label may include a fluorophore, or may include a moiety to which a fluorophore may be coupled directly or indirectly. For example, the fluorophore may be directly to the analyte, or may be coupled indirectly to the analyte by being coupled to a sensing probe or to a bead to which the analyte is or previously was coupled.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flow cell, or a bead located in a flow cell.

Substrates can be non-patterned, textured, or patterned on one or more surfaces of the substrate. In some examples, the substrate is patterned. Such patterns may include posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, Calif.). Beads may be located in a flow cell.

As used herein, “surface” can refer to a part of a substrate or support structure that is accessible to contact with reagents, beads or analytes. The surface can be substantially flat or planar. Alternatively, the surface can be rounded or contoured. Example contours that can be included on a surface are wells, depressions, pillars, ridges, channels or the like. Example materials that can be used as a substrate or support structure include glass such as modified or functionalized glass; plastic such as acrylic, polystyrene or a copolymer of styrene and another material, polypropylene, polyethylene, polybutylene, polyurethane or TEFLON; polysaccharides or cross-linked polysaccharides such as agarose or Sepharose; nylon; nitrocellulose; resin; silica or silica-based materials including silicon and modified silicon; carbon-fibre; metal; inorganic glass; optical fibre bundle, or a variety of other polymers. A single material or mixture of several different materials can form a surface useful in certain examples. In some examples, a surface comprises wells. In some examples, a support structure can include one or more layers. Example support structures can include a chip, a film, a multi-well plate, and a flow-cell.

As used herein, “bead” can refer to a small body made of a solid material. The material of the bead may be rigid or semi-rigid. The body can have a shape characterized, for example, as a sphere, oval, microsphere, or other recognized particle shape whether having regular or irregular dimensions. In some examples, a bead or a plurality of beads can comprise a surface. Example materials that are useful for beads include glass such as modified or functionalized glass; plastic such as acrylic, polystyrene or a copolymer of styrene and another material, polypropylene, polyethylene, polybutylene, polyurethane or TEFLON; polysaccharides or cross-linked polysaccharides such as agarose or Sepharose; nylon; nitrocellulose; resin; silica or silica-based materials including silicon and modified silicon; carbon-fiber; metal; inorganic glass; or a variety of other polymers. Example beads include controlled pore glass beads, paramagnetic beads, thoria sol, Sepharose beads, nanocrystals and others known in the art. Beads can be made of biological or non-biological materials. Magnetic beads are particularly useful due to the ease of manipulation of magnetic beads using magnets at various processes of the methods described herein. Beads used in certain examples can have a diameter, width or length from about 5.0 nm to about 100 μm, e.g., from about 10 nm to about 100 μm, e.g., from about 50 nm to about 50 μm, e.g., from about 100 nm to about 500 nm. In some examples, beads used in certain examples can have a diameter, width or length less than about 100 μm, 50 μm, 10 μm, 5 μm, 1 μm, 0.5 μm, 100 nm, 50 nm, 10 nm, 5 nm, 1 nm, 0.5 nm, 100 μm, or any diameter, width or length within a range of any two of the foregoing diameters, widths or lengths. Bead size can be selected to have reduced size, and hence get more features per unit area, whilst maintaining sufficient signal (template copies per feature) in order to analyze the features.

In some examples, polynucleotides, such as capture probes or codes can be coupled to beads. In some examples, the beads can be distributed into wells on the surface of a substrate, such as a flow cell. Example bead arrays that can be used in certain examples include randomly ordered BEADARRAY technology (Illumina Inc., San Diego Calif.). Such bead arrays are disclosed in Michael et al., Anal Chem 70, 1242-8 (1998); Walt, Science 287, 451-2 (2000); Fan et al., Cold Spring Harb Symp Quant Biol 68:69-78 (2003); Gunderson et al., Nat Genet 37:549-54 (2005); Bibikova et al. Am J Pathol 165:1799-807 (2004); Fan et al., Genome Res 14:878-85 (2004); Kuhn et al., Genome Res 14:2347-56 (2004); Yeakley et al., Nat Biotechnol 20:353-8 (2002); and Bibikova et al., Genome Res 16:383-93 (2006), each of which is incorporated by reference in its entirety.

As used herein, a “polymer” refers to a molecule including a chain of many subunits that are coupled to one another and that may be referred to as monomers. The subunits may repeat, or may differ from one another. Polymers can be biological or synthetic polymers. Example biological polymers that suitably can be included within a bridge or a label include polynucleotides, polypeptides, polysaccharides, polynucleotide analogs, and polypeptide analogs. Example polynucleotides and polynucleotide analogs suitable for use in a bridge or a label include DNA, enantiomeric DNA, RNA, PNA (peptide-nucleic acid), morpholinos, and LNA (locked nucleic acid). Polymers may include spacer phosphoramidites, which may be coupled to polynucleotides but which lack nucleobases, such as commercially available from Glen Research (Sterling, Va.). Example synthetic polypeptides can include charged or neutral amino acids as well as hydrophilic and hydrophobic residues. Example synthetic polymers that suitably can be included within a bridge or label include PEG (polyethylene glycol), PPG (polypropylene glycol), PVA (polyvinyl alcohol), PE (polyethylene), LDPE (low density polyethylene), HDPE (high density polyethylene), polypropylene, PVC (polyvinyl chloride), PS (polystyrene), NYLON (aliphatic polyamides), TEFLON® (tetrafluoroethylene), thermoplastic polyurethanes, polyaldehydes, polyolefins, poly(ethylene oxides), poly(ω-alkenoic acid esters), poly(alkyl methacrylates), and other polymeric chemical and biological linkers such as described in Hermanson, Bioconjugate Techniques, third edition, Academic Press, London (2013). Synthetic polymers may be conductive, semiconductive, or insulating.

As used herein, DNA with “tertiary structure” is intended to mean DNA that is folded into a three-dimensional tertiary structure having internal cross-linking holding the folds in place. In comparison, DNA that has a primary structure (e.g., a particular sequence of monomers linked together) and a secondary structure (e.g., local structure) but no internal cross-linking holding folds into place would not be considered to have a tertiary structure as the term is used

### Bead-Based System and Methods for Optically Detecting Multiple Analytes

Provided herein are a bead-based “universal” system and methods for detection of multiple analytes, which also may be referred to as providing multiomic detection. Multiple, different analytes (e.g., any suitable combination of any nucleotide analytes and non-nucleotide analytes) may be detected by capturing those analytes using a plurality of different sensing probes that are specific to those analytes, coupling fluorophores to those sensing probes, and then coupling those sensing probes (and the fluorophores coupled thereto) to different, respective beads that are all configured similarly to one another while being specific for the respective sensing probes. For example, each of the sensing probes can include a capture probe that is specific to bind one of the analytes, and a code (such as an oligonucleotide sequence) that is specific to one of the beads. Additionally, each of the beads can include a code (such as an oligonucleotide sequence) that is specific to one of the sensing probes. As such, the sensing probes which had captured analytes, and the fluorophores coupled thereto, become bound to a specified bead that may be decoded. As such, the beads themselves need not be specifically functionalized to bind analytes or fluorophores, but rather may be configured to couple to sensing probes (e.g., may include oligonucleotide sequences that are complementary to oligonucleotide sequences of the sensing probes).

Indeed, the present design provides substantial flexibility in how analyte enrichment may be performed because analyte capture is independent of analyte identification and quantification. The present design easily may be extended to detection of any type of analyte, including any suitable combination of nucleotide analytes and non-nucleotide analytes. Examples of nucleotide analytes include copy number variation, gene expression, RNA splice variants, and methylation, which may be detected using nucleotide-based sensing probes. Examples of non-nucleotide analytes include proteins and metabolites, which may be detected using non-nucleotide based sensing probes (such as antibodies) or with nucleotide-based sensing probes (such as aptamers). On-bead fluorescence detection and decode are performed in the same manner for both nucleotide analytes and non-nucleotide analytes, allowing for a common read-out across different types of analytes on a single system. In addition to supporting such a common read-out, the present system provides a flexible content design that is completely customizable.

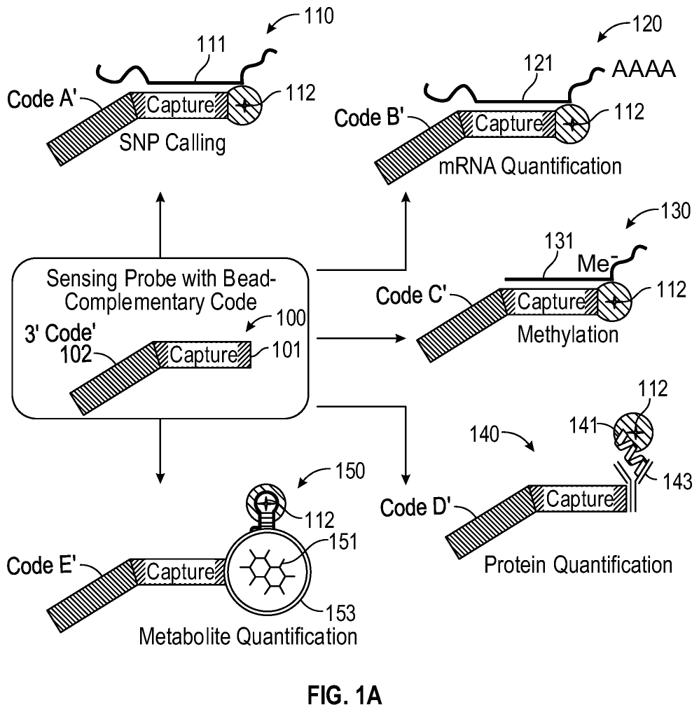

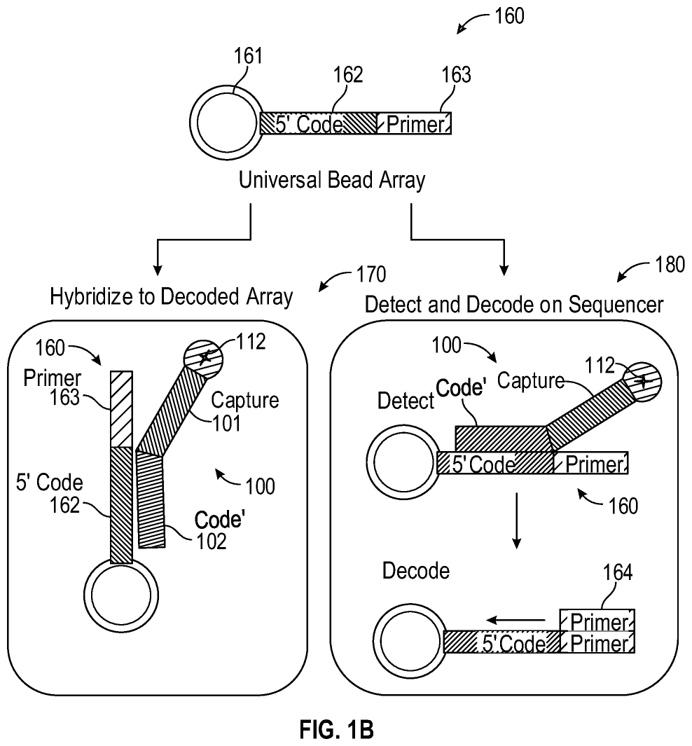

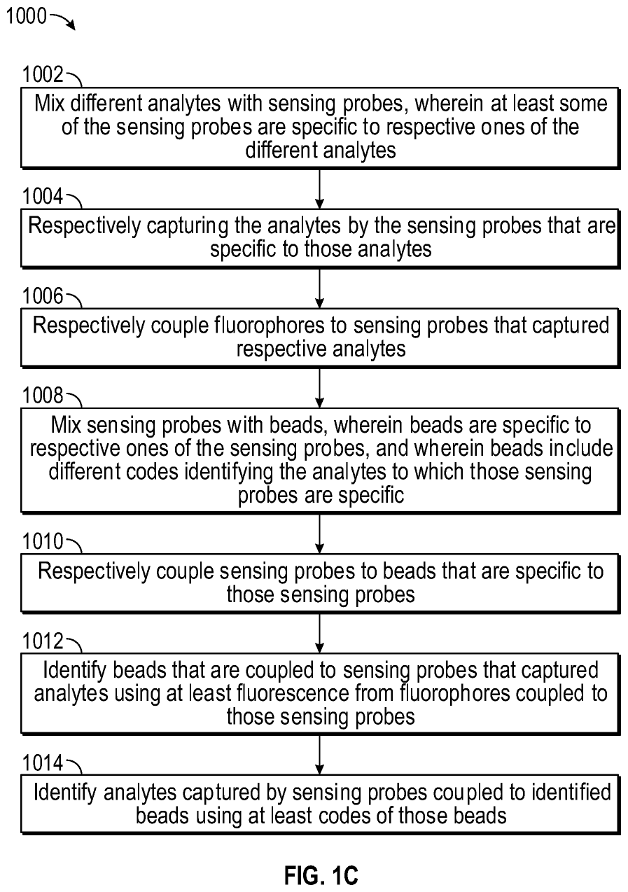

FIGS. 1A-1B schematically illustrate example components of a bead-based system for detecting multiple analytes. The different analytes may include any suitable number and mixture of nucleotide analytes (e.g., zero, one, or a plurality of nucleotide analytes), and any suitable number of non-nucleotide analytes (e.g., zero, one, or a plurality of non-nucleotide analytes). The different analytes may be mixed in a common solution with one another, and may be derived from any suitable source or combination of sources, such as blood, tissue, saliva, urine, or the like.

As illustrated in FIG. 1A, the present system includes different sensing probes 100 that are specific to, and can capture, respective ones of the different analytes. That is, each different sensing probe selectively captures one particular type of analyte in the solution with which such sensing probes are mixed. In some examples, as many different sensing probes may be provided in the solution as the number of different types of analytes it is desired to detect in that solution. For example, if it is desired to detect 10,000 different types of analytes, then 10,000 different sensing probes that are respectively specific to those analytes may be provided. It will be appreciated that any suitable number of different sensing probes may be provided, e.g., more than 100, more than 1,000, more than 10,000, more than 100,000, or more than 1,000,000. It will also be appreciated that any given solution may not necessarily include all possible analytes that it may be desired to detect. As such, some sensing probes may not necessarily have an analyte to capture in a given solution. However, at least some of the sensing probes can capture the analytes to which those sensing probes are specific.

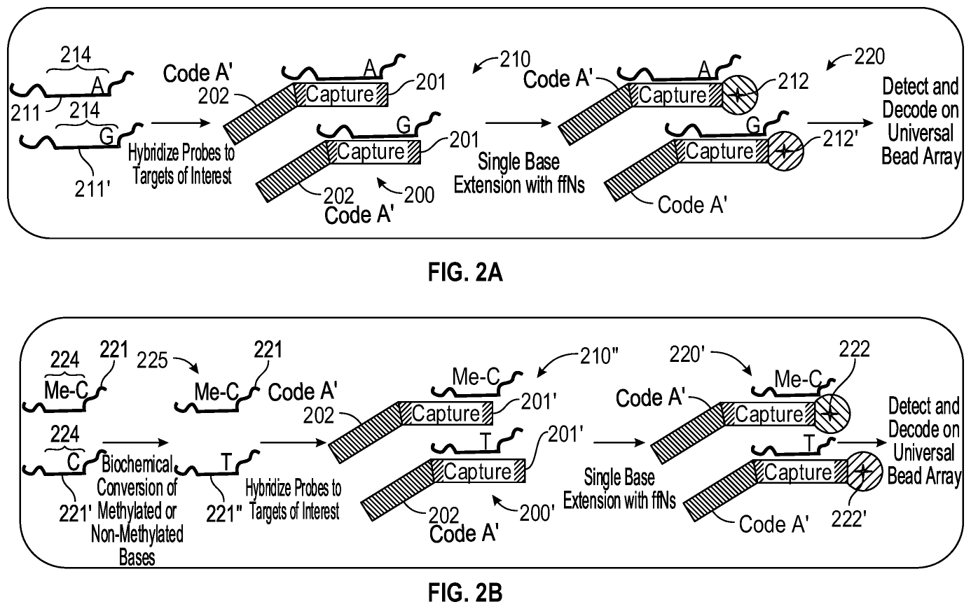

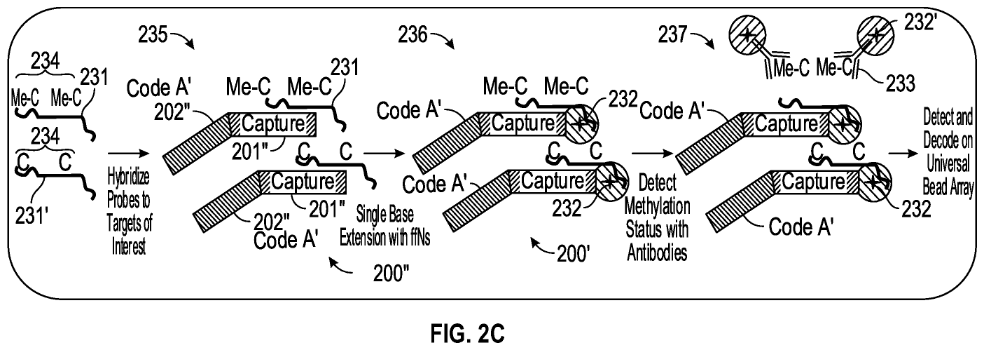

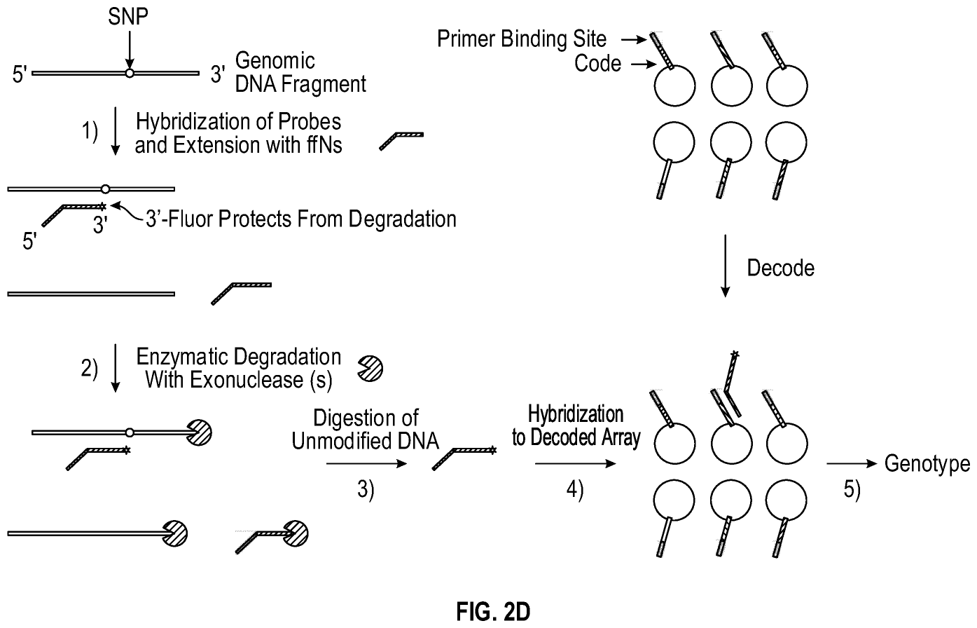

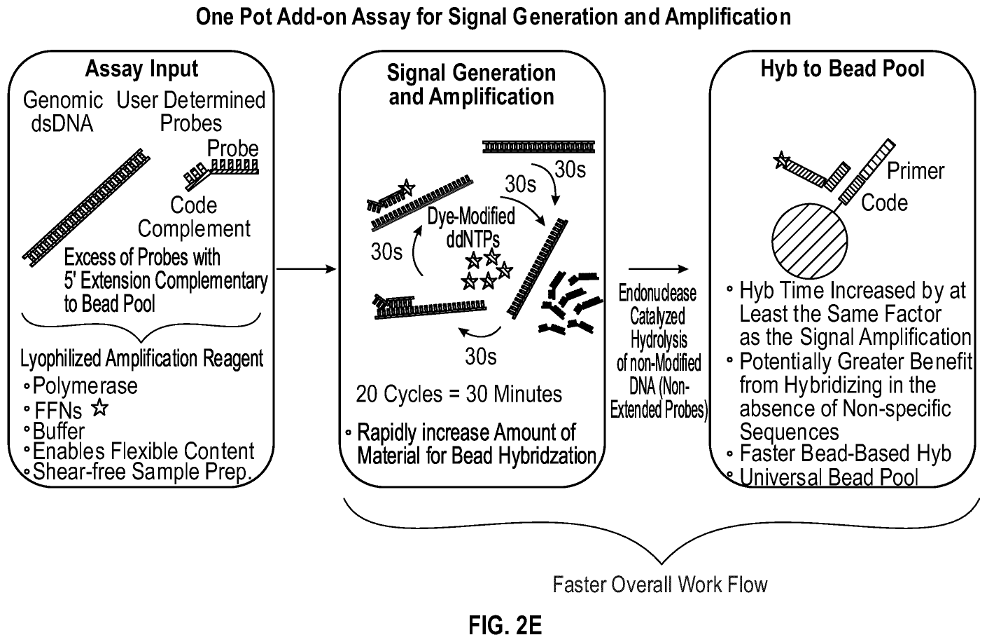

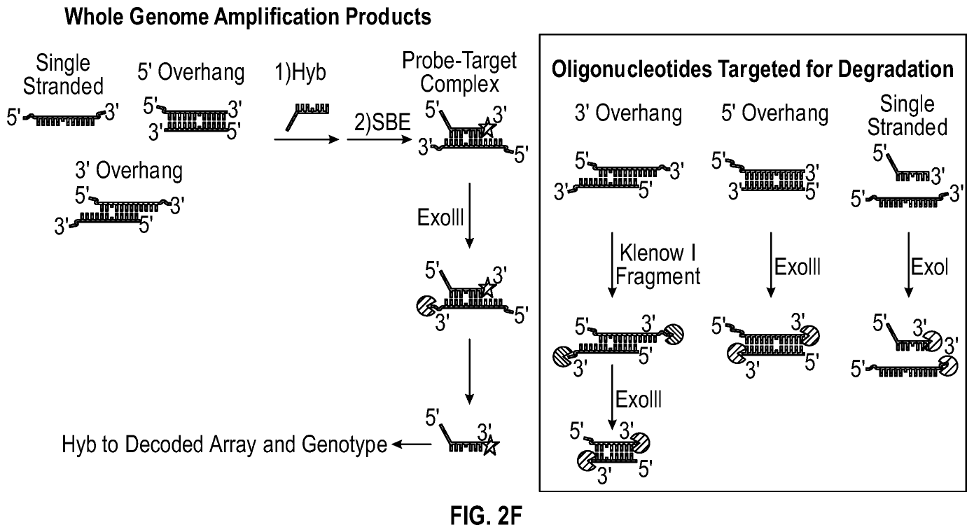

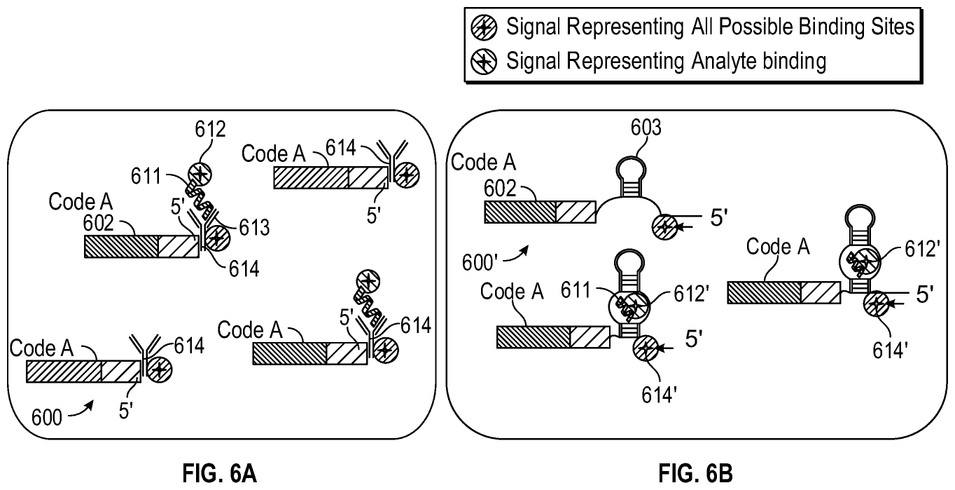

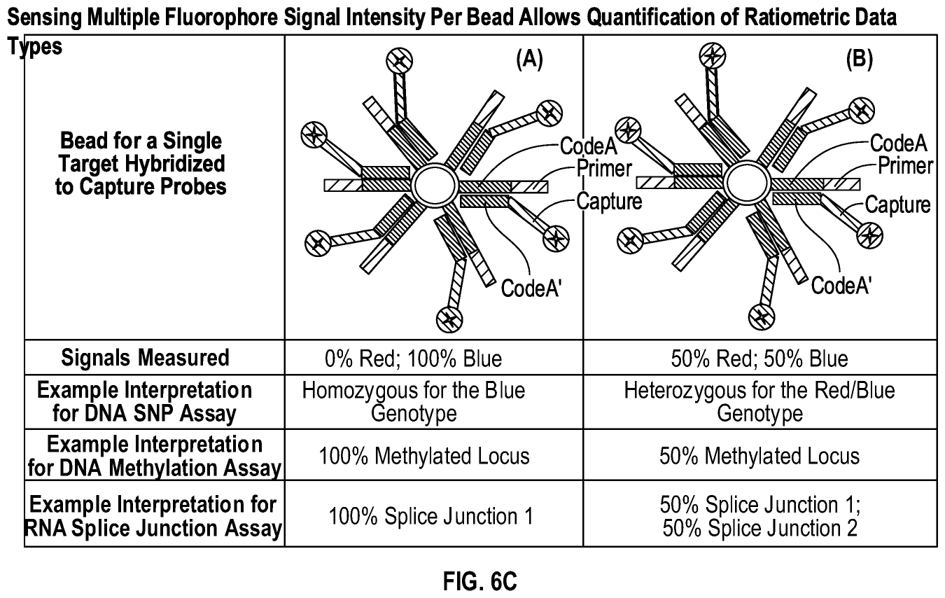

In examples such as illustrated in FIG. 1A, different sensing probes 100 (sensing probe with bead-complementary code) include different capture probes 101 and different codes 102 than one another. Each capture probe 101 may be specific to capture a particular analyte. Some of the analytes may be nucleotide analytes, and some of the analytes may be non-nucleotide analytes. In example 110 in FIG. 1A (SNP calling), one of the capture probes 101 is specific to a first nucleotide analyte, such as a specific DNA sequence 111 for which it is desired to detect a SNP. In example 120 in FIG. 1A (mRNA quantification), one of the capture probes 101 is specific to a second nucleotide analyte, such as a specific mRNA sequence for which it optionally may be desired to detect that sequence's quantity. In example 130 in FIG. 1A (methylation), one of the capture probes 101 is specific to a third nucleotide analyte, such as a specific DNA sequence for which it is desired to detect methylation of a particular nucleotide. In example 140 in FIG. 1A (protein quantification), one of the capture probes 101 is specific to a first non-nucleotide analyte, such as a protein for which it optionally may be desired to detect that protein's quantity. In example 150 in FIG. 1A (metabolite quantification), one of the capture probes 101 is specific to a second non-nucleotide analyte, such as a metabolite for which it optionally may be desired to detect that metabolite's quantity. One or more of the capture probes may include an oligonucleotide. The oligonucleotide may hybridize with a nucleotide analyte, or may provide an aptamer that may capture a non-nucleotide analyte. Additionally, or alternatively, one or more of the capture probes may include a non-oligonucleotide moiety, such as an antibody, to capture a non-nucleotide analyte. Fluorophores may be coupled to sensing probes 110 that captured respective ones of the different analytes. For example, in each of examples 110, 120, 130, 140, and 150, fluorophore 112 is coupled to the sensing probes that captured the respective analytes. Further details of example manners in which different sensing probes may respectively capture different analytes, and may be coupled to fluorophores, are provided below with reference to FIGS. 2A-2F, 3A-3B, 4A-4B, and 5A-5C, and further details of the manner in which the quantities of analytes may be detected are provided below with reference to FIGS. 6A-6B.

Referring now to FIG. 1B, the present system also includes different beads 160 (together providing a universal bead array) that are specific to, and can couple to, respective ones of the different sensing probes. That is, each different bead selectively couples to one particular type of sensing probe in the common solution. In some examples, as many different beads 160 in the universal bead array may be provided in the present system as the number of different types of analytes it is desired to detect. For example, if it is desired to detect 10,000 different types of analytes, then 10,000 different beads that are respectively specific to sensing probes that, in turn, are specific to and can capture those analytes may be provided. It will be appreciated that any suitable number of different beads may be provided, e.g., more than 100, more than 1,000, more than 10,000, more than 100,000, or more than 1,000,000. It will also be appreciated that a particular solution may not necessarily include all possible analytes that it may be desired to detect, but may include a complete set of sensing probes. As such, some beads may be coupled to sensing probes that may not necessarily have captured an analyte. However, at least some of the beads can be coupled to sensing probes that have captured the analytes to which those sensing probes are specific.

In some examples, each bead 160 in the universal bead array has the same components as each other bead, regardless of the particular analyte that the sensing probes can capture. For example, each bead 160 illustrated in FIG. 1B includes substrate 161 and an oligonucleotide including code 162 and primer 163. Codes 162 of different beads 160 have different oligonucleotide sequences than one another that can selectively couple to respective ones of the different codes 102 of sensing probes 100. Such codes 162 respectively identify the analytes to which those sensing probes are specific, and thus can be used to identify which analytes are captured from the common solution, and optionally also used to quantify those analytes. For example, in a manner such as indicated at process 170 illustrated in FIG. 1B (hybridize to decoded array), each of the beads 160 includes oligonucleotide 162 having a sequence specific to one of the sensing probes 100, and each of the sensing probes 100 includes oligonucleotide 102 having a sequence that is complementary to oligonucleotide 162. Note that capture probe 101 of sensing probe 100 may not necessarily hybridize to primer 162 of bead 160, and instead the end of capture probe 101 may extend into the solution.

As noted above, fluorophores 112 are coupled only to sensing probes 100 that captured an analyte to which those sensing probes are specific. As such, fluorophores 112 become coupled to bead 160 via those sensing probes 110, to which those beads 160 are specific. The beads 160 can be coupled to a surface, e.g., immobilized to a surface within a flow cell. In some examples, such coupling of beads 160 to a surface may be performed before the sensing probes 100 are coupled to the beads; for example, a solution including sensing probes 100 may be flowed over the beads coupled to the surface, and the beads may capture from the solution the sensing probes to which those beads are specific. In other examples, such coupling of beads 160 to a surface may be performed after the sensing probes 100 are coupled to the beads; for example, a solution including sensing probes 100 may be mixed with a solution including beads 160 resulting in respective couplings between beads 160 and the sensing probes to which those beads are specific, and the beads subsequently may be coupled to a surface, for example using bioorthogonal conjugation chemistries such as copper(I)-catalyzed click reaction (between azide and alkyne), strain-promoted azide-alkyne cycloaddition (between azide and DBCO (dibenzocyclooctyne), hybridization of an oligonucleotide to a complementary oligonucleotide, biotin-streptavidin, NTA-His-Tag, or Spytag-Spycatcher, charge-based immobilization such as amino-silane or poly-lysine, or non-specific such as with a polymer-coated surface.

As illustrated at process 180 illustrated in FIG. 1B (detect and decode on sequencer), the beads then can be detected via fluorescence from fluorophores 112, e.g., using a suitable imaging camera and detection circuit. Using at least the detected fluorescence, beads 160 can be identified that are coupled to sensing probes 100 that had captured an analyte (detect operation); in comparison, beads 160 that are coupled to sensing probes 100 that had not captured an analyte may not be coupled to a fluorophore, and thus not detected via fluorescence. The sensing probes 110 and fluorophores 112 then may be removed, e.g., by dehybridization, a primer 164 coupled to primer region 163 of bead 160, and code 162 then decoded using sequencing by synthesis or other suitable method. For example, fluorescently labeled nucleotides can be added to primer 164 in a sequence that is complementary to the sequence of code 162. The identity of the analyte may be determined using at least the sequence of code 162. For example, the detection circuit may include memory storing different codes 162 and the analytes corresponding to those codes, and may be configured to compare the sequence of code 162 to the stored codes and to determine the analyte corresponding to the code of bead 160 (decode operation).

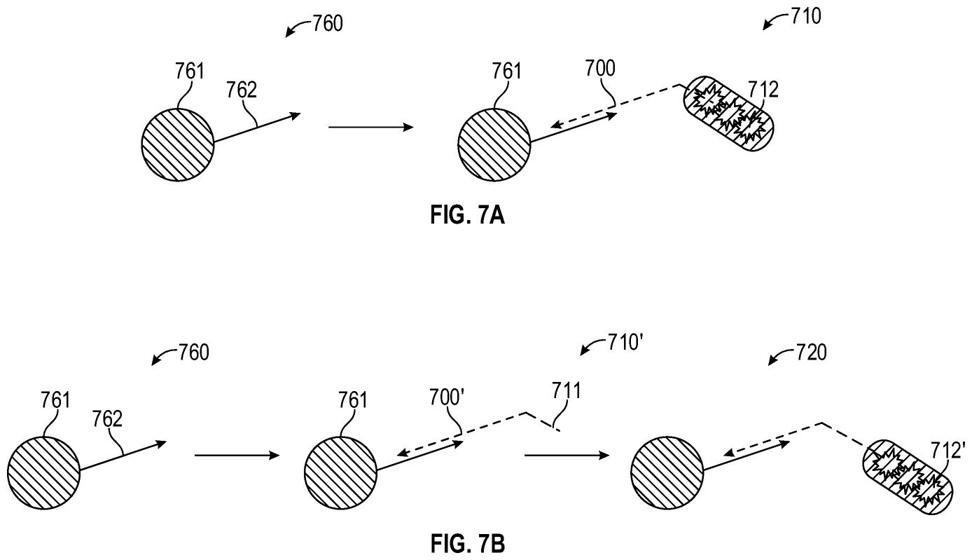

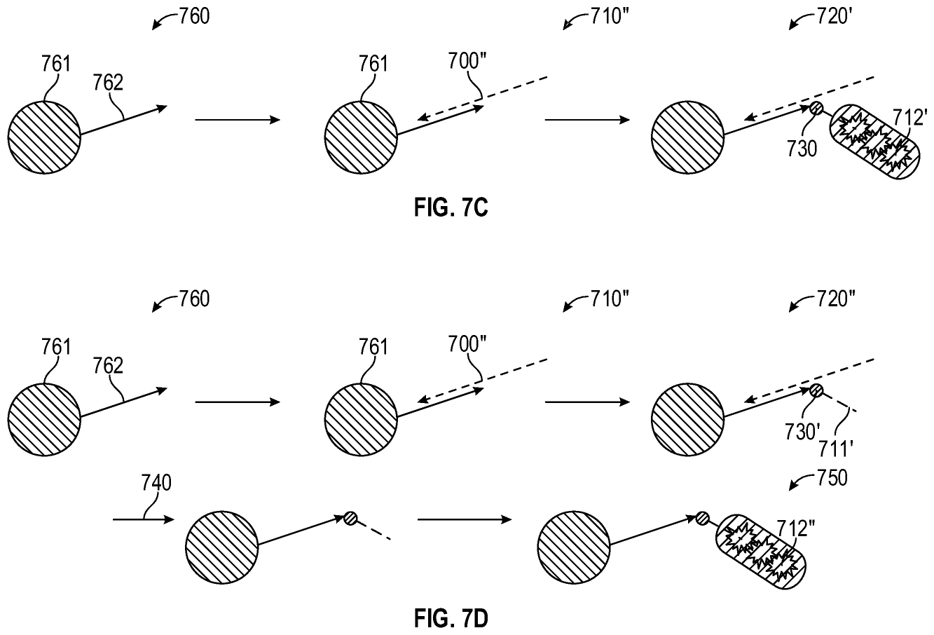

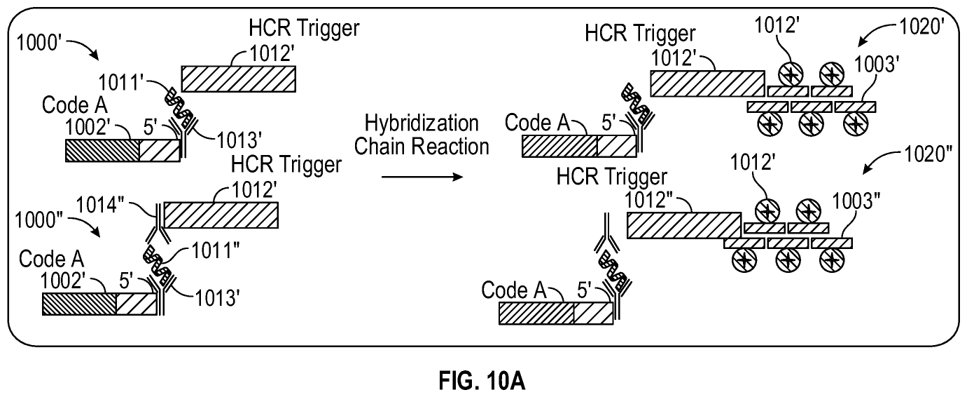

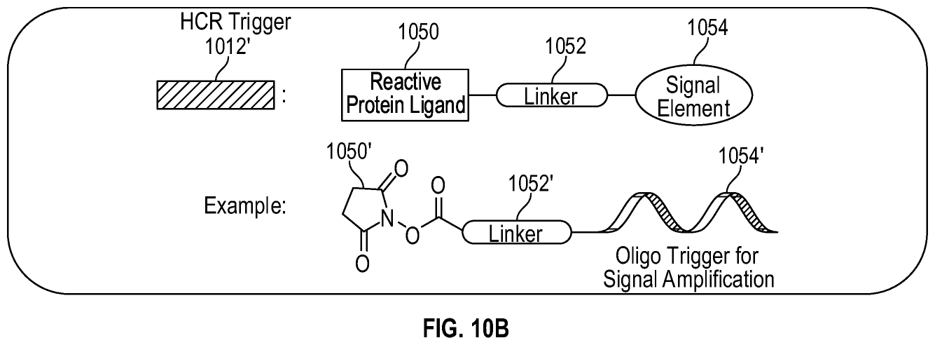

Note that fluorophores 112 may be coupled to respective sensing probes 100 at any suitable time during process flows such as illustrated in FIGS. 1A-1B. For example, fluorophores 112 may be coupled to the sensing probes after the analytes are captured by the sensing probes, e.g., may be coupled to capture probe 101 using at least the sequence of nucleotide analyte 112, 121, 131. Or, for, example, fluorophores may be coupled to the sensing probes before the sensing probes are coupled to the beads, e.g., may be coupled to protein 141 prior to capture of that protein by antibody 143, or may be coupled to metabolite 151 prior to coupling of the sensing probe to the bead. Or, for example, fluorophores 112 may be coupled to the sensing probes after the sensing probes are coupled to the beads, e.g., in a manner such as described with reference to FIGS. 7A-7B. In some examples, multiple fluorophores are coupled to the analytes, e.g., using a hybridization chain reaction (HCR) in a manner such as described with reference to FIG. 10A. Other examples of coupling multiple fluorophores to analytes are provided elsewhere herein.

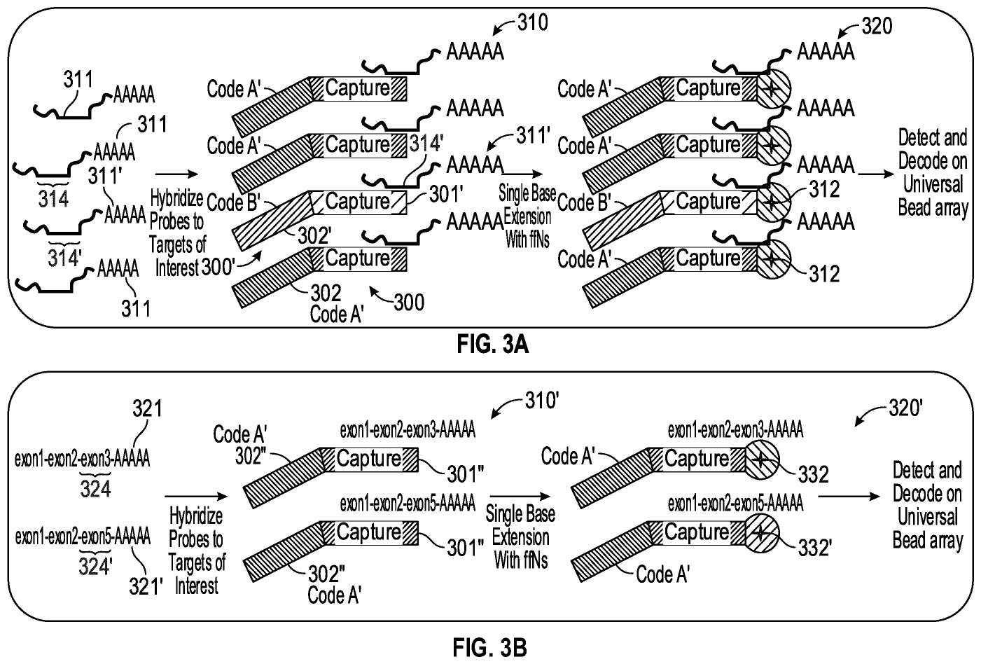

FIG. 1C schematically illustrates an example process flow 1000 for detecting multiple analytes in a bead-based system. Process flow 1000 illustrated in FIG. 1C includes mixing different analytes with sensing probes, wherein at least some of the sensing probes are specific to respective ones of the analytes (process 1002). Examples of sensing probes that are specific to respective analytes are provided elsewhere herein, e.g., with reference to FIGS. 3A-3B, 4A-4B, and 5A-5C. The sensing probes may be provided in excess relative to the respective analytes, so as to increase the likelihood that each given sensing probe captures the analyte to which that probe is specific. For example, the sensing probes may be provided in an excess of greater than 10 times, greater than 100 times, greater than 1,000 times, or greater than 10,000 times in excess of the analytes to which those probes are specific. Illustratively, a given analyte may have a concentration of 1-10 pM, and the sensing probe specific to that analyte may have a concentration of greater than 10 nM, e.g., 10-100 nM. Process flow 1000 illustrated in FIG. 1C includes respectively capturing the analytes by the sensing probes that are specific to those analytes (process 1004). Some of the sensing probes in the mixture may be specific for analytes that are not necessarily present in the mixture, and thus will not be coupled to such analytes. Process flow 1000 includes respectively coupling fluorophores to sensing probes that captured respective analytes (process 1006). Example manners in which fluorophores may be coupled to sensing probes are described elsewhere herein.

Process flow 1000 illustrated in FIG. 1C includes mixing the sensing probes with beads, wherein the beads are specific to respective ones of the sensing probes, and wherein the beads include different codes identifying the analytes to which those sensing probes are specific (process 1008). Such mixing may occur by combining sensing probes in solution with beads in solution. Alternatively, such mixing may occur by flowing a solution that includes sensing probes over beads that are coupled to a surface. Process flow 1000 includes respectively coupling sensing probes to beads that are specific to those sensing probes (process 1010). For example, each given bead may include a plurality of codes which are the same as one another and that respectively are selective couple to the code of a given sensing probe. Accordingly, any sensing probes in the solution may become selectively coupled to that bead. Process flow 1000 includes identifying the beads that are coupled to the sensing probes that captured analytes using at least fluorescence from the fluorophores coupled to those sensing probes (process 1012). For example, the beads may be coupled to a surface (e.g., before or after being coupled to respective sensing probes) and regions of fluorescence on that surface may be imaged. Process flow 1000 includes identifying the analytes that are captured by the sensing probes coupled to the identified beads using at least the codes of those beads (process 1014). For example, the codes of the beads may be decoded using sequencing-by-synthesis, and the decoded codes used to determine which analyte was specific to the sensing probe to which the bead was specific.

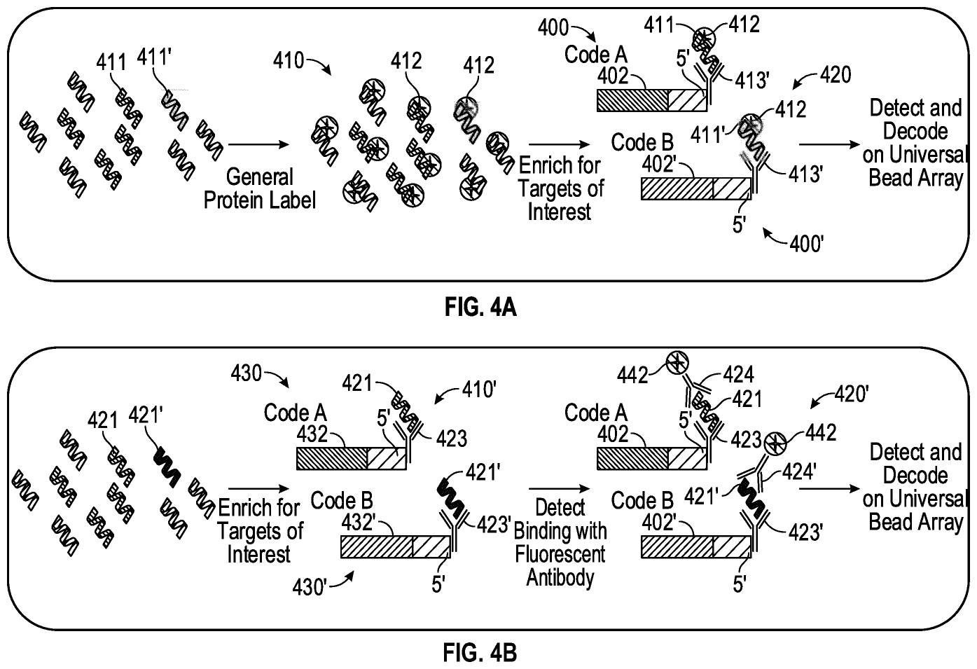

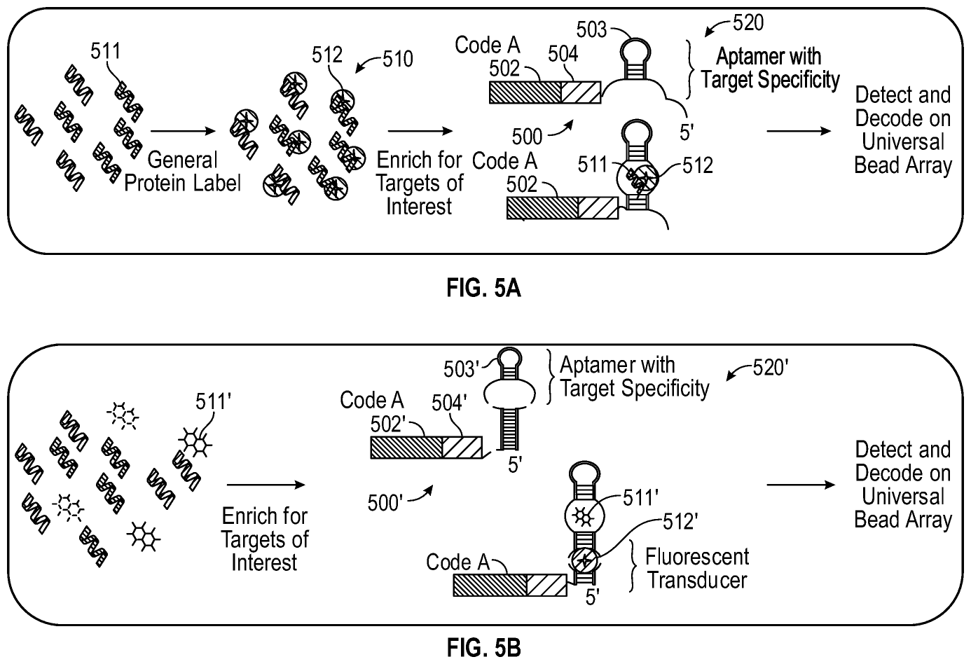

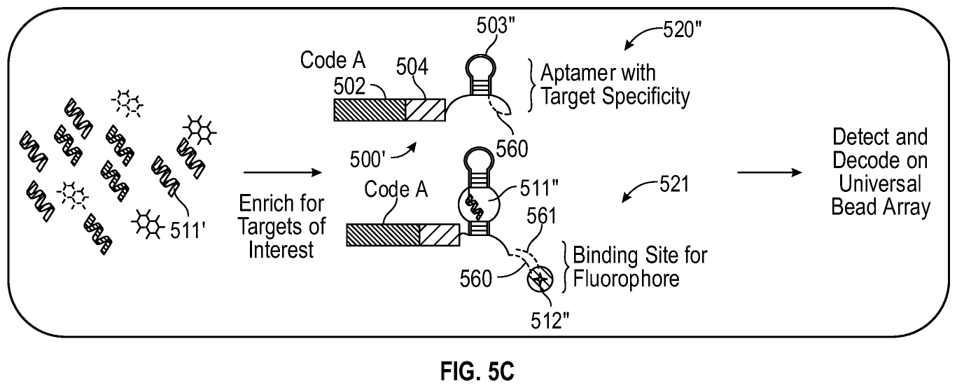

Some non-limiting examples of analytes and sensing probes for specifically capturing such analytes, now will be described. It should be appreciated that the present sensing probes suitably may be modified to respectively capture any suitable analyte with specificity. Example nucleotide analytes are described with reference to FIGS. 2A-2F and 3A-3B, and example non-nucleotide analytes are described with reference to FIGS. 4A-4B and 5A-5C. Any suitable combination of such analytes may be detected using the present systems and methods. For example, a solution that is mixed with the sensing probes may include one or more non-nucleotide analytes, or may include one or more nucleotide analytes. For example, the solution may include a mixture of nucleotide analytes and non-nucleotide analytes. In some examples, the different analytes are mixed together in a solution, and portions of that solution are mixed with respective types of sensing probes that target respective types of analytes. For example, a first portion of the solution may be mixed with sensing probes that are specific to one or more types of nucleotide analytes, and a second portion of the solution may be mixed with sensing probes that target one or more other types of nucleotide analytes. Or, for example, a first portion of the solution may be mixed with sensing probes that are specific to one or more types of nucleotide analytes, and a second portion of the solution may be mixed with sensing probes that target one or more types of non-nucleotide analytes. Or, for example, a first portion of the solution may be mixed with sensing probes that are specific to one or more types of non-nucleotide analytes, and a second portion of the solution may be mixed with sensing probes that target one or more other types of non-nucleotide analytes.

In some examples, a sensing probe can include an oligonucleotide sequence specific to hybridize to a nucleotide analyte, such as a DNA analyte or RNA analyte. For example, FIGS. 2A-2C schematically illustrate example hybridization-based process flows for detecting DNA analytes in a bead-based system. In the example illustrated in FIG. 2A, DNA analytes 211, 211′ include DNA sequences that differ from one another by a SNP that it is desired to detect. For example, DNA analyte 211 includes sequence 214 with A at a given location, while DNA analyte 211′ the same sequence but with G instead of A at the given location, and it is desired to detect the respective presence of the A and G in that sequence 214. As illustrated in FIG. 2A, sensing probes 200 are hybridized to these targets of interest at process 210 (hybridize probes to targets of interest). More specifically, one copy of sensing probe 200 may be hybridized to DNA analyte 211, and another copy of sensing probe 200 may be hybridized to DNA analyte 211′. In this example, each sensing probe 200 includes capture probe 201 including a sequence that is complementary to sequence 214 of DNA analytes 211, 211′ but that terminates at the nucleotide immediately preceding the location with the SNP (e.g., A or G) that it is desired to detect. Each sensing probe 200 also can include the same code 202 as one another which can be coupled to a specific bead in a manner such as described with reference to FIGS. 1A-1C. In some examples, the DNA analytes (e.g., the SNP that it is desired to detect) are detected via differences in fluorescence that are caused by differences between the analytes. Illustratively, at process 220, the respective capture probes 201 of the sensing probes 200 are each extended by a single base with fully functional nucleotides (ffNs) that are fluorescently labeled (single base extension with ffNs). Because the sequences of DNA analytes 211, 211′ differ from one another by the SNP (e.g., A or G), addition of differently fluorescently labeled ffNs to the location with that SNP result in different optical signals that may be distinguished from one another. The sensing probes can be coupled to one or more beads, e.g., to respective beads, optically detected, and the corresponding beads decoded in a manner such as described with reference to FIGS. 1A-1C (detect and decode on universal bead array).

The present systems and methods also may be used to detect and quantify DNA methylation in any suitable manner. For example, biochemical conversion of methylated or non-methylated nucleotides to a different base (for example, with bisulfite treatment) can be performed before capturing the analyte with a sensing probe. After capture, a single base extension is performed with ffNs at the site of potential methylation; the methylation status can be determined by the ffN-fluorophore that was incorporated. Such a workflow may provide for single-based resolution of DNA methylation.

For example, as illustrated in FIG. 2B, DNA analytes 221, 221′ include DNA sequences that differ from one another by a nucleotide methylation that it is desired to detect. In this non-limiting example, DNA analyte 221 includes sequence 224 with methylated-C(Me-C) at a given location, while DNA analyte 221′ includes the same sequence but with non-methylated C at the given location, and it is desired to detect the respective presence of the methylation in that sequence 224. At process 225, either the methylated or non-methylated nucleotide is selectively converted to a different base, for example with bisulfite treatment (biochemical conversion of methylated or non-methylated bases). Here, the non-methylated C is selectively converted to T, while Me-C is unchanged by the treatment due to the methylation. As illustrated in FIG. 2B, sensing probes 200′ are hybridized to these targets of interest at process 210″ (hybridize probes to targets of interest). More specifically, one copy of sensing probe 200′ may be hybridized to DNA analyte 221, and another copy of sensing probe 200′ may be hybridized to DNA analyte 221′. In this example, each sensing probe 200′ includes capture probe 201′ including a sequence that is complementary to sequence 224 of DNA analytes 221, 221′ but that terminates at the nucleotide immediately preceding the location with the methylation that it is desired to detect. Each sensing probe 200′ also can include the same code 202′ as one another which can be coupled to a specific bead in a manner such as described with reference to FIGS. 1A-1C. In some examples, the DNA analytes (e.g., the methylation that it is desired to detect) are detected via differences in fluorescence that are caused by differences between the analytes. Illustratively, at process 220′, the respective capture probes 201′ of the sensing probes 200′ are each extended by a single base with ffNs 222, 222′ that are fluorescently labeled (single base extension with ffNs). Because the sequences 224 of DNA analytes 221, 221′ differ from one another as a result of the methylation and conversion (Me-C or T), addition of differently fluorescently labeled ffNs 222, 222′ to the location with that methylation result in different optical signals that may be distinguished from one another. The sensing probes can be coupled to one or more beads, e.g., to respective beads, optically detected, and the corresponding beads decoded in a manner such as described with reference to FIGS. 1A-1C (detect and decode on universal bead array).

In another example of methylation detection, target DNA can be hybridized to the capture probe without prior processing, and then a single base extension is performed with ffNs. After the extension, fluorophore-conjugated antibodies against the methylated target nucleotide are added, which bind the methylated bases. Total target capture may be quantified by the fluorescence intensity of the ffN, and the extent of methylation may be measured by the fluorescence intensity of the antibody-fluorophore. This approach may not necessarily allow for single base resolution, as antibodies may bind all methylated nucleotides in proximity to the capture site. However, the approach may be performed without upfront biochemical processing of the sample DNA, and for assessing regions with many methylation events, multiple antibody binding events may amplify the fluorescent signal.

For example, as illustrated in FIG. 2C, DNA analytes 231, 231′ include DNA sequences that differ from one another by one or more nucleotide methylations that it is desired to detect. In this non-limiting example, DNA analyte 231 includes sequence 234 with methylated-C(Me-C) at one or more given locations, while DNA analyte 231′ includes the same sequence but with non-methylated Cs at the given locations, and it is desired to detect the respective presence of the methylation(s) in that sequence 234. As illustrated in FIG. 2C, sensing probes 200″ are hybridized to these targets of interest at process 235 (hybridize probes to targets of interest). More specifically, one copy of sensing probe 200″ may be hybridized to DNA analyte 231, and another copy of sensing probe 200″ may be hybridized to DNA analyte 231′. In this example, each sensing probe 200″ includes capture probe 201″ including a sequence that is complementary to sequence 234 of DNA analytes 231, 231′ but that terminates at the nucleotide immediately preceding the location with one of the methylations that it is desired to detect. Each sensing probe 200″ also can include the same code 202″ as one another which can be coupled to a specific bead in a manner such as described with reference to FIGS. 1A-1C. In some examples, the DNA analytes (e.g., the methylation that it is desired to detect) are detected via differences in fluorescence that are caused by differences between the analytes. Illustratively, at process 236, the respective capture probes 201″ of the sensing probes 200″ are each extended by a single base with ffNs 232 that are fluorescently labeled (single base extension with ffNs). Because the sequences 234 of DNA analyte 231, 231′ are the same as one another except for the methylation(s) (Me-C), the same fluorescently labeled ffNs 232 will be added to the terminal location with that methylation. In this example, at process 237 fluorescently labeled antibodies 232′ are added to detect the methylation status of sequence 234 (detect methylation status with antibodies). For example, the fluorescently labeled antibodies 232 may selectively bind to Me-C. The sensing probes then can be coupled to one or more beads, e.g., to respective beads, fluorescence from the differently fluorescently labeled sensing probes optically detected, and the corresponding beads decoded in a manner such as described with reference to FIGS. 1A-IC (detect and decode on universal bead array).

Some examples of the methods and systems provided herein relate to the detection of target nucleic acids, which also may be referred to as DNA analytes. In some examples, target nucleic acids are detected by hybridizing a plurality of nucleic acids comprising target nucleic acids to probes (sensing probes) capable of hybridizing to the target nucleic acids; extending the hybridized probes; and detecting the extended probes, thereby detecting the target nucleic acids. In some examples, the hybridization and extension processes are performed in solution. In other examples they are performed in conjunction with a solid support, such as a microfluidics device. In some examples, the extended probes are enriched by removing unextended probes, and optionally the plurality of nucleic acids, from the extended probes. In some examples, the extended probes are detected by hybridizing the extended probes to an array of capture probes immobilized on a surface. In some examples, an array of capture probes is a decoded array wherein each capture probe has a unique signature or bar code and the position of each capture probe is decoded prior to use. In some examples, the array of capture probes comprises a universal array.

Some examples provided herein include methods for increasing the performance of a genotyping assay and enabling the use of universally decoded arrays by using a sample preparation strategy that may employ solution-phase target capture, probe extension, and enrichment, followed by bead-based genotyping. Some such examples address known challenges including the inefficient capture of DNA targets for genotyping; template-independent probe extension and increased background signal due to high local concentrations of probes after immobilization on beads; and the ability to use universal arrays to detect target nucleic acids. In some examples, such challenges are solved by performing target capture and probe extension in solution, followed by enzymatic enrichment of targets-of-interest and introduction to arrays; by performing target capture and first base extension in solution; and by decoupling probe sequences from immobilized oligonucleotides.

Challenges associated with inefficient hybridization in certain methods to detect target nucleic acids include performing target capture on pre-assembled arrays, for example hybridizing target nucleic acids to target-specific probes immobilized on an array. In one example, performing target capture on pre-assembled arrays can place a limit on the probe:target ratio because the number of target-specific probes in this example may be ultimately fixed by the number of beads loaded into the array. Additionally, samples used for genotyping can contain an excess of non-targeted DNA, may be viscous due to high DNA concentrations, and can potentially suffer from re-hybridization of targets to their solution-phase complements. In some examples these challenges are addressed by hybridizing target-specific probes to target nucleic acids in solution. In some examples, in-solution hybridization can enable the use of a large probe:target ratio, which can lead to an increase in hybridization kinetics. In some examples, biochemical enrichment of sequences of interest prior to introduction to the array addresses these challenges by removing oligonucleotides that may negatively affect hybridization.

Challenges associated with inefficient DNA concentrations in samples in certain methods to detect target nucleic acids can limit genotyping performance. Low DNA concentrations may utilize a whole genome amplification process to obtain sufficient concentrations of sample, prior to introduction of DNA samples onto arrays. In some examples provided herein such challenges are addressed by increasing hybridization efficiency by removing non-targeted DNA. In some examples, the amount of extended target-specific probes can be selectively, ultimately increasing the rate of bead-based capture of extended probes.

In certain methods to identify target nucleic acids, biochemistry on immobilized probes can be complicated by surface architecture, for example beads used in certain commercial arrays can contain high local concentrations of probes, which promote inter-oligo interactions and lead to an increase in background signal due to off target incorporation. Optimization of probe surface density can prevent these interactions to some extent but ultimately may involve a tradeoff between optimizing the bead architecture for target capture and preventing non-targeted probe extension. Additionally, the bead presents a surface that nucleotides can bind to, which can lead to an elevated noise level. In some examples provided herein such challenges are addressed by performing probe-target hybridization and extension reactions in solution, such that target concentration gradients and adsorption of reagents to surfaces are minimized.

In certain methods to identify target nucleic acids, commercial array formats may not allow for easily adding custom probes or designing custom genotyping panels because beads are pooled in large batches prior to loading on arrays. In some examples provided herein such challenges are addressed by performing hybridization in solution to allow use of universal arrays with decode sequences that are complementary to a terminal extension of the solution-phase probes.

One example includes performing all biochemical probe manipulations in solution, as well as an additional sample enrichment process, prior to genotyping on arrays. One advantage provided by this example includes the ability to load arrays with beads that are functionalized with decoded oligonucleotides, and not target-specific probes. This allows an end-user to more easily add custom SNPs to methods and compositions for detecting target nucleic acids using arrays.

An example of a method for identifying a target nucleic acids is depicted in FIG. 2D which includes the following processes: (1) Hybridization of target-specific probe (sensing probe) to target nucleic acid (DNA analyte, such as a genomic DNA fragment including a SNP) in solution which can have an increased probe:target ratio relative to hybridization on an array to promote binding; and single base extension of hybridized probes using fluorophore labeled nucleotides that ultimately act as a signal for genotyping (hybridization of probes and extension with ffNs). A genomic DNA fragment includes the target nucleic acids and contains a single nucleotide polymorphism (SNP), the target specific probe hybridizes at a location immediately adjacent to the SNP. The target-specific probe contains or includes a 3′ end capable of hybridizing to the target nucleic acid, and a 5′ end capable of hybridizing to a capture probe. The target-specific probe hybridizes to the target nucleic acid, and is extended with a single modified nucleotide having a 3′ fluorophore which inhibits degradation of the extended probe by 3′-5′ exonucleases (3′-fluor protects from degradation). (2) (enzymatic degradation with exonuclease(s)) and (3) (digestion of unmodified DNA) Enrichment of fluorophore-labeled probes in which 3′-OH specific exonucleases degrade unextended probes and any oligonucleotides that are not intended for capture and genotyping on arrays. Non-extended probes and genomic DNA fragments are degraded by 3′-5′ exonucleases. (4) Hybridization of fluorescent extended probes to arrays via sequence complementary to decode the polynucleotides (hybridization to decoded array). Each target-specific probe contains a sequence at its 5′ terminus that is complementary to a decoded sequence that identifies the position of a particular bead type within an array. These sequences enable hybridization of fluorophore-labeled probes to specific sites on the array for genotyping; the beads include primer binding sites and codes. (5) Genotyping is performed either by direct detection of fluorescent nucleotides or, if necessary or appropriate, after additional signal amplification. Note that although the array may be decoded prior to hybridization of the fluorescent extended probes at (4) in a manner such as shown in FIG. 2D, the target-extended probes instead may be hybridized to a suspension of beads, loaded onto an array, and the beads then decoded.

An example of a method for identifying a target nucleic acids is depicted in FIG. 2E (one pot add-on assay for signal generation and amplification). The left panel of FIG. 2E (assay input) depicts probes (sensing probes, user determined probes including probe and code complement) with a 5′ overhang complementary to a universal bead pool are mixed with a genomic DNA sample (genomic dsDNA) and amplification reagent (excess of probes with 5′ extension complementary to bead pool; lyophilized amplification reagent—polymerase, FFNs, buffer; enables flexible content and shear-free sample prep). The center panel of FIG. 2E (signal generation and amplification) depicts thermal cycling of a mixture to increase the concentration of extended probes, which ultimately enhances bead-based capture of sequences of interest. For example, target-specific probes are hybridized to target nucleic acids, and extended. The extended probes are dehybridized from the target nucleic acids. More target-specific probes are hybridized to target nucleic acids, and extended. The cycles are repeated to amplify the number of extended probes, for example by 20 cycles (for example, at about 30 seconds per process, for a total of about 30 minutes for 20 cycles). Such processes can rapidly increase the amount of material for bead hybridization. After signal generation and amplification, extended probes are enriched by exonuclease-catalyzed degradation of genomic DNA and unextended probes (endonuclease catalyzed hydrolysis of non-modified DNA (non-extended probes). The right panel of FIG. 2E (hyb to bead pool) depicts bead-based capture and genotyping of extended probes. Hybridization time may be increased by at least the same factor as the signal amplification. There potentially a greater benefit from hybridizing in the absence of non-specific sequences. Thus, faster bead-based hybridization is provided, as is a universal bead pool.

Examples of aspects of enriching for extended probes are depicted in FIG. 2F. Whole genome amplification products may include a mixture of single stranded DNA, duplex DNA with 5′ overhang, and duplex DNA with 3′ overhang. The single-stranded DNA in the whole genome amplification products may be hybridized to sensing probes at process 1) followed by single base extension (SBE) with 3′-fluorophore labeled ffNs at process 2) to form probe-target complexes. Enrichment of probes for genotyping is achieved by selective degradation of oligonucleotides that are not 3′-fluorophore labeled (oligonucleotides targeted for degradation). This is enabled by the highly specific nature of restriction exonucleases that each target specific impurities. The following exonucleases are examples of classes which can be utilized to enrich for selected oligonucleotides: (1) Klenow I fragment targets 3′ duplex DNA containing 3′-overhangs; (2) Exonuclease III (ExoIII) targets the 3′ end of duplex DNA; and (3) Exonuclease I (ExoI) degrades single stranded library fragments as well as unreacted primers. Probe-target complexes that have been extended with 3′-fluorophore ffNs may be hybridized to a decoded array (or non-decoded array) and genotype processes performed.

In some examples, it is possible that non-specific incorporation of nucleotides to the 3′ termini of library fragments may occur. In this case, probes may be designed with, for example, phosphorothioate bonds at their 5′-termini. This would allow for selectively degrading library fragments. Some examples include the use of target-specific probes having a 5′ end resistant to enzymatic degradation.

Some examples provided herein include methods for identifying target nucleic acids. Some such examples include (a) hybridizing a plurality of probes to a plurality of nucleic acids comprising the target nucleic acids, wherein each probe comprises a 3′ end capable of hybridizing to a target nucleic acid and a 5′ end capable of hybridizing to a capture probe; (b) extending the hybridized probes with a blocked nucleotide; (c) removing the plurality of nucleic acids and non-extended probes from the extended probes; and (d) hybridizing the extended probes to a plurality of capture probes immobilized on a surface.

In some examples, the capture probes each comprises a 3′ end capable of hybridizing to a target nucleic acid. In some examples, the capture probe is capable of hybridizing to a location on a target nucleic acid immediately proximal to a single nucleotide polymorphism (SNP), or other single nucleotide feature to be examined in the target nucleic acid. In some examples, the 3′ end capable of hybridizing to a target nucleic acid is the most 3′ end of the probe. In some examples, the 3′ end capable of hybridizing to a target nucleic acid is at least 3, 5, 10, 15, 20, 25, 30, 35, 40, 45, 50, 55, 60 consecutive nucleotides in length, or any number of nucleotides between any two of the foregoing numbers. In some examples, the capture probes each comprise a 5′ end capable of hybridizing to a capture probe. In some examples, the 5′ end capable of hybridizing to a target nucleic acid is the most 5′ end of the probe. In some examples, the 5′ end capable of hybridizing to a target nucleic acid is at least 3, 5, 10, 15, 20, 25, 30, 35, 40, 45, 50, 55, 60 consecutive nucleotides in length, or any number of nucleotides between any two of the foregoing numbers. In some examples, the most 5′ end of the probe is resistant to enzymatic degradation. For example, the most 5′ end of the probe can include a phosphorothioate bond.

In some examples, the hybridizing the plurality of probes to a plurality of nucleic acids comprising the target nucleic acids, the extending the hybridized probes with a blocked nucleotide, and the removing the plurality of nucleic acids and non-extended probes from the extended probes, are performed in solution. For example, the probes, the nucleic acids, and the extended probes, are not immobilized on a surface.

In some examples, the amount of extended probe can be increased by performing an amplification process. In some such examples, the plurality of probes is hybridized to the plurality of nucleic acids comprising the target nucleic acids, and the hybridized probes are extended with a blocked nucleotide; and the hybridization and extension repeated. For example, a cycle includes a first hybridization and extension, and then the extended probes are dehybridized from target nucleic acids; non-extended probes are hybridized to the target nucleic acids, the hybridized probes are extended with a blocked nucleotide. In some examples, the cycle is repeated for more than 2, 5, 10, 20, 30 or 50 cycles, or any number between any two of the foregoing numbers.

In some examples, the extension is performed with a polymerase, or a ligase. In some such examples, the extension adds a blocked nucleotide at the most 3′ end of the probe to generate an extended probe. As used herein, a “blocked nucleotide” can include a nucleotide which confers resistance to exonuclease degradation on an extended probe. For example, an extended probe will be resistant to enzymatic degradation by a 3′ to 5′ exonuclease. In some examples, a blocked nucleotide can include a detectable label, such as a fluorophore. In some such examples, the fluorophore which can provide resistance to enzymatic degradation by a 3′ to 5′ exonuclease.

Some examples include removing unextended probes from the extended probes. Some examples also include removing the plurality of nucleic acids and unextended probes from the extended probes. Some such examples include enzymatic degradation of the plurality of nucleic acids and unextended probes. In some examples, the plurality of nucleic acids and the non-extended probes are contacted with a 3′ to 5′ exonuclease. Examples of 3′ to 5′ exonucleases include Exonuclease I, Thermolabile Exonuclease I, Exonuclease T, Exonuclease III, and Klenow I fragment. In some examples, the plurality of nucleic acids and unextended probes are substantially removed from the extended probes, for example, at least 30%, 40%, 50%, 60%, 70%, 80%, 90%, 95%, 99%, 100%, or any percentage between any two foregoing percentages of amounts of the plurality of nucleic acids and unextended probes are at least substantially removed from the extended probes.

In some examples, the probes each comprise a 5′ end resistant to enzymatic degradation, for example, the 5′ end resistant to enzymatic degradation comprises a phosphorothioate bond. In some such examples, the plurality of nucleic acids can be removed from extended probes by contacting the plurality of nucleic acids with a 5′ to 3′ exonuclease. Examples of 5′ to 3′ exonucleases include RecJf, T7 Exonuclease, truncated Exonuclease VIII, Lambda Exonuclease, T5 Exonuclease, Exonuclease VII, Exonuclease V, and Nuclease BAL-31.

Some examples include hybridizing the extended probes to capture probes. IN some examples, the capture probes are immobilized on a surface. In some examples, a bead comprises the surface. In some examples, a plurality of beads comprise the surface. In some examples, a planar surface comprises the surface. In some examples a flow cell comprises the surface. In some examples, a flow cell comprises beads which comprise the surface.

Some examples include amplifying a signal from an extended probe hybridized to a capture probe. In some such examples, a signal is amplified using labelled primary antibodies against the blocked nucleotide, such as the fluorophore. Some examples also include the use of secondary antibodies against the primary antibodies and further labeled.

In some examples, the capture probes are different from each other. For example, different capture probes can be capable of hybridizing to extended probes which have been generated by hybridizing probes to different target nucleic acids. In some examples, the plurality of capture probes comprises a decoded array of capture probes. For example, an array can include a plurality of wells on a surface, each well containing a bead comprising a capture probe. Some examples include decoding the location of the capture probes on a surface. In some examples, the plurality of capture probes each comprises a primer binding site and a decode polynucleotide. In some examples, decoding comprises: hybridizing a sequencing primer to the primer binding site, extending the hybridized primer, and identifying the decode polynucleotide. In some examples, the decode polynucleotide is capable of hybridizing to an extended probe. Some examples include identifying the location of the hybridized extended probes on the surface, such as a surface comprising a decoded array of capture probes, thereby identifying the target nucleic acid.

Some examples provided herein include kits and systems. In some examples, a kit or system for identifying target nucleic acids includes an extension solution comprising: a plurality of nucleic acids comprising the target nucleic acids, a plurality of probes, wherein each probe comprises a 3′ end capable of hybridizing to a target nucleic acid and a 5′ end capable of hybridizing to a capture probe, a plurality of blocked nucleotides, an extension enzyme; a degradation solution comprising a 3′ to 5′ exonuclease; an array of capture probes immobilized on a surface; and a detector to identify (capable of identifying) the location of an extended probe hybridized to a capture probe on the surface. In some examples, a flow cell comprises the array of capture probes immobilized on a surface.

In some examples, a kit or system for identifying target nucleic acids includes a flow cell comprising a surface, an inlet for adding solutions to the surface, and an outlet for removing solutions from the surface, wherein an array of capture probes is immobilized on the surface; an extension solution in contact with the inlet, the extension solution comprising: a plurality of nucleic acids comprising the target nucleic acids, a plurality of probes, wherein each probe comprises a 3′ end capable of hybridizing to a target nucleic acid and a 5′ end capable of hybridizing to a capture probe, a plurality of blocked nucleotides, an extension enzyme; a degradation solution comprising a 3′ to 5′ exonuclease; and a detector to identify (capable of identifying) the location of an extended probe hybridized to a capture probe on the surface.

In some examples, the blocked nucleotide comprises a detectable label. In some examples, the label comprises a fluorophore. In some examples, the extension enzyme comprises a polymerase. In some examples, the extension enzyme comprises a ligase. In some examples, the 3′ to 5′ exonuclease is selected from the group consisting of Exonuclease I, Thermolabile Exonuclease I, Exonuclease T, Exonuclease III, and Klenow I fragment. In some examples, the probes each comprises a 5′ end resistant to enzymatic degradation. In some examples, the 5′ end resistant to enzymatic degradation comprises a phosphorothioate bond. In some examples, the degradation solution further comprises a 5′ to 3′ exonuclease. In some examples, the 5′ to 3′ exonuclease is selected from the group consisting of RecJf, T7 Exonuclease, truncated Exonuclease VIII, Lambda Exonuclease, T5 Exonuclease, Exonuclease VII, Exonuclease V, and Nuclease BAL-31. In some examples, the surface comprises a plurality of beads. In some examples, the capture probes are different from each other. In some examples, the plurality of capture probes comprises a decoded array of capture probes. In some examples, the plurality of capture probes each comprises a primer binding site and a decode polynucleotide. In some examples, the plurality of nucleic acids comprises genomic DNA. In some examples, the target nucleic acids comprise a single nucleotide polymorphism (SNP).

DNA is only one example of a nucleotide analyte that may be detected using the present systems and methods. Similarly as for DNA analytes, a sensing probe can include an oligonucleotide sequence specific to hybridize to an RNA analyte. For example, strategies for the capture and detection of RNA also may be amenable to using cDNA libraries and may be adapted to use RNA directly. In-solution hybridization of RNA (such as cDNA) molecules to a sensing probe including a target identification code followed by single base extension with ffNs, similarly as for DNA workflows, may be used. For example, FIGS. 3A-3B schematically illustrate example hybridization-based process flows for detecting RNA analytes in a bead-based system.

In the example illustrated in FIG. 3A, RNA analytes 311, 311′ include RNA sequences that differ from one another and for which it is desired to detect relative abundances. For example, RNA analyte 311 includes sequence 314, while RNA analyte 311′ includes sequence 314′, and it is desired to detect the abundance of RNA analyte 311 relative to that of RNA analyte 311′. As illustrated in FIG. 3A, sensing probes 300, 300′ respectively are hybridized to these targets of interest at process 310 (hybridize probes to targets of interest). More specifically, one copy of sensing probe 300 may be hybridized to each of RNA analytes 311, and one copy of sensing probe 300′ may be hybridized to RNA analyte 311′. In this example, each sensing probe 300 includes capture probe 301 including a sequence that is complementary to sequence 314 of RNA analyte 311, while each sensing probe 300′ includes capture probe 301′ including a different sequence that is complementary to sequence 314′ of RNA analyte 311′. Each sensing probe 300 also can include the same code 302 as one another which can be coupled to a specific bead in a manner such as described with reference to FIGS. 1A-1C, while each sensing probe 300′ also can include the same code 302′ as one another which can be coupled to a different specific bead in a manner such as described with reference to FIGS. 1A-1C. In some examples, the RNA analytes are detected via differences in the codes of the sensing probes. Illustratively, at process 320 (single base extension with ffNs), the respective capture probes 301 of the sensing probes 300 and capture probes 301′ of the sensing probes 300′ are each extended by a single base with ffNs that are fluorescently labeled. The sensing probes can be coupled to respective beads, optically detected, and the corresponding beads decoded in a manner such as described with reference to FIGS. 1A-1C (detect and decode on universal bead array).

In other examples, the present systems and methods may be used to quantify alternative splicing events and to obtain estimations of transcript isoform abundance. Illustratively, each type of ffN can be coupled to a different fluorophore, and the fluorophore identity can reflect which splicing event took place. Informative measurements of alternative splicing can be obtained by providing a different nucleotide immediately adjacent to the splicing site for each of the possible exons.

In the example illustrated in FIG. 3B, RNA analytes 321, 321′ include RNA sequences that that include different splice isoforms and for which it is desired to detect relative abundances. For example, RNA analyte 321 includes splice isoform 324, while RNA analyte 321′ includes different splice isoform 324′, and it is desired to detect the abundance of RNA analyte 321 relative to that of RNA analyte 321′. As illustrated in FIG. 3B, sensing probe 300″ respectively is hybridized to these targets of interest at process 310′ (hybridize probes to targets of interest). More specifically, one copy of sensing probe 300″ may be hybridized to each of RNA analytes 311, 311′. In this example, each sensing probe 300″ includes capture probe 301″ including a sequence that is complementary to one or more exons in both of RNA analytes 311, 311′ and that terminates immediately prior to the splice isoform it is desired to detect and quantify. Each sensing probe 300″ also can include the same code 302″ as one another which can be coupled to a specific bead in a manner such as described with reference to FIGS. 1A-1C. In some examples, the RNA analytes (e.g., the splice isoforms that it is desired to detect) are detected via differences in fluorescence that are caused by differences between the analytes. Illustratively, at process 320′ (single-base extension with ffNs), the respective capture probes 301″ of the sensing probes 300 are each extended by a single base with ffNs that are fluorescently labeled. Because the sequences of RNA analytes 311, 311′ differ from one another by the splice isoform (e.g., exon3 or exon5), addition of differently fluorescently labeled ffNs to the location with that splice isoform result in different optical signals that may be distinguished from one another. The sensing probes can be coupled to one or more beads, e.g., to respective beads, optically detected, and the corresponding beads decoded in a manner such as described with reference to FIGS. 1A-1C (detect and decode on universal bead array).

In examples such as described with reference to FIGS. 2A-2F and 3A-3B, note that the ffN optionally may be fluorescently labeled after being added to the capture probe, rather than before being added to the capture probe. Additionally, or alternatively, the ffN may be coupled to multiple fluorophores so as to provide an amplified optical signal. Example methods for adding multiple fluorophores to a nucleotide are described in greater detail below with reference to FIGS. 7A-16E.

While certain examples of nucleotide analytes are described with reference to FIGS. 2A-2F and 3A-3B, the present sensing probes suitably may be adapted to selectively couple to any type of analyte, such as non-nucleotide analytes. Examples of non-nucleotide analytes include proteins and metabolites. Examples of sensing probes suitable for selectively coupling to non-nucleotide analytes include antibodies, such as described below with reference to FIGS. 4A-4B, or aptamers, such as described below with reference to FIGS. 5A-5C. Such sensing probes may be selectively coupled to beads, based upon which the analyte identity may be determined by decoding the beads in a manner such as described with reference to FIGS. 1A-1C.

For example, FIGS. 4A-4B schematically illustrate example antibody-based process flows for detecting protein analytes in a bead-based system. In the example illustrated in FIG. 4A, a solution may include a plurality of different proteins, and it may be desired to detect proteins 411, 411′ which are different than one another. At process 410 (general protein label), the proteins in the solution may be labeled with fluorophores 412 using a general protein dye (such as amine-reactive fluorophores or haptens). Nonlimiting examples of proteins 411, 411′ include kinases, serine hydrolases, metalloproteases, and disease-specific biomarkers such as antigens for specific diseases. This fluorescent labeling may be followed by in solution binding of sensing probes to enrich for the proteins of interest at process 420 (enrich for targets of interest). For example, sensing probe 400 may include antigen 413 which is specific to protein 411, and code 402 which is specific to a particular bead, and sensing probe 400′ may include antigen 413′ which is specific to protein 411′, and code 402′ which is specific to a particular bead. Antigen 413 may specifically bind protein 411, which may cause sensing probe 400 to become fluorescently labeled via fluorophore 412 coupled to protein 411. Antigen 413′ may specifically bind protein 411′, which may cause sensing probe 400′ to become fluorescently labeled via fluorophore 412′ coupled to protein 411′. Sensing probes 400, 400′ may be coupled to respective beads, and non-bound protein may be washed out. The fluorescence from fluorophores 412, 412′ may be respectively detected via imaging. Sensing probes 400, 400′ may be removed from the beads, a primer annealed to the beads, and the beads decoded in a manner such as described with reference to FIGS. 1A-1C (detect and decode on universal bead array) to identify the analytes that are respectively bound to the sensing probe. Note that all sensing probes in the solution may become coupled to respective beads, but only sensing probes that captured a protein will also generate a fluorescent signal.

In other examples, proteins are first captured by sensing probes to select targets of interest, prior to fluorescent labeling. In the example illustrated in FIG. 4B, a solution again may include a plurality of different proteins, and it may be desired to detect proteins 421, 421′ which are different than one another. At process 410′ (enrich for targets of interest), in-solution binding of sensing probes to enrich for the proteins of interest is performed. For example, sensing probe 430 may include antigen 423 which is specific to protein 421, and code 432 which is specific to a particular bead, and sensing probe 430′ may include antigen 423′ which is specific to protein 421′, and code 432′ which is specific to a particular bead. Antigen 423 may specifically bind protein 421, and antigen 423′ may specifically bind protein 421′. The bound proteins 421, 421′ then may be fluorescently labeled at process 420′ (detect binding with fluorescent antibody). For example, antibodies 424, 424′ coupled to fluorophores 442 may be respectively coupled to bound proteins 421, 421′ before or after sensing probes 430, 430′ are coupled to respective beads, and non-bound protein then may be washed out. The fluorescence from fluorophores 412, 412′ may be respectively detected via imaging. Sensing probes 430, 430′ may be removed from the beads, a primer annealed to the beads, and the beads decoded in a manner such as described with reference to FIGS. 1A-1C (detect and decode on universal bead array) to identify the analytes that are respectively bound to the sensing probes. Note that all sensing probes in the solution may become coupled to respective beads, but only sensing probes that captured a protein will also generate a fluorescent signal. Note that antibodies 424, 424′ may target different epitopes of the respective proteins than one another, so that both antibodies 423, 424 may be simultaneously bound to protein 411, and so that antibodies 423′, 424′ may be simultaneously bound to protein 411′. In examples such as described with reference to FIG. 4B, background fluorescence signal from non-specific binding of antigens to proteins may be suppressed by providing two independent antibody binding events to generate fluorescent signal, substantially increasing specificity.

Note that antigens coupled to codes in a manner such as described with reference to FIGS. 4A-4B may be or include barcoded antibodies such as commercially available from BioLegend, Inc. (San Diego, Calif.). In such barcoded antibodies, the 5′ end of the nucleic acid code used for sample identification (via bead binding and decoding such as described with reference to FIGS. 1A-1C) is covalently coupled to an antibody. The content of such barcoded antibodies may be customizable to provide detection of desired non-nucleotide analytes, such as proteins.

Other example process flows use sensing probes having aptamers to capture analytes. Aptamers may be considered to be antibodies that are made out of nucleic acid sequences, and can be used to capture proteins and small molecules (such as metabolites) with high specificity. For example, FIGS. 5A-5C schematically illustrate example aptamer-based process flows for detecting protein or metabolite analytes in a bead-based system.

For example, in a manner such as described with reference to FIG. 4A, a general protein fluorescent dye followed by in solution capture of target proteins with evolved aptamers may be used. In the example shown in FIG. 5A, at process 510 (general protein label), different proteins 511 are labeled with a general protein label such as described with reference to FIG. 4A. Nonlimiting examples of proteins 511 include kinases, serine hydrolases, metalloproteases, and disease-specific biomarkers such as antigens for specific diseases. At process 520 (enrich for targets of interest), the labeled proteins are mixed with sensing probes 500 which include codes 502 coupled to aptamers 503, optionally via linkage 504. The aptamer 503 (aptamer with target specificity) which is specific to protein 511 captures that protein, together with fluorophore 512 coupled to that protein. As such, sensing probe 500 becomes fluorescently labeled with specificity to protein 511. Sensing probe 500 may be specifically coupled to a bead, and non-bound protein may be washed out. The fluorescence from fluorophore 512 may be detected via imaging. Sensing probe 500 may be removed from the bead, a primer annealed to the bead, and the bead decoded in a manner such as described with reference to FIGS. 1A-1C (detect and decode on universal bead array) to identify the analyte that was respectively bound to the sensing probe. Note that aptamers 503 may be selected so as to be specific to the respective combination of a given protein 511 and the fluorophore 512 coupled to that protein. Alternatively, aptamers 503 may be selected so as to bind to respective region(s) of a given protein 511 that do not contain reactive amino acid residues that would be labeled with a fluorophore 512, so that fluorophore 512 may not interfere with binding between the aptamers and the proteins to which those aptamers are specific.

In other approaches, fluorescent read-out of analyte capture may be obtained by linking aptamer binding of the target analyte to a conformational change that introduces a fluorescent signal. Conformational changes in aptamers upon target binding is well documented, including the spinach aptamer and riboswitches. For example, the spinach aptamer, which causes the compound 3,5-difluoro-4-hydroxybenzylidene imidazolinone (DHFBI) to fluoresce upon binding, can be conjugated to additional riboswitches or aptamers that render Spinach inactive until they have also bound their respective ligand. An aptamer that has not bound its target will not be able to fluoresce. In the example shown in FIG. 5B, at process 520′ (enrich for targets of interest), analytes such as proteins or metabolites (or a mixture thereof) are mixed with sensing probes 500′ which include codes 502′ coupled to aptamers 503′, optionally via linkage 504′. The aptamer 503′ (aptamer with target specificity) which is specific to protein or metabolite 511′ captures that protein or metabolite which activates fluorophore 512′ (fluorescent transducer). As such, sensing probe 500′ becomes fluorescently labeled with specificity to protein or metabolite 511′. Sensing probe 500′ may be specifically coupled to a bead, and non-bound protein and metabolites may be washed out. The fluorescence from fluorophore 512′ may be detected via imaging. Sensing probe 500′ may be removed from the bead, a primer annealed to the bead, and the bead decoded in a manner such as described with reference to FIGS. 1A-1C (detect and decode on universal bead array) to identify the analyte that was respectively bound to the sensing probe.

In still other approaches, fluorescent read-out of analyte capture may be obtained by linking aptamer binding of the target analyte to a conformational change that reveals a moiety, such as an oligonucleotide sequence, that can bind a fluorophore. Only an aptamer that has bound its target will reveal the moiety, thus linking target binding specifically to fluorescent signal. In the example shown in FIG. 5C, at process 520″ (enrich for targets of interest), analytes such as proteins or metabolites (or a mixture thereof) are mixed with sensing probes 500′ which include codes 502″ coupled to aptamers 503″, optionally via linkage 504″. The aptamer 503″ (aptamer with target specificity) which is specific to protein or metabolite 511″ captures that protein or metabolite which reveals moiety 560 (binding site for fluorophore). At process 521, fluorophore 512″ may be coupled to moiety 560 via moiety 561 to which the fluorophore is coupled. Moiety 561 may, for example, include an oligonucleotide sequence that is complementary to an oligonucleotide sequence of moiety 560. As such, sensing probe 500″ becomes fluorescently labeled with specificity to protein or metabolite 511″. Sensing probe 500″ may be specifically coupled to a bead, and non-bound protein and metabolites may be washed out. The fluorescence from fluorophore 512″ may be detected via imaging. Sensing probe 500″ may be removed from the bead, a primer annealed to the bead, and the bead decoded in a manner such as described with reference to FIGS. 1A-1C (detect and decode on universal bead array) to identify the analyte that was respectively bound to the sensing probe.

Note that aptamers coupled to codes in a manner such as described with reference to FIGS. 5A-5C may be or include barcoded aptamers for proteins and small molecules such as commercially available from SomaLogic, Inc. (Boulder, Colo.). In such barcoded aptamers, the 5′ end of the nucleic acid code used for sample identification (via bead binding and decoding such as described with reference to FIGS. 1A-1C) is covalently coupled to an aptamer. The content of such barcoded aptamers may be customizable to provide detection of desired non-nucleotide analytes, such as proteins. For further details regarding aptamer designs, see Stojanovic et al., “Modular aptameric sensors,” J. Am. Chem. Soc. 126: 9266-9270 (2004), the entire contents of which are incorporated by reference herein. Protocols for generating aptamers to be used in conjunction with Spinach to create sensing complexes are described in Litke et al., “Developing fluorogenic riboswitches for imaging metabolite concentration dynamics in bacterial cells,” Methods in Enzymology, Volume 527, Chapter 14: 315-333 (2016), the entire contents of which are incorporated by reference herein. For examples of aptamers that are specific to small molecules, see Pfeiffer et al., “Selection and biosensor application of aptamers for small molecules,” Frontiers in Chemistry 4: 25 (2016), the entire contents of which are incorporated by reference herein. For examples of aptamers for cardiac biomarker detection, see Grabowska et al., “Electrochemical aptamers-based biosensors for the detection of cardiac biomarkers,” ACS Omega 3(9): 12010-12018 (2018), the entire contents of which are incorporated by reference herein.

Note that the present sensing probes may include any suitable functionality for capturing analytes with specificity, and are not limited to aptamers, antigens, or oligonucleotides such as exemplified elsewhere herein. For example, the present sensing probes may include peptide or protein ligands that may be used to capture protein analytes with specificity. An example engineered peptide for capturing human serum albumin is described in Ogata et al., “Virus-enabled biosensor for human serum albumin,” Analytical Chemistry 89(2): 1373-1381 (2017), the entire contents of which are incorporated by reference herein. An example engineered peptide for capturing a prostate-specific membrane antigen is described in Arter et al., “Virus-polymer hybrid nanowires tailored to detect prostate-specific membrane antigen,” Analytical Chemistry 84: 2776-2783 (2012), the entire contents of which are incorporated by reference herein. Example peptide ligand libraries for detecting cancer biomarkers are described in Boschetti et al., “Protein biomarkers for early detection of diseases: The decisive contribution of combinatorial peptide ligand libraries,” Journal of Proteomics 188: 1-14 (2018), the entire contents of which are incorporated by reference herein.

In addition to detecting different analytes, in some circumstances it also may be useful to quantify the relative or absolute amounts of such analytes. One example approach to address this is to incorporate a measurement of total available binding sites. For example, FIGS. 6A-6C schematically illustrates example schemes for quantifying analyte concentrations in a bead-based system. The example shown in FIG. 6A is similar to the example illustrated in FIG. 4A, in that proteins 611 may be labeled with fluorophores 612 using a general protein and captured by sensing probe 600 including antigen 613 which is specific to protein 611 and code 602 which is specific to a particular bead. The binding of protein 611 by antigen 613 causes sensing probe 600 to become fluorescently labeled via fluorophore 612 coupled to protein 611. Additionally, each sensing probe 600 includes fluorophore 614. The sensing probes 600 may be specifically coupled to beads in a manner such as described with reference to FIGS. 1A-1C, and the fluorescence from fluorophores 612, 614 may be respectively detected via imaging. The fluorescence from fluorophores 614 (signal representing all possible binding sites) indicates total available antibodies coupled to each bead, and the fluorescence from fluorophores 612 (signal representing analyte binding) indicates the antibodies that captured protein 611. The fluorescence from fluorophores 612 may be scaled using at least (e.g., divided by) the fluorescence from fluorophores 614 to calculate or estimate the relative or absolute amount of captured protein 611. The fluorescence from fluorophores 612 also or alternatively may be used to help normalize across bead types, for example if one capture bead happens to have higher capture efficiency.

The example shown in FIG. 6B is similar to the example illustrated in FIG. 5A, in that proteins 611′ may be labeled with fluorophores 612′ using a general protein and captured by sensing probe 600′ including aptamer 603 which is specific to protein 611′ and code 602′ which is specific to a particular bead. The binding of protein 611′ by aptamer 603 causes sensing probe 600′ to become fluorescently labeled via fluorophore 612′ coupled to protein 611′. Additionally, each sensing probe 600′ includes fluorophore 614′. The sensing probes 600′ may be specifically coupled to beads in a manner such as described with reference to FIGS. 1A-1C, and the fluorescence from fluorophores 612′, 614′ may be respectively detected via imaging. The fluorescence from fluorophores 614′ (signal representing all possible binding sites) indicates total available antibodies coupled to each bead, and the fluorescence from fluorophores 612′ (signal representing analyte binding) indicates the antibodies that captured protein 611′. The fluorescence from fluorophores 612′ may be scaled using at least (e.g., divided by) the fluorescence from fluorophores 614′ to calculate or estimate the relative or absolute amount of captured protein 611′. The fluorescence from fluorophores 612′ also or alternatively may be used to help normalize across bead types, for example if one capture bead happens to have higher capture efficiency.

As an alternative to, or in addition to, detecting abundance of an analyte, activity of the analyte may be detected by using a molecule in place of the aptamer or antibody (which recognizes an epitope on the protein, optionally in both active and inactive forms). That molecule may be a substrate mimic for an enzyme, in which the molecule binds in the active site and forms a covalent bond. For example, the molecule may be a non-hydrolyzable analog of the natural enzyme substrate in a manner such as described for serine hydrolases in Liu et al., “Activity-based protein profiling: The serine hydrolases,” PNAS 96(26): 14694-14699 (1999); or for metalloproteases in Saghatelian et al., “Activity-based probes for the proteomic profiling of metalloproteases,” PNAS 101(27): 10000-10005 (2004); the entire contents of both of which are incorporated by reference herein. As a result, while both active and inactive forms of the enzyme are detected and quantified using aptamers/antibodies, only active forms may be detected with the activity-based probe. These probes may also or alternatively be used to provide a handle with which to use an aptamer/antibody, or a molecule such as streptavidin.

In some examples, multiple sensing probes may be expected to end up coupled to the same bead as each other, even if those sensing probes capture different analytes than one another. For example, different nucleotide analytes (SNPs such as A and G in FIG. 2A; methylations such as Me-C and C in FIG. 2B; methylations such as Me-C and C in FIG. 2C; or RNA splice isoforms such as exon3 and exon5 in FIG. 3B) may be captured by the same type of sensing probe as one another, and may be fluorescently labeled differently than one another due to differences between the analytes. Or, for example, different non-nucleotide analytes may be captured by the same type of sensing probe as one another, and may be fluorescently labeled differently than one another due to differences between the analytes. The differences between levels of fluorescence from different fluorophores coupled to a given bead may be used to obtain quantitative information about the relative amounts of the different analytes that had been captured by the sensing probes coupled to that bead. For example, the relative levels of signal may reflect the overall biology of the sample.

In the example shown in FIG. 6C (sensing multiple fluorophore signal intensity per bead allows quantification of ratiometric data types), a given bead is configured to hybridize to a single type of sensing probe that may capture different analytes (bead for a single target hybridized to capture probes). In panel (A) of FIG. 6C, fluorescence from only a single type of fluorophore (e.g., “blue”) is measured from that bead (signals measured). For an example interpretation for DNA SNP assay, such as described with reference to FIG. 2A, a 100% blue signal from the bead may be interpreted as meaning that the sample was homozygous for the genotype at the locus at which the blue fluorophore became coupled. For an example interpretation for DNA methylation assay, such as described with reference to FIG. 2B or 2C, a 100% blue signal from the bead may be interpreted as meaning that the sample was 100% methylated at the locus at which the blue fluorophore became coupled. For an example interpretation for RNA splice junction assay, such as described with reference to FIG. 3B, a 100% blue signal from the bead may be interpreted as meaning that the sample contained 100% of splice junction 1 at the locus at which the blue fluorophore became coupled.

In comparison, in panel (B) of FIG. 6C, fluorescence from multiple types of fluorophores (e.g., “red” and “blue”) is measured from a given bead (signals measured). For an example interpretation for DNA SNP assay, such as described with reference to FIG. 2A, a 50% blue signal and 50% red from the bead may be interpreted as meaning that the sample was heterozygous for the genotype at the locus at which the red and blue fluorophores became coupled. For an example interpretation for DNA methylation assay, such as described with reference to FIG. 2B or 2C, a 50% blue signal and 50% red signal from the bead may be interpreted as meaning that the sample was 50% methylated at the locus at which the blue and red fluorophores became coupled. For an example interpretation for RNA splice junction assay, such as described with reference to FIG. 3B, a 50% blue signal and 50% red signal from the bead may be interpreted as meaning that the sample contained 50% of splice junction 1 and 50% of splice junction 2 at the location to which the blue and red fluorophores became coupled. It will be appreciated that any suitable number and color of fluorophores may become coupled to any suitable beads, so long as the respective fluorescence from those fluorophores may be distinguished from one another, and that the relative levels of fluorescence from those fluorophores may be used to quantify the relative amounts of analytes in a sample, e.g., nucleotide analytes or non-nucleotide analytes.

Additionally, increasing sensitivity of analyte detection can be beneficial. For example, it may be more challenging to detect relatively rare analytes using labels that include only a single fluorophore, as compared to using labels that include multiple fluorophores. Example labels, and example methods of coupling labels with multiple fluorophores to nucleotides, analytes, sensing probes, or other chemical entities, are provided further below with reference to FIGS. 7A-16E.

### Amplifying Optical Detection of Analytes Using Multiple Fluorophores

Technologies that use fluorescent labels to detect analytes, such as nucleotides, may be limited by signal intensity, uniformity, and linear dynamic range. These include sequencing applications where low signal intensity may become an issue, particularly as feature sizes in flow cells become smaller, resulting in a decrease in the number of sequencing templates per cluster. Another example is genotyping array platforms where detection of a low number of molecules captured per bead would benefit from enhancement in signal relative to a single fluorescent labeling event. For certain applications such as detecting methylation, identifying copy number variations, or measuring RNA abundance (e.g., as described above with reference to FIGS. 2A-3B) a large linear dynamic range may be useful. Other examples include on-flow cell applications such as single molecule sequencing, spatial transcriptomics, or multi-omics (e.g., as described above with reference to FIGS. 2A-6B), where a relatively high level of signal amplification or a relatively large dynamic range, or both, may be useful. Provided herein are several example methods for using multiple fluorophores to amplify the optical detection of analytes. Such methods optionally may be utilized in conjunction with the bead-based system and methods for optically detecting multiple analytes such as described elsewhere herein. However, it will be appreciated that the present methods for amplifying optical detection using multiple fluorophores are not limited thereto, and suitably may be adapted to couple multiple fluorophores to any desired element.

FIGS. 7A-7D schematically illustrate example process flows for labeling an analyte with multiple fluorophores in a bead-based system. In some examples, the bead-based system illustrated in FIGS. 7A-7D may be similar to that described with reference to FIGS. 1A-6B. For example, FIG. 7A illustrates bead 760 including substrate 761 and oligonucleotide 762 which may include code and primer regions in a manner such as described with reference to FIG. 1B. Sensing probe 700 may include an oligonucleotide, which may include a capture probe and code region in a manner such as described with reference to FIG. 1A or 2A-6B. The capture probe may be coupled to a plurality of fluorophores 712, e.g., as a result of capturing an analyte in a manner such as described with reference to FIG. 1A or 2A-6B. At process 710 illustrated in FIG. 7A, sensing probe 700 may be coupled to bead 760 in a manner such as described with reference to FIG. 1B. The plurality of fluorophores 712 can amplify optical detection of sensing probe 700, e.g., as bound to bead 760, and thus enhance detection of an analyte that was captured by the sensing probe.

While fluorophores may be coupled to oligonucleotides or other sensing probes prior to those oligonucleotides being coupled to a bead, e.g., as shown in FIG. 7A, fluorophores also may be coupled to oligonucleotides after the oligonucleotides are coupled to beads. For example, FIG. 7B illustrates bead 760 including substrate 761 and oligonucleotide 762 which may include code and primer regions in a manner such as described with reference to FIG. 1B. Sensing probe 700′ may include an oligonucleotide, which may include a capture probe and code region in a manner such as described with reference to FIG. 1A or 2A-6B. The capture probe may be coupled to a moiety 711, e.g., as a result of capturing an analyte in a manner such as described with reference to FIG. 1A or 2A-6B. At process 710′ illustrated in FIG. 7B, sensing probe 700′ may be coupled to bead 760 in a manner such as described with reference to FIG. 1B. At process 720 illustrated in FIG. 7B, a plurality of fluorophores 712′ may be coupled to moiety 711. The plurality of fluorophores 712′ can amplify optical detection of sensing probe 700′, e.g., as bound to bead 760, and thus enhance detection of an analyte that was captured by the sensing probe.

In still other examples, fluorophores may be coupled to beads rather than to oligonucleotides or other sensing probes. For example, FIG. 7C illustrates bead 760 including substrate 761 and oligonucleotide 762 which may include code and primer regions in a manner such as described with reference to FIG. 1B. Sensing probe 700″ may include an oligonucleotide, which may include a capture probe and code region in a manner such as described with reference to FIG. 1A or 2A-6B. The capture probe optionally may be coupled to an analyte in a manner such as described with reference to FIG. 1A or 2A-6B. At process 710″ illustrated in FIG. 7C, sensing probe 700″ may be coupled to bead 760 in a manner such as described with reference to FIG. 1B. At process 720′ illustrated in FIG. 7C, nucleotide 730 coupled to a plurality of fluorophores 712″ may be coupled to oligonucleotide 762, e.g., using at least the sequence of the oligonucleotide of sensing probe 700″. The plurality of fluorophores 712″ can amplify optical detection of bead 760.

While fluorophores may be coupled to nucleotides prior to those nucleotides being coupled to a bead, e.g., as shown in FIG. 7C, fluorophores also may be coupled to nucleotides after the nucleotides are coupled to beads. For example, FIG. 7D illustrates bead 760 including substrate 761 and oligonucleotide 762 which may include code and primer regions in a manner such as described with reference to FIG. 1B. Sensing probe 700″ may include an oligonucleotide, which may include a capture probe and code region in a manner such as described with reference to FIG. 1A or 2A-6B. The capture probe optionally may be coupled to an analyte in a manner such as described with reference to FIG. 1A or 2A-6B. At process 710″ illustrated in FIG. 7D, sensing probe 700″ may be coupled to bead 760 in a manner such as described with reference to FIG. 1B. At process 720″ illustrated in FIG. 7D, nucleotide 730′ coupled to moiety 711′ may be coupled to oligonucleotide 762, e.g., using at least the sequence of the oligonucleotide of sensing probe 700″. At process 740 illustrated in FIG. 7D, sensing probe 700″ may be dehybridized from bead 760. At process 750 illustrated in FIG. 7D, a plurality of fluorophores 712″ may be coupled to moiety 711′. The plurality of fluorophores 712″ can amplify optical detection of bead 760.

It should be appreciated that in examples such as described with reference to FIGS. 7A-7D, any suitable nucleotide or oligonucleotide may be coupled to a plurality of fluorophores and then coupled to a bead. Additionally, the oligonucleotide is not limited to being a sensing probe and is not required to have captured an analyte.

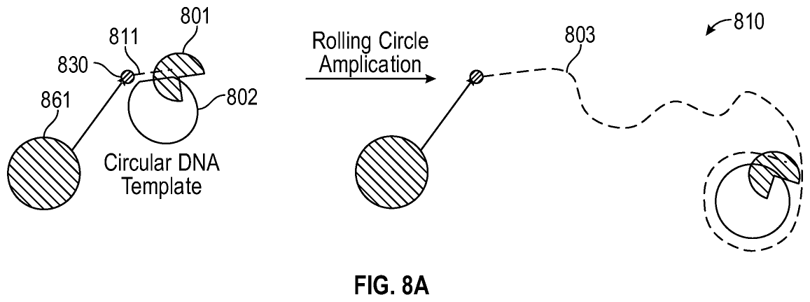

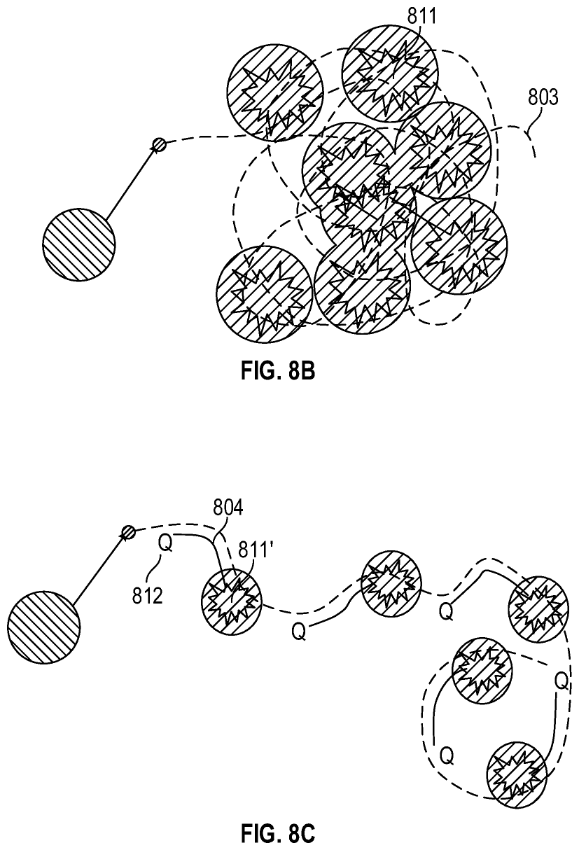

Multiple fluorophores may be added to nucleotides, oligonucleotides, sensing probes, beads, or any other suitable element using any of a variety of methods. Examples of these methods are provided with reference to FIGS. 8A-16E, but will be appreciated that other suitable methods readily may be envisioned.

FIGS. 8A-8C schematically illustrate example process flows for using rolling circle amplification (RCA) to label an analyte, such as a nucleotide, with multiple fluorophores in a bead-based system. In FIG. 8A, nucleotide 830 coupled to moiety 811 is coupled to substrate 861 of a bead in a manner similar to that described with regard to FIG. 7D. Moiety 811 may be or include an oligonucleotide primer. Processive polymerase 801 is configured to bind the oligonucleotide primer and circular DNA template 802, and to extend the primer using at least the sequence of the circular DNA template using RCA. For example, at process 810 (rolling circle amplification), the RCA generates an elongated, repeated sequence 803 using at least the sequence of circular DNA template 802. The repeated sequence may include a plurality of repeated portions that can be respectively coupled to fluorophores. Such coupling may be non-specific to the repeated portions. For example, as illustrated in FIG. 8B, a plurality of fluorescently labeled DNA intercalators may be coupled to elongated, repeated sequence 803. The use of non-specific intercalators may, for example, include four wells to measure incorporation of the four different nucleotides, followed by washing of excess, followed by addition of RCA reagents and comparison of which generates product. Alternatively, such coupling may be specific to the repeated portions. For example, as illustrated in FIG. 8C, a plurality of oligonucleotides 804, each including fluorophore 811′ and quencher (Q) 812 may be hybridized to the repeated portions and may act as molecular beacons. Optionally, such oligonucleotides 804 may be introduced as hairpins that unfold when brought sufficiently close to respective portions of sequence 803. It will be appreciated that any suitable element may be coupled to oligonucleotide primer 811, e.g., an analyte, sensing probe, oligonucleotide, bead, or other element besides a nucleotide, so as to label such element with a plurality of fluorophores in a manner so as to amplify optical detection of that element.

In examples such as described with reference to FIGS. 8A-8C, different nucleotides 803 may be optically distinguished from one another by providing oligonucleotide primers that are different than one another, as well as circular DNA templates than one another. Depending on the particular nucleotide 830 (and thus the particular oligonucleotide 801 coupled thereto), processive polymerase 801 may bind a particular one of the circular DNA templates 802 and thus generate a particular elongated, repeated sequence 803 that based upon which the particular nucleotide may be uniquely identified. For example, the elongated, repeated sequences 803 corresponding to different nucleotides may interact with different fluorescently labeled DNA intercalators than one another, or there may be incorporation of fluorescent nucleotides within the RCA product (specific to a template), thus providing different fluorescent labeling in a manner similar to that described with reference to FIG. 8B. Or, for example, the elongated, repeated sequences 803 corresponding to different nucleotides may interact with different oligonucleotides 804 (e.g., molecular beacons) than one another, providing different fluorescent labeling in a manner similar to that described with reference to FIG. 8C. For further details regarding coupling fluorophores to RCA products, see the following references, the entire contents of each of which are incorporated by reference herein: Krieg et al., “G-quadruplex formation in doubles strand DNA probed by NMM and CV fluorescence,” Nucleic Acids Research 43(16): 7961-7970 (2015); Li et al., “Dual functional Phi29 DNA polymerase-triggered exponential rolling circle amplification of target DNA embedded in long-stranded genomic DNA,” Scientific Reports 7: 6263 (2017); Le et al., “Direct incorporation and extension of a fluorescent nucleotide through rolling circle DNA amplification for the detection of microRNA 24-3P,” Bioorganic & Medicinal Chemistry Letters 28(11): 2035-2038 (2018); and Ali et al., “Rolling circle amplification: a versatile tool for chemical biology, materials science and medicine,” Chem. Soc. Rev. 43(10): 3324-41 (2014).

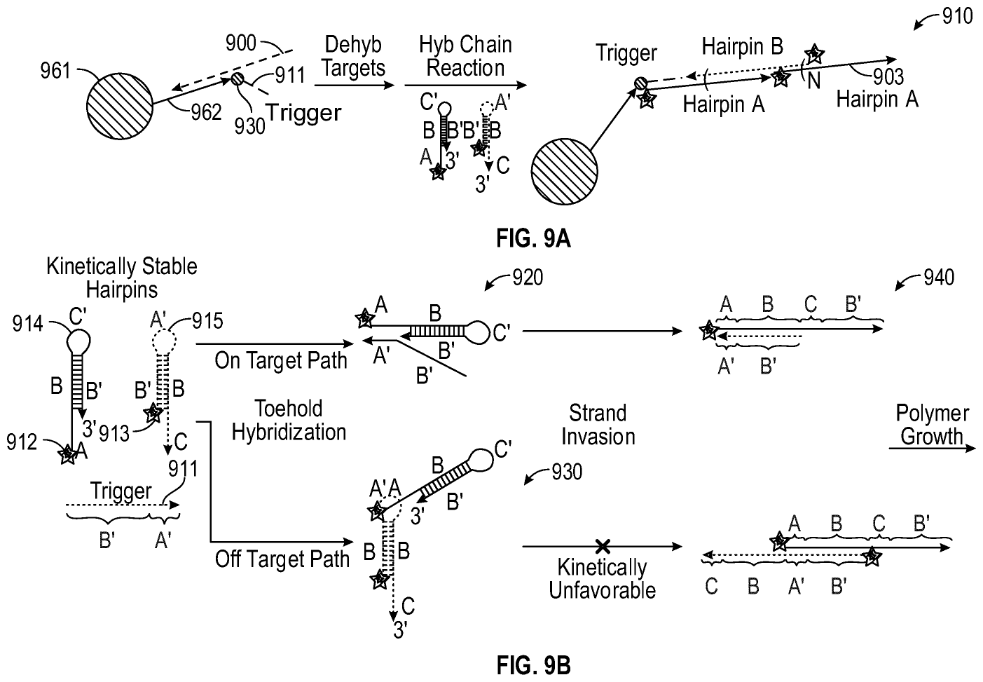

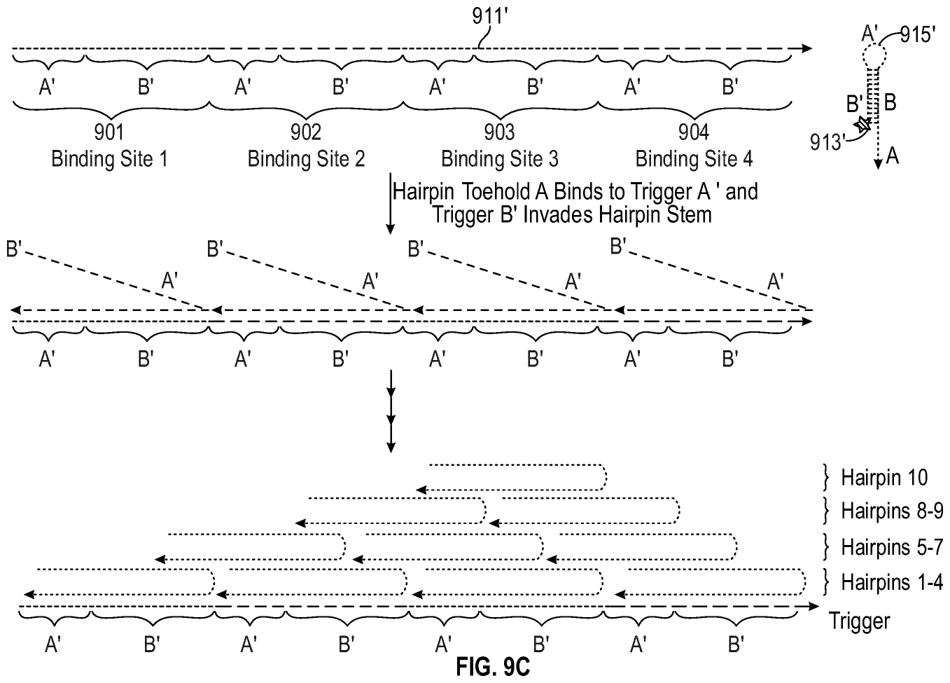

In still other examples, the hybridization chain reaction (HCR) is used to couple a plurality of fluorophores to an analyte, such as a nucleotide. This method can make use of a moiety such as a “trigger” oligonucleotide to initiate assembly of metastable hairpin oligonucleotides with complementary sequences. Such a method can be applied to a range of analyte detection schemes, such as nucleotide detection schemes. For example, FIGS. 9A-9C schematically illustrate example process flows for using a hybridization chain reaction (HCR) to label an analyte with multiple fluorophores.

In FIG. 9A, nucleotide 930 coupled to moiety 911 is coupled to substrate 961 of a bead in a manner similar to that described with regard to FIG. 7D. For example, oligonucleotide 900, e.g., a sensing probe, may be coupled to oligonucleotide 962 of the bead, and nucleotide 930 may be added to oligonucleotide 962 using at least the sequence of oligonucleotide 900. Moiety 911 may be or include an oligonucleotide primer, which may be referred to as a “trigger” oligonucleotide. Oligonucleotide 900 then may be dehybridized (dehyb targets) and HCR (hyb chain reaction) performed using a set of kinetically stable hairpins “A” and “B,” either or both of which are fluorescently labeled, to couple a plurality of fluorophores to nucleotide 930 by forming elongated sequence 903 which may be significantly longer than suggested in FIG. 9A. For example, in a manner such as elaborated in FIG. 9B, nucleotide 930 may be coupled to trigger oligonucleotide 911 which may include a first trigger sequence A′ and a second trigger sequence B′. A plurality of fluorophores may be coupled to nucleotide 930 by contacting trigger oligonucleotide 911 with a plurality of kinetically stable hairpins, e.g., a plurality of first oligonucleotide hairpins 914 and a plurality of second oligonucleotide hairpins 915. Each of the first oligonucleotide hairpins 914 includes a first fluorophore 912, a single-stranded toehold sequence A complementary to first trigger sequence A′, a first stem sequence B complementary to second trigger sequence B′, a second stem sequence B′ that is temporarily hybridized to first stem sequence B, and a single-stranded loop sequence C′ disposed between the first stem sequence B and the second stem sequence B′. Each of the second oligonucleotide hairpins includes a second fluorophore 913, a single-stranded toehold sequence C complementary to single-stranded loop sequence C′, a first stem sequence B complementary to second trigger sequence B′, a second stem sequence B′ that is temporarily hybridized to first stem sequence B, and a single-stranded loop sequence A′ disposed between the first stem sequence B and the second stem sequence B′.

As illustrated at process 920 in FIG. 9B (on target path toehold hybridization), responsive to hybridization of the single-stranded toehold sequence A of one of the first oligonucleotide hairpins 914 to first trigger sequence A′ of the trigger oligonucleotide 911, and at process 930 (off target path toehold hybridization) the second stem sequence B′ of that first oligonucleotide hairpin dehybridizes from the first stem sequence B of that first oligonucleotide hairpin. Subsequently, in strand invasion process 940, the single-stranded toehold sequence C of one of the second oligonucleotide hairpins 915 hybridizes to the single-stranded loop sequence C′ of that first oligonucleotide hairpin; and the second stem sequence B′ of that second oligonucleotide hairpin dehybridizes from the first stem sequence B of that second oligonucleotide hairpin. In a subsequent polymer growth process, responsive to hybridization of the single-stranded toehold sequence A of another one of the first oligonucleotide hairpins 914 to single-stranded loop sequence A′ of that second oligonucleotide hairpin 915, the second stem sequence B′ of that first oligonucleotide hairpin 914 dehybridizes from the first stem sequence B of that first oligonucleotide hairpin; the single-stranded toehold sequence C of another one of the second oligonucleotide hairpins 915 hybridizes to the single-stranded loop sequence of that first oligonucleotide hairpin; and the second stem sequence B′ of that second oligonucleotide hairpin 915 dehybridizes from the first stem sequence B of that second oligonucleotide hairpin. In such a manner, a plurality of first and second hairpins 914, 915, one or both of each of which may include a fluorophore, may become coupled to trigger oligonucleotide 911 and thus to bead substrate 961. In comparison, at off target path process 930, hybridization of toehold A of first hairpin 914 to single-stranded loop sequence A′ of second hairpin 915, prior to dehybridization of first hairpin stem sequences B, B′ from one another initiated by trigger nucleotide 911, results in kinetically unfavorable hybridization processes.

In methods such as described with reference to FIGS. 9A-9B, the specificity of signal generation may be based, in part, on the kinetic stability of the DNA hairpins. Hybridization of the trigger oligonucleotide to the toehold of one of the hairpins followed by strand invasion (repeated adding of first and second hairpins) yields a duplex with single stranded regions complementary to one another. An example benefit of using such HCR to couple multiple fluorophores to a nucleotide (or other suitable element) is ease of use. For example, signal amplification by HCR can use a single reagent solution including a mixture of hairpin sequences, can be performed at room temperature, and does not require specialty reagents such as custom produced and covalently modified antibodies. Fluorescently labeled hairpin sequences are readily available from multiple commercial sources and are produced by routine methods. Additionally, HCR is an enzyme-free technique with a polymerase chain reaction-like level of sensitivity. Another example benefit of using such HCR to couple multiple fluorophores to a nucleotide (or other suitable element) is limited background. For example, HCR can assemble bright multiple-fluorophore structures from a single nucleation point (the trigger oligonucleotide) with specificity, whereas non-specific binding events may produce low background fluorescence relative to the self-assembled structures such as described with reference to FIGS. 9A-9B. This means that relatively high fluorescence intensity can be achieved without significant increases in background.

Other example benefits of using such HCR to couple multiple fluorophores to a nucleotide (or other suitable element) are specificity and tunability. For example, the use of oligonucleotides as signal generating moieties provides ease of customization. Illustratively, the sequence of the hairpin oligonucleotides may be modified to increase their kinetic stability or the rate of polymerization. The fluorescent properties of the hairpin oligonucleotides may be readily modified by including any of a wide range of commercially available fluorescent base modifications, alternative base modifications such as biotin or dinitrophenol that introduce affinity handles for additional signal generation schemes, or reactive sites such as amines or azides that can be used for post-synthetic modification. Because of the defined structure of the DNA double helix, the positioning of each of these modifications is known and can be used to prevent intermolecular self-quenching or to intentionally introduce interactions for FRET pairs or quenched dyes.

Another example benefit of using such HCR to couple multiple fluorophores to a nucleotide (or other suitable element) is extension of strategy for increased or defined signal generation. For example, an alternative implementation of HCR can be used to create defined supramolecular structures with relatively uniform numbers of fluorophores. Such an approach may be particularly useful when relative quantitation is desired. For example, FIG. 9C schematically illustrates another example process flow for using HCR to label an analyte, such as a nucleotide, with multiple fluorophores. In the example shown in FIG. 9C, trigger oligonucleotide 911′ includes a plurality of binding sites, e.g., binding site 1 901, binding site 2 902, binding site 3 903, and binding site 4 904 to which corresponding sequences of hairpin 915′ can hybridize. In a manner similar to that described with reference to FIG. 9B, oligonucleotide hairpin 915′ includes fluorophore 913′, a single-stranded toehold sequence A complementary to single-stranded sequence A′ of trigger 911′, a first stem sequence B complementary to single-stranded sequence B′ of trigger 911′, a second stem sequence B′ that is temporarily hybridized to first stem sequence B, a single-stranded loop sequence A′ disposed between the first stem sequence B and the second stem sequence B′ (hairpin toehold binds to trigger A′ and trigger B′ invades hairpin stem). Hybridization of toehold sequence A to single-stranded sequence A′ at any one of binding sites 1, 2, 3, or 4 of trigger 911′ causes strand invasion of trigger single stranded sequence B′ and hybridization to stem sequences B, displacing stem sequence B′. Then, this process repeats at the others of binding sites 1, 2, 3, 4 forming a layer including a plurality of hairpins (hairpins 1-4), as well as at hairpins that have already hybridized to trigger oligonucleotide 911′, generating three additional layers of hairpins (hairpins 5-7, hairpins 8-9, and hairpins 10).

It will be appreciated that any suitable element may be coupled to a trigger oligonucleotide for use in HCR, e.g., an analyte, sensing probe, oligonucleotide, bead, or other element besides a nucleotide, so as to label such element with a plurality of fluorophores in a manner so as to amplify optical detection of that element. Illustratively, a trigger oligonucleotide, via which multiple fluorophores can be coupled via HRC, can be covalently coupled to a protein target, detection body, or aptamer, such as described with reference to FIGS. 4A-5C. As one nonlimiting example, FIG. 10A schematically illustrates another example process flow for using a hybridization chain reaction (HCR) to label an analyte with multiple fluorophores. For example, in a manner similar to that described with reference to FIG. 4A, sensing probe 1000′ may include antigen 1013′ which specifically captures protein 1011′, and code 1002′ which is specific to a particular bead. However, instead of protein 1011′ being coupled to a fluorophore before being captured by sensing probe 1000′, protein 1011′ is labeled with moiety 1012′, which may be or include a trigger oligonucleotide such as described with reference to FIGS. 9A-9C. Additionally, or alternatively, in a manner similar to that described with reference to FIG. 4B, sensing probe 1000″ may include antigen 1013″ which specifically captures protein 1011″, and code 1002″ which is specific to a particular bead. Additionally, antibody 1014″ coupled to moiety 1012,″ which may be or include an trigger oligonucleotide such as described with reference to FIGS. 9A-9C, may be respectively coupled to bound protein 1011″. At process 1020′ (hybridization chain reaction), HCR is performed by sequentially coupling a plurality of fluorescently labeled hairpins to trigger oligonucleotide 1012′, to form elongated sequences having a plurality of fluorophores 1012′ via which sensing probe 1000′ or protein 1011′ may be detected. Similarly, at process 1020″ (hybridization chain reaction), HCR is performed by sequentially coupling a plurality of fluorescently labeled hairpins to trigger oligonucleotide 1012″, to form elongated sequences having a plurality of fluorophores 1012″ via which sensing probe 1000″ or protein 1011″ may be detected. Processes 1020′ and 1020″ optionally may be conducted in the same mixture as one another.

It will be appreciated that any suitable ligands may be used to couple moieties, such as trigger oligonucleotides, to elements to which it is desired to couple multiple fluorophores. For example, moieties such as trigger oligonucleotides may be conjugated to proteins via reactive protein ligands. FIG. 10B schematically illustrates example components that may be used in the process flow of FIG. 10A. In the example shown in FIG. 10B, moiety 1012′ (HCR trigger) may include reactive protein ligand 1050, linker 1052, and signal element 1054. In one specific example illustrated in FIG. 10B, reactive protein ligand 1050′ may include His-Tag, Spytag, maltose binding protein (MBP), linker 1052′ may include PEG groups of various lengths (PEG4, 8, 12, 24 etc.) or amino acid residues such as glycine, and signal element 1054′ may include an trigger oligonucleotide (oligo trigger) for signal amplification. It will be appreciated that protein conjugation may be achieved via classical methods such as amide formation, urea and thiourea formation, and reductive amination at Lys residues on the protein; or disulfide exchange, alkylation and conjugate addition to maleimides via Cys residues on the protein. In addition, there are more modern methods of conjugation such as 6π-Aza-electrocyclization reaction via Lys residues which may provide faster reaction kinetics for solvent accessible Lys residues. Similarly, a moiety such as an trigger oligonucleotide can be incorporated into an aptamer, and optionally may become available responsive to binding of a target analyte, following which HCR may be used to couple multiple fluorophores to that trigger oligonucleotide. Regardless of the particular reaction chemistry used, a protein, analyte, or other element can be covalently coupled to a moiety via which multiple fluorophores can be coupled, providing optical amplification for use in detecting that element.

In examples such as described with reference to FIGS. 9A-9C and 10A-10B, different analytes, such as nucleotides, may be optically distinguished from one another by providing oligonucleotide targets that are different than one another, as well as different hairpin oligonucleotides, than one another. For example, depending on the particular analyte, e.g., nucleotide, (and thus the particular trigger oligonucleotide coupled thereto), different fluorescently labeled hairpins may be coupled thereto, providing different fluorescent labeling to different analytes.

In other example approaches, signal amplification in bead based systems may make use of analytes, nucleotides, beads, sensing probes, or other elements of interest that are labeled with oligonucleotide primers that may be used for in situ synthesis of labels with multiple fluorophores in a spatially defined manner. For example, signal intensity may be increased by hybridizing the oligonucleotide primers to respective amplification templates, and enzymatically extending the amplification templates (e.g., using a suitable polymerase) in such a manner as to couple a plurality of fluorophores to the primer, and thus to the element of interest. In some examples, the spacing and type of fluorophores may be controlled using the amplification templates. Such controlled spacing may be used to inhibit intramolecular quenching. Such controlled type may be used to distinguish the elements of interest from one another, e.g., by coupling different fluorophores to different amplification templates. Additionally, in some examples, precision of intensity measurements may be increased by providing amplification templates that predefine the number of fluorophores that may be coupled thereto. As such, elongated labels including multiple fluorophores may be built from monomeric components, which may increase signal while retaining a similar level of noise (background) as from standard ffNs labeled with single fluorophores.

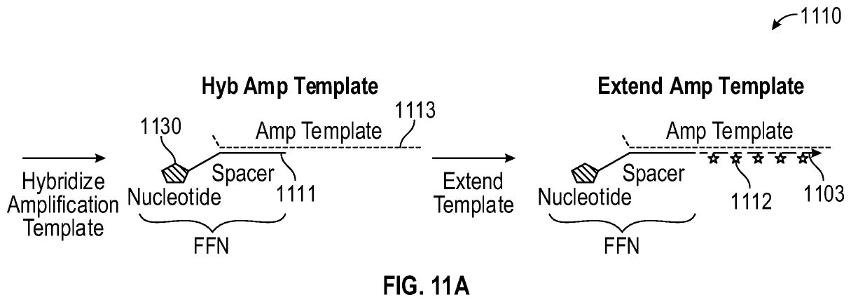

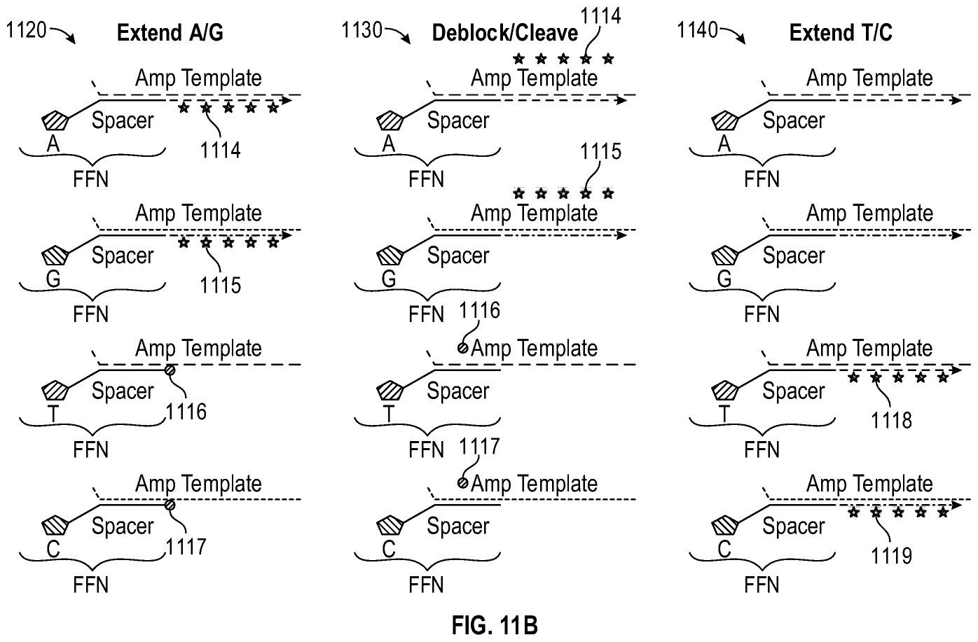

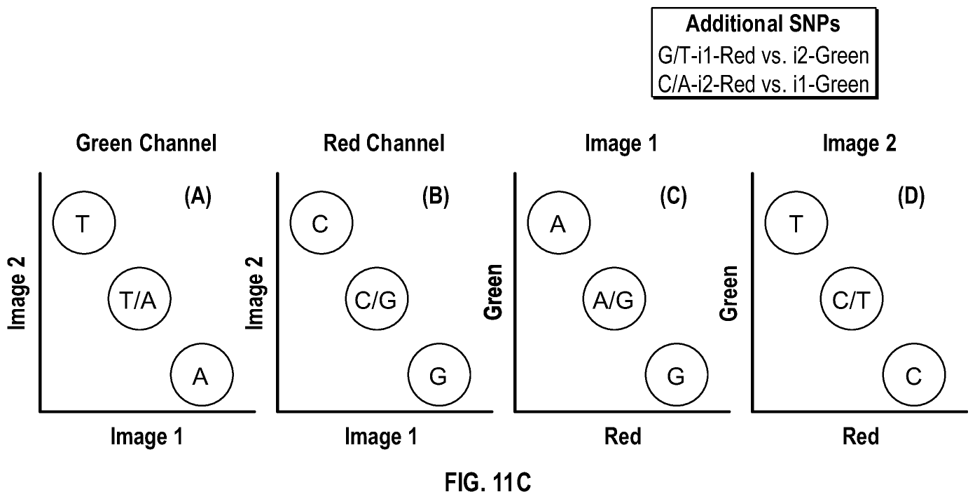

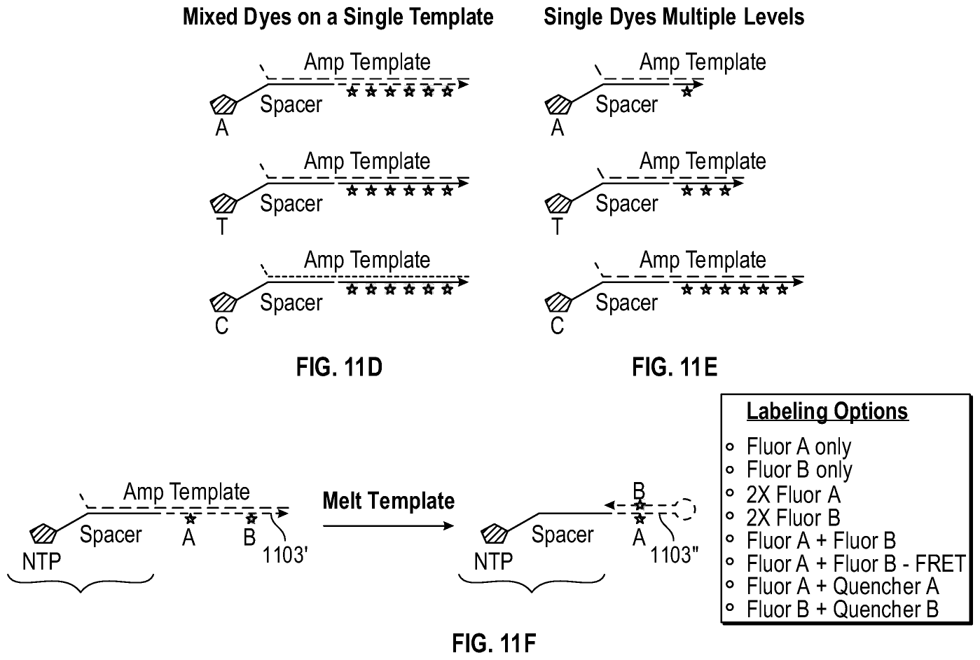

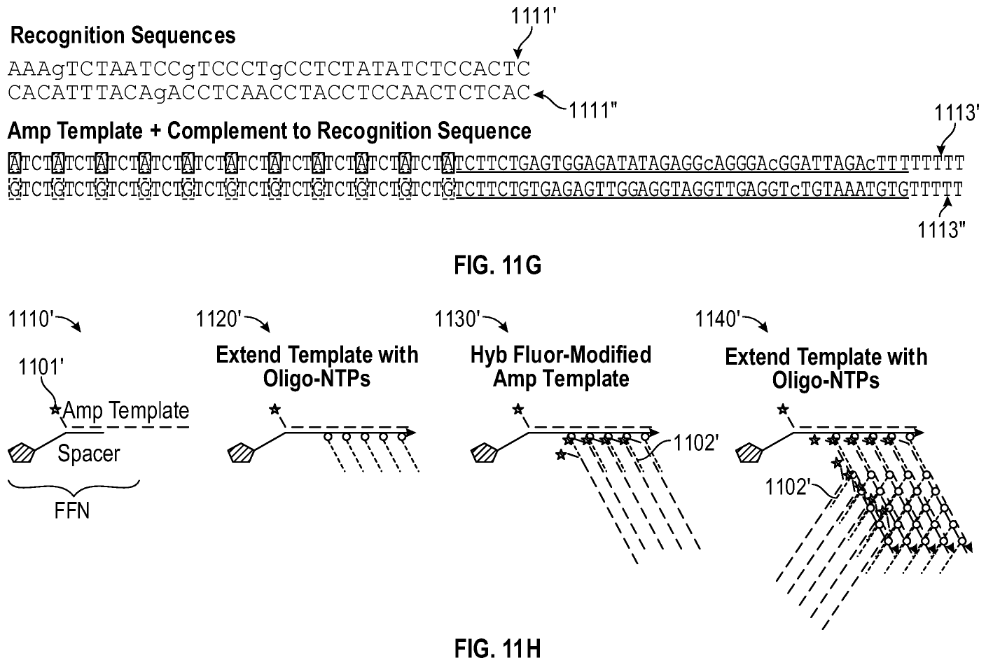

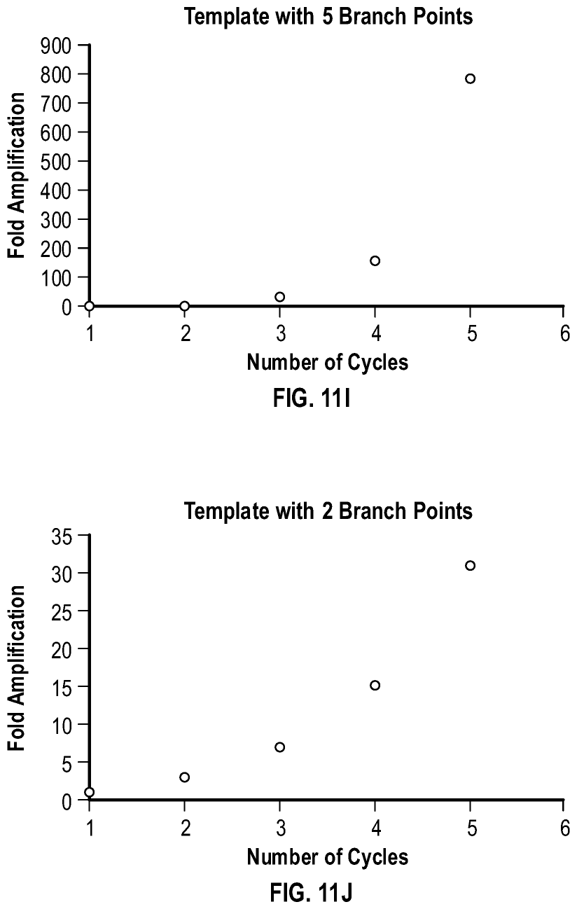

In some examples, nucleotides such as ddNTPs or 3′-blocked NTPs are modified to include respective oligonucleotide labels in a manner such as described below in the Working Examples section, and these oligonucleotide labels are used as primers to which amplification templates are respectively hybridized. For example, FIGS. 11A-11B schematically illustrate example process flows for using an amplification template to label an analyte with multiple fluorophores. In FIG. 11A, amplification template 1113 is hybridized to oligonucleotide primer 1111 of nucleotide 1130 (hyb amp template). Nucleotide 1130 may be an ffN such as indicated in FIG. 11A, or may be coupled to any suitable element, e.g., may be incorporated into or at a terminal end of a polynucleotide strand, for example coupled to a bead or to a sensing probe. At process 1110 (extend amp template), amplification template 1113 is used to extend oligonucleotide primer 1111 in such a manner as to synthesize elongated strand 1103 including multiple nucleotides that are coupled to respective fluorophores 1112. For example, nucleotide 1130 having oligonucleotide primer 1111 with amplification template 1113 hybridized to may be mixed with a solution of ffNs, some of which are labeled with single fluorophores. For example, one type of ffN in the solution may be labeled with one type of fluorophore, and another type of ffN in the solution may be labeled with another type of fluorophore. As the different ffNs are added to elongated strand 1103, fluorescently labeled ffNs are incorporated using at least the sequence of amplification template 1113. The particular sequence of elongated strand 1103, and thus the number, sequence, spacing, and types of fluorophores in elongated strand 1103 may be defined by the sequence of amplification template 1113. Different levels of and colors of fluorescence may be provided by tuning the length and sequence of amplification template 1113 so as to affect the number, density, and colors of fluorescently labeled nucleotides coupled thereto.

It will be appreciated that different nucleotides (or other elements) that it is desired to optically detect may have different oligonucleotide primers 1111 than one another, and thus may be hybridized to different amplification templates 1113 to which different numbers and types of fluorophores may be coupled in such a manner as to permit optically distinguishing the nucleotides or other elements from one another. Additionally, one or more of the oligonucleotide primers 1111 may be selectively blocked so as to further permit distinguishing the nucleotides (or other elements) from one another. For example, in FIG. 111B, at process 1120 (extend A/G) the amplification templates hybridized to the oligonucleotide primers of nucleotides A and G are used to generate elongated strands with different types of fluorophores 1114, 1115 relative to one another, while the oligonucleotide primers of nucleotides T and C are chemically blocked at 1116, 1117. The nucleotides then can be imaged so as to detect the A and G nucleotides. For example, as noted above, the nucleotides may be coupled to substrates such as beads (e.g., before or after process 1120), the beads coupled to a surface, and the beads imaged to detect fluorescence from the elongated strands. Because the A and G nucleotides have different fluorophores 1114, 1115 than one another, beads to which A is coupled may be optically distinguished from beads to which G is coupled, with high confidence because of the relatively large number of fluorophores coupled to each of those nucleotides.

At process 1130, deblock/cleave process removes the chemical blocks 1116, 1117 from T and C, and also cleaves fluorophores 1114, 1115 from the elongated strands coupled to A and G while otherwise leaving the elongated strands in place. At process 1140 (extend T/C), the amplification templates hybridized to the oligonucleotide primers of nucleotides T and C are used to generate elongated strands with different types of fluorophores 1118, 1119 relative to one another (these fluorophores may be the same as, or different than, the fluorophores that are used to label A and G), while the elongated strands previously coupled to nucleotides A and G at process 1120 inhibit any further addition of fluorophores to those nucleotides The nucleotides then can be imaged so as to detect the T and C nucleotides. For example, as noted above, the nucleotides may be coupled to substrates such as beads (e.g., before process 1140), the beads coupled to a surface, and the beads imaged to detect fluorescence from the elongated strands. Because the T and C nucleotides have different fluorophores 1118, 1118 than one another, beads to which T is coupled may be optically distinguished from beads to which C is coupled, with high confidence because of the relatively large number of fluorophores coupled to each of those nucleotides. Thus, via processes such as 1120, 1130, 1140, four different nucleotides (or other elements) may be optically distinguished from one another using the present amplification templates and two or more different fluorophores. Other processes using one fluorophore, two different fluorophores, three different fluorophores, or four different fluorophores readily may be envisioned. As one example, a cleavable linker may be provided between each nucleotide (or other element) and its oligonucleotide primer, or within the oligonucleotide primer, so as to permit selective cleavage of the entire elongated strand from that nucleotide in place of the fluorophore cleavage process 1130 described with reference to FIG. 111B.

FIG. 11C schematically illustrates an example scheme for four-element, e.g., four-base, discrimination that labels the elements with multiple fluorophores and uses an amplification template. In FIG. 11C, different elements (e.g., nucleotides A, T, C, and G) are fluorescently labeled using processes such as described with reference to FIG. 11B. Panel (A) in FIG. 11C corresponds to A/T discrimination, panel (B) corresponds to C/G discrimination, panel (C) corresponds to A/G discrimination, and panel (D) corresponds to C/G discrimination. A/C and G/T discrimination can be determined using at least both color and image differences. For example, additional SNPs such as G/T can be distinguished using at least differently colored fluorophores than one another (e.g., i1-red vs. i2-green), and C/A also can be distinguished using at least differently colored fluorophores than one another (e.g., i2-red vs. i1-green).

Any other suitable strategy for distinguishing elements, such as analytes (e.g., nucleotides) from one another, may be used. For example, FIGS. 11D-11F schematically illustrate example analytes labeled with alternative multiple fluorophores using an amplification template (amp template). In FIG. 11D (mixed dyes on a single template), different combinations of fluorophores may be added to elongated strands for different elements using at least sequences of respective amplification templates, permitting those elements to be optically distinguished from one another. In FIG. 11E (single dyes multiple levels), different numbers of fluorophores may be added to elongated strands for different elements using at least sequences of respective amplification templates, again permitting those elements to be optically distinguished from one another. In FIG. 11F, elongated strand 1103′ may include different labels “A” and “B” using at least the sequence of the amplification template. The template then may be dehybridized, responsive to which elongated strand 1103′ may form hairpin 1103″ having a structure using at least the sequence of the amplification template. Hairpin 1103″ may bring labels A and B in sufficient proximity to one another as to create an optically detectable signal. In various examples of labeling options shown in FIG. 11F, label A may be a fluorophore and may be the only fluorophore in the hairpin (fluor A only); label B may be a different fluorophore and may be the only fluorophore in the hairpin (fluor B only); both labels A and B may be the same fluorophore as one another (2× fluor A); both labels A and B may be a different fluorophore but the same fluorophore as one another (2× fluor B); labels A and B may both be in the hairpin and may be different than one another (fluor A+ fluor B); labels A and B may both be in the hairpin and may form a FRET pair (fluor A+fluor B−FRET); label A may be a fluorophore and label B may be a quencher for that fluorophore (fluor A+quencher A); or label A may be a different fluorophore and label B may be a quencher for that fluorophore (fluor B+quencher B).

FIG. 11G illustrates example sequences for use in a process flow for using an amplification template to label an analyte with multiple fluorophores. In non-limiting, purely illustrative examples, oligonucleotide primers (which also may be referred to as recognition sequences) 1111′ (SEQ ID NO:1), 1111″ (SEQ ID NO:2) may have different sequences than one another and may be coupled to different respective elements such as analytes (e.g., nucleotides) in a manner such as described elsewhere herein. Amplification templates 1113′, 1113″ (amp template+complement to recognition sequence) also may have different sequences than one another, e.g., may include underlined portions which respectively are complementary to, and hybridize to, oligonucleotide primers 1111′, 1111″. Additionally, amplification templates 1113′ (SEQ ID NO:3), 1113″ (SEQ ID NO:4) may have sequences designed to couple to different fluorescently labeled nucleotides than one another. For example, amplification template 1113′ may include the repeating sequence ATCT, and to the A of which fluorescently labeled T may be coupled in a repeating manner so as to provide an elongated strand including multiple fluorophores; while amplification template 1113″ may include the repeating sequence GTCT, and to the G of which fluorescently labeled C may be coupled in a repeating manner so as to provide an elongated strand including multiple fluorophores that are different than for the strand using at least template 1113′.

In some circumstances, it may be desired to provide tunable gain for sensing over a larger dynamic range. For example, for applications such as detecting analytes for which abundance may vary by multiple orders of magnitude, such as RNA, proteins, or metabolites, optical systems set for high sensitivity may experience saturation for targets with relatively high abundance, while optical systems set for low sensitivity may insufficiently detect targets with relatively low abundance. By implementing multiple cycles of amplification template hybridization and extension as provided herein, signal may be amplified exponentially and in a defined manner, enabling detection over a larger dynamic range. For example, FIG. 11H schematically illustrates an alternative example process flow for using an amplification template to label a nucleotide with multiple fluorophores. In FIG. 11H, at process 1110′ an amplification template (amp template) is hybridized to the oligonucleotide primer of an element, e.g., an analyte such as a nucleotide (FFN with optional spacer), in a manner such as described with reference to FIG. 11A. The amplification template may include fluorophore 1101′ to provide an initial low signal (e.g., for detecting high abundance analytes), and additional fluorophores may be added using subsequent processes to detect lower and lower abundance analytes.

For example, at process 1120′ of FIG. 11H (extend template with oligo-NTPs), the oligonucleotide primer is extended using at least the sequence of the amplification template. However, rather than incorporating fluorescently labeled nucleotides during process 1120′, nucleotides may be incorporated that include their own oligonucleotide primers, generating a branch point. At process 1130′ (hyb fluor-modified amp template), additional amplification templates may be hybridized to each of these oligonucleotide primers. Each of these amplification templates may include fluorophore 1102′ to provide an increased signal relative to that added at process 1110′ (e.g., for detecting lower abundance analytes), and additional fluorophores may be added using subsequent processes to detect still lower abundance analytes. At process 1140′ (extend template with oligo-NTPs), the oligonucleotide primers are extended using at least the sequence of the amplification template, e.g., either by incorporating fluorescently labeled nucleotides, or by incorporating nucleotides that include their own oligonucleotide primers to generate additional branch points. Further branch points may be generated by hybridizing additional amplification templates (which may be fluorescently labeled) to such oligonucleotide primers, followed by either by incorporating fluorescently labeled nucleotides, or by incorporating nucleotides that include their own oligonucleotide primers to generate additional branch points. As such, relatively large numbers of fluorophores may be coupled to elements, e.g., analytes such as nucleotides. FIGS. 11I-11J are plots illustrating example amplifications that may be obtained using the process flow of FIG. 11H. In FIG. 11I (templates with 5 branch points), an example amount of amplification that can be provided by using templates with five branch points as a function of the number of cycles (repetition of processes 1120′-1140′) is illustrated, and in FIG. 11J (templates with 2 branch points), an example amount of amplification that can be provided by using templates with two branch points as a function of the number of cycles (repetition of processes 1120′-1140′) is illustrated.

As such, approaches such as described with reference to FIGS. 11A-11J may provide for signal amplification that harnesses the sequence and structural tunability of oligonucleotides, as well as their high fidelity intra- and intermolecular interactions. Because of the molecular purity of the components of these systems, these approaches may achieve a relatively high degree of signal amplification while generating a similar or identical intensity of signal per initiation event and a relatively large dynamic range of intensity measurements. Additionally, these approaches may provide a relatively large number of possible combinations of fluorophores, quenchers, and FRET pairs for labeling elements, which may provide for multi-cycle incorporation followed by scanning that may reduce the number of fluidic and imaging cycles in SBS. In comparison, previously known antibody- or streptavidin-based sensing approaches may have some degree of heterogeneity in labeling efficiency and the number of binding events per signal amplification cycle may be poorly controlled.

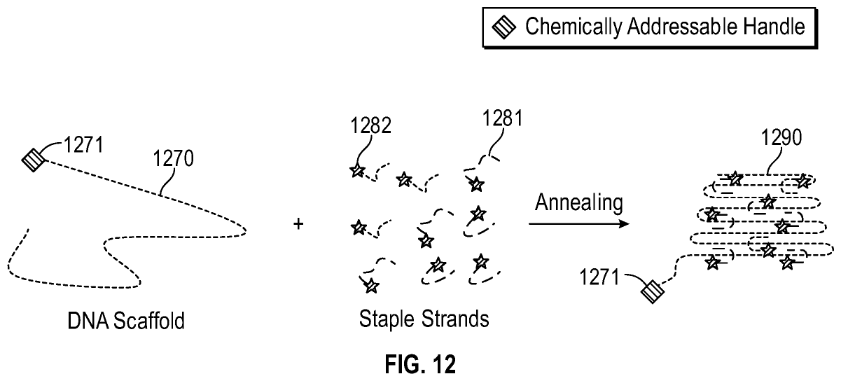

In still other examples, the multiple fluorophores may be coupled to a preformed, unitary structure that may be coupled to an element that it is desired to optically detect, e.g., an analyte such as a nucleotide. In some examples, the multiple fluorophores are provided in a “DNA origami,” referring to DNA with an intended tertiary structure, which also may be referred to as a supramolecular structure. FIG. 12 schematically illustrates an example process flow for using DNA origami to label an analyte with multiple fluorophores. DNA origami may be constructed by mixing a single long DNA molecule 1270, which may be referred to as a “template,” with short complementary sequences 1281 which may be called “staples” or “staple strands.” Each staple may bind to specific regions within the long DNA molecule and pull the long DNA molecule into a desired shape 1290, a nonlimiting example of which is illustrated in FIG. 12 (annealing). Each staple may have a unique sequence and may end up in a well-defined location in the final tertiary structure 1290. Because every staple optionally and independently may be individually functionalized, this allows for exact placement of specific functional elements, such as fluorophores 1282, on the tertiary structure 1290. Tertiary structure 1290 may include chemically addressable handle 1271, that may be coupled to an element that it is desired to optically detect, e.g., an analyte such as a nucleotide. Relatively large DNA origami structures may be formed from multiple, smaller DNA origami structures. For further details regarding DNA origami design and preparation, see the following reference, the entire contents of which are incorporated by reference herein: Wang et al., “The Beauty and Utility of DNA Origami,” Chem 2: 359-382 (2017).

In some examples, the DNA origami 1290 is directly coupled to an element, e.g., an analyte such as an ffN, via chemically addressable handle 1271 by biorthogonal conjugation chemistries such as copper(I)-catalyzed click reaction (between azide and alkyne), strain-promoted azide-alkyne cycloaddition (between azide and DBCO (dibenzocyclooctyne), or hybridization of an oligonucleotide to a complementary oligonucleotide. That element may be coupled to a substrate, such as a bead, in a manner such as illustrated in FIG. 7A or 7C. In other examples, the DNA origami 1290 is coupled to an element using a secondary labeling scheme. For example, a nucleotide may be incorporated into a polynucleotide (such as an oligonucleotide coupled to a bead or forming part of a sensing probe), and the DNA origami subsequently coupled to that nucleotide, e.g., in manner such as illustrated in FIG. 7B or 7D. Such an arrangement may be useful in situations where coupling the DNA origami to the nucleotide prior to incorporating the nucleotide to the polynucleotide may inhibit such incorporation, e.g., through steric effects. In various examples, the DNA origami 1290 is coupled to an already-incorporated nucleotide via chemically addressable handle 1271 using any suitable proteins, tags, or other specific interactions such as biotin-streptavidin, NTA-His-Tag, Spytag-Spycatcher, or hybridization of an oligonucleotide to a complementary oligonucleotide. Differentiation between elements, such as different nucleotides, may be achieved by selectively coupling to such elements different DNA origamis that may have different numbers, types, or combinations of fluorophores than one another in a similar manner as described with reference to FIGS. 11A-11F.

It should be appreciated that DNA origami may be useful for signal amplification for a variety of reasons. For example, DNA origami may be relatively easy to use. More specifically, DNA origami may be pre-assembled and may be easily customized to vary the supramolecule size, fluorophore identity, and location and number of fluorophores. As another example, DNA origami may provide relatively high signal uniformity. Because of the defined structure of the DNA origami, the positioning of fluorophores may be controlled and as a result, may be used to minimize intramolecular self-quenching or to promote FRET interactions. The controlled assembly of DNA origami may provide relatively high signal uniformity and reproducible intensities between uses. Additionally, DNA origami may provide specificity and tunability. For example, the fluorescent properties of DNA origami may be modified through a wide range of commercially available fluorophores, and a single chemically addressable handle such as amine, azide, TCO, tetrazine, DBCO, affinity handle (such as biotin), or oligonucleotide may be easily introduced during the synthesis of the DNA scaffold.

As noted elsewhere herein, it can be useful to increase the overall signal level in fluorescence based systems, such as for sequencing. For example, as the size of nanowells for performing sequencing on clusters decreases, so do the number of strands in in those clusters. The amount of signal may be increased by using relatively high intensity lasers to induce greater fluorescence. However, the energy from such lasers may damage DNA. Examples provided herein may incorporate features that reduce DNA damage and may increase fluorescence, while potentially simplifying incorporation of fluorescently labeled nucleotides into polynucleotides. As such, improved sequencing quality and improved modularity for ffN synthesis may be obtained.

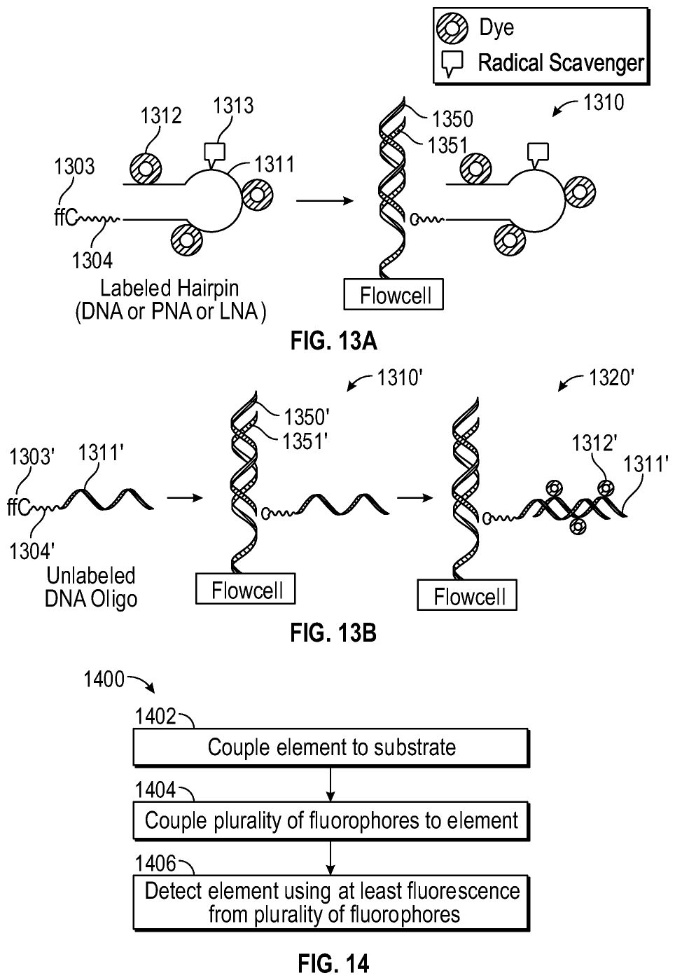

In some examples, a nucleotide may be labeled with an oligonucleotide in a manner similar to that described with reference to FIGS. 7D, 8A, 9A, and 11A. The oligonucleotide itself may include a plurality of fluorophores. For example, FIG. 13A schematically illustrates an example process flow for incorporating a DNA analyte labeled with a hairpin having multiple fluorophores into a polynucleotide. An ffN (e.g., ffC) 1303 may be coupled to oligonucleotide hairpin 1311 via optional linker 1304. Hairpin 1311 (labeled hairpin (DNA or PNA or LNA)) may include a plurality of fluorophores (dyes) 1312, and optionally one or more additional moieties 1313, such as an oxygen scavenger (radical scavenger). Optional linker 1304 may be used to increase the distance between ffN 1303 and hairpin 1311, e.g., may be a 30-mer or greater. Fluorophores 1312 may be added to hairpin 1311 in a separate reaction, and then coupled to linker 1304. PNA or LNA may be used as an alternative to DNA in hairpin 1311 for example, to alter stability and incorporation properties. At process 1310, ffN 1303 coupled to multiply fluorescently labeled hairpin 1311 is incorporated into first oligonucleotide 1350 using at least the sequence of second oligonucleotide 1351. Second oligonucleotide 1351 may be coupled to a substrate, such as a bead that may be located in a flow cell, or otherwise located in a flow cell. Thus, the multiple fluorophores 1312 become coupled to the substrate.

In other examples, the oligonucleotide to which the nucleotide is coupled (e.g., in a manner similar to that described with reference to FIGS. 7D, 8A, 9A, and 11A) is not fluorescently labeled, but may be fluorescently after incorporation of the nucleotide into a polynucleotide. For example, FIG. 13B schematically illustrates an example process flow for incorporating a DNA analyte coupled to a first oligonucleotide into a polynucleotide, followed by hybridizing to the first oligonucleotide to a second oligonucleotide with multiple fluorophores. In FIG. 13B, an ffN (e.g., ffC) 1303′ may be coupled to unlabeled oligonucleotide 1311′ (unlabeled DNA oligo) via optional linker 1304. Optional linker 1304 may be used to increase the distance between ffN 1303′ and oligonucleotide 1311′, e.g., may be a 30-mer or greater. At process 1310′, ffN 1303′ coupled to oligonucleotide 1311′ is incorporated into first oligonucleotide 1350′ using at least the sequence of second oligonucleotide 1351′. Second oligonucleotide 1351′ may be coupled to a substrate, such as a bead that may be located in a flow cell, or otherwise located in a flow cell. At process 1320′, oligonucleotide 1311″ labeled with multiple fluorophores 1312′ may hybridize with oligonucleotide 1311′ so as to couple those fluorophores to ffN 1303′, and to the substrate. Optionally, oligonucleotide 1311″ may include one or more additional moieties, such as an oxygen scavenger in a manner such as described with reference to FIG. 13A. A modular approach such as illustrated in FIG. 13B may provide ease of changing fluorophores and their positions and optical properties. In one specific implementation, the scheme illustrated in FIG. 13B may be modified to use heterodimeric protein coiled-coil motifs rather than DNA oligonucleotides. For example, in the configuration illustrated in FIG. 13B, oligonucleotide 1311′ may be replaced with a first coiled-coil, and oligonucleotide 1311″ may be replaced by a second coiled-coil that includes multiple fluorophores 1312′ and optionally one or more additional moieties, such as an oxygen scavenger. The second coiled-coil may interact with the first coiled-coil so as to couple the multiple fluorophores to the DNA analyte. For further details regarding coiled-coils and their interactions with one another, see Thomas et al., “A set of de novo designed parallel heterodimeric coiled coils with quantified dissociation constants in the micromolar to sub-nanomolar regime,” J. Am. Chem. Soc. 135(13): 5161-5166 (2013), and Crick, “The packing of α-helices: Simple coiled-coils,” Acta Cryst. 6: 689-697 (1953).

It will be appreciated that ffN designs such as described with reference to FIGS. 13A-13B may provide signal amplification due to increased number of fluorophores per ffN. Commercial oligonucleotide synthesis is well established and suited for installing fluorescently modified bases at specific locations and quantities within oligonucleotides. Selection of different oligonucleotide or hairpin lengths may control the distance between fluorophores so as to further enhance detection of the ffN.

Additionally, ffN designs such as described with reference to FIGS. 13A-13B may be expected to reduce or inhibit laser-induced DNA damage. For example, laser-induced DNA damage may be attributed to locally generated radical species which attack the proximal DNA. The use of extended linkers 1304, 1304′ and labeled oligonucleotides 1311, 1311″ may increase the distance between the DNA and the site where radicals are most likely to be generated. This, in turn, may reduce or inhibit the radical species from reaching and damaging the DNA on the substrate surface. Additionally, the hairpin oligonucleotide 1311 or hybridized oligonucleotide 1311″ may be expected to act as a shield or a scavenger for radical species, inhibiting these radicals from reaching DNA on substrate surface. Additional functionality, such as oxygen (radical) scavenging groups (e.g., COT (cyclooctatetraene) or methyl viologen) may be incorporated into hairpin oligonucleotide 1311 or hybridized oligonucleotide 1311″ to further inhibit DNA damage. It will be appreciated that such oxygen or radical scavenging groups may be incorporated into any other suitable elements described herein.

Accordingly, it will be appreciated that a wide variety of methods for coupling multiple fluorophores to an element are provided herein, via which optical detection of that element may be amplified. For example, FIG. 14 schematically illustrates an example process flow 1400 for detecting an analyte using at least multiple fluorophores. Process flow 1400 illustrated in FIG. 14 includes coupling an element to a substrate (process 1402). The element may include an analyte, such as a nucleotide analyte (such as a SNP, methylated nucleotide, or RNA) or a non-nucleotide analyte (such as a protein or metabolite), or may include a sensing probe, a nucleotide, or any other suitable element. Example structures that may be formed by coupling an element to a substrate are described with reference to FIGS. 7A-7D. In some examples, such as described with reference to FIGS. 1A-6B, the analyte may be coupled to a sensing probe, and the analyte may be coupled to the substrate via the sensing probe. In other examples, such as described with reference to FIGS. 8A-8C and 9A, the analyte may be coupled to an oligonucleotide that is coupled to the substrate. An example substrate is a bead, which may be free floating in solution or may be immobilized in a flow cell before or after process 1402.

Process flow 1400 illustrated in FIG. 14 includes coupling a plurality of fluorophores to the element (process 1404). In some examples, the plurality of fluorophores may be coupled to the element via the sensing probe. Illustratively, a plurality of fluorophores may be coupled to a sensing probe based upon that sensing probe having captured that element, e.g., in a manner such as described with reference to FIG. 10A. In other examples, the plurality of fluorophores may be coupled to the element via the substrate. Illustratively, a plurality of fluorophores may be coupled to an oligonucleotide coupled to a substrate based upon that substrate having been coupled to that element, e.g., in a manner such as described with reference to FIGS. 8A-8C and 9A.

The plurality of fluorophores may be coupled to the element before the element is coupled to the substrate, for example as described with reference to FIGS. 7A and 7C. Alternatively, the plurality of fluorophores may be coupled to the element after the element is coupled to the substrate, for example as described with reference to FIGS. 7B and 7D.

Process flow 1400 illustrated in FIG. 14 further includes detecting the element using at least fluorescence from the plurality of fluorophores (process 1406). The plurality of fluorophores provide enhanced fluorescence as compared to a single fluorophore.

Examples for performing process 1404 are provided throughout the present application. For example, the plurality of fluorophores may be coupled to the element using rolling circle amplification in a manner such as described with reference to FIGS. 8A-8C. The rolling circle amplification may generate an elongated, repeated sequence, and the plurality of fluorophores may be coupled to respective, repeated portions of that sequence. The fluorophores may be coupled to DNA intercalators that couple to the elongated, repeated sequence in a manner such as described with reference to FIG. 8B. Alternatively, the oligonucleotides may include fluorophores and quenchers hybridized to the repeated portions in a manner such as described with reference to FIG. 8C.

Alternatively, the element may be coupled to a trigger oligonucleotide to which a plurality of fluorescently labeled hairpins self-assemble in a manner such as described with reference to FIGS. 9A-9C or 10A-10B. The trigger oligonucleotide and hairpins may have sequences, and may interact with one another, in a manner such as described with reference to FIGS. 9A-9C or 10A-10B.

In other examples, the element may be coupled to an oligonucleotide primer, and coupling the plurality of fluorophores to the element may include hybridizing an amplification template to the oligonucleotide primer; and extending the oligonucleotide primer, using at least the amplification template, with a plurality of fluorescently labeled nucleotides to generate an extended strand including the plurality of fluorophores, in a manner such as described with reference to FIGS. 11A-11J. Optionally, at least one of the fluorophores is different than at least one other of the fluorophores, e.g., as described with reference to FIG. 11D. The method further may include dehybridizing the amplification template and forming the extended strand into a hairpin structure, e.g., as described with reference to FIG. 11F.

In still other examples, the element may be coupled to an oligonucleotide primer, and coupling the plurality of fluorophores to the element may include hybridizing an amplification template to the oligonucleotide primer; extending the oligonucleotide primer, using at least the amplification template, with a plurality of nucleotides that are respectively coupled to additional oligonucleotide primers; hybridizing additional amplification templates to the additional nucleotide primers; and extending the additional nucleotide primers, using at least the additional amplification templates, with a plurality of nucleotides that are either respectively coupled to fluorophores or are respectively coupled to further additional oligonucleotide primers, in a manner such as described with reference to FIGS. 11H-11J. The method optionally further includes hybridizing further additional amplification templates to the further nucleotide primers; and extending the additional nucleotide primers, using at least the additional amplification templates, with a plurality of nucleotides that are either respectively coupled to fluorophores or are respectively coupled to still further additional oligonucleotide primers, in a manner such as described with reference to FIGS. 11H-11J.

In yet other examples, the element is coupled to a DNA origami that includes the plurality of fluorophores, for example as described with reference to FIG. 12. Optionally, the DNA origami may include a combination of different fluorophores. In some examples, the element may be coupled to the DNA origami via copper(I)-catalyzed click reaction, strain-promoted azide-alkyne cycloaddition, hybridization of an oligonucleotide to a complementary oligonucleotide, biotin-streptavidin interaction, NTA-His-Tag interaction, or Spytag-Spycatcher interaction.

In still further examples, the element is coupled to an oligonucleotide, wherein the oligonucleotide includes the plurality of fluorophores, in a manner such as described with reference to FIGS. 13A-13B. Optionally, the oligonucleotide further includes a radical scavenger. The oligonucleotide may include a hairpin, e.g., as described with reference to FIG. 13A. Alternatively, the element may be directly coupled to a first oligonucleotide, and the first oligonucleotide may be hybridized to a second oligonucleotide that includes the plurality of fluorophores, e.g., as described with reference to FIG. 13B.

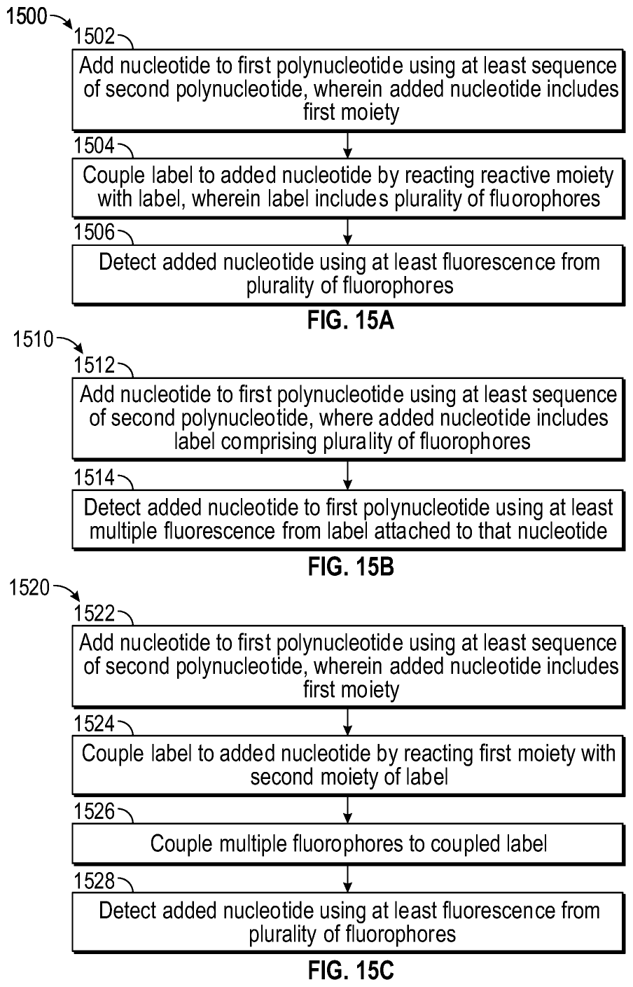

Although the present methods may be used to label any suitable elements with multiple fluorophores so as to amplify the elements' optical detection, an example element that is particularly useful to label with multiple fluorophores is a nucleotide. FIGS. 15A-15C schematically illustrate example process flows for detecting a nucleotide using at least multiple fluorophores. Example process flow 1500 illustrated in FIG. 15A includes adding a nucleotide to a first polynucleotide using at least a sequence of a second polynucleotide, wherein the added nucleotide includes a first moiety (process 1502). Example moieties are described elsewhere herein. Process flow 1500 illustrated in FIG. 15A includes coupling a label to the added nucleotide by reacting the first moiety with a second moiety of the label, wherein the label includes a plurality of fluorophores (process 1502). Process flow 1500 illustrated in FIG. 15A includes detecting the added nucleotide using at least fluorescence from the plurality of fluorophores (process 1504). Non-limiting examples of particular arrangements of elements that may be formed using processes 1502-1506 are provided with reference to FIGS. 7D, 12, and 13B.

Example process flow 1510 illustrated in FIG. 15B includes adding a nucleotide to a first polynucleotide using at least a sequence of a second polynucleotide, wherein the added nucleotide is coupled to a label includes a plurality of fluorophores (process 1512). Process flow 1510 also includes detecting the added nucleotide using at least fluorescence from the plurality of fluorophores (process 1514). Non-limiting examples of particular arrangements of elements that may be formed using processes 1512-1514 are provided with reference to FIGS. 7C and 13A.

Example process flow 1530 illustrated in FIG. 15B includes adding the nucleotide to a first polynucleotide using at least a sequence of a second polynucleotide, wherein the added nucleotide includes a first moiety (process 1522). Process flow 1530 also includes coupling a label to the added nucleotide by reacting the first moiety with a second moiety of the label (process 1524). Process flow 1530 also includes coupling multiple fluorophores to the coupled label (process 1526). Process flow 1530 also includes detecting the added nucleotide using at least fluorescence from the plurality of fluorophores (process 1528). Non-limiting examples of particular arrangements of elements that may be formed using processes 1522-1528 are provided with reference to FIGS. 8A-8C, 9A-9C, 10A-10B, and 11A-11J.

### Non-Limiting Working Examples

The following examples are purely illustrative, and not intended to be limiting.

Hybridization chain reaction (HCR) was used to amplify optical signals in bead-based genotyping, e.g., in which the analyte of interest was a SNP. As described below, HCR was found to increase signal by 8-30 fold depending on the sample input without any corresponding increase in background, and the same strategy was found to work with four unique trigger sequences and hairpin pairs on a standard whole-genome-amplified DNA sample and 10k-plex bead pool.

In order to implement HCR on Illumina flow cells using SBS polymerases, ddNTPs modified with 30-mer trigger oligonucleotides were synthesized using reaction schemes 1 and 2 shown below:

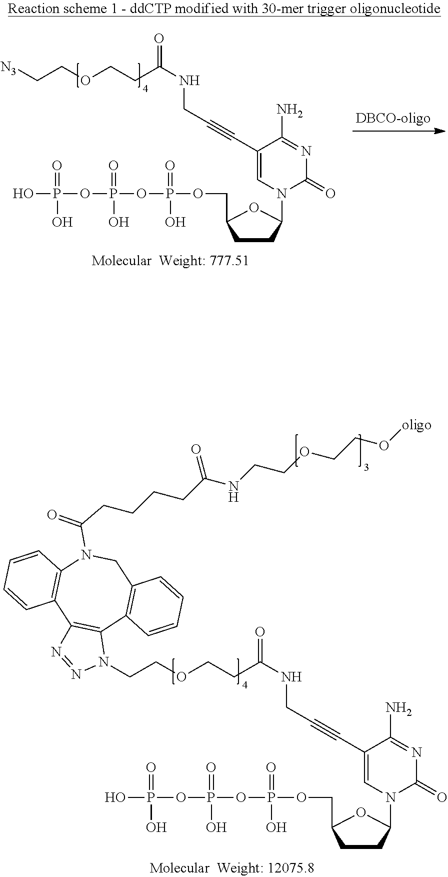

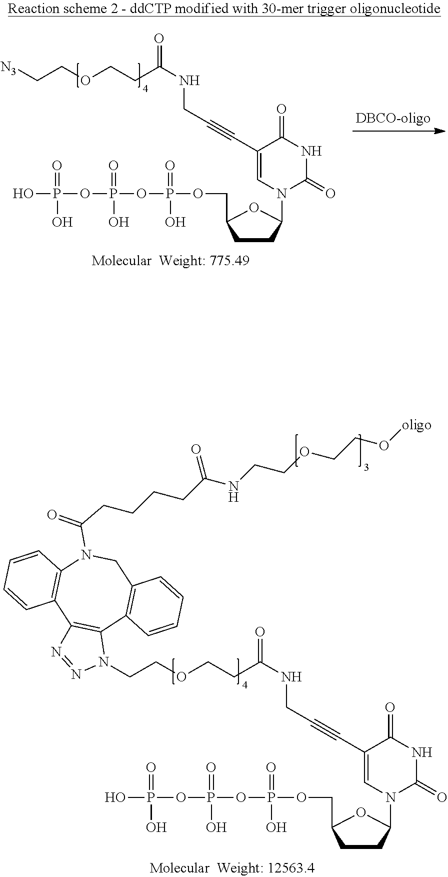

Each ddNTP-oligo conjugate was prepared by adding DBCO-oligo (1 eq, 5 mM) in water to ddNTP-PEG4-azide (1 eq, 5 mM) in 2×PBS (pH 7.4) and stirred at room temperature for 4 hours. The reaction mixture was purified on reversed phase C18 and eluted with a mixture of acetonitrile and 50 mM TEAA buffer (pH 7.4). The identity of the product was confirmed with LCMS. Sequences of trigger oligonucleotides, which are purely examples and should not be construed as limiting, are shown in Table 1. It should also be appreciated that use of DBCO-azide click reaction is only one example of a reaction that may be used to couple a trigger oligonucleotide to a nucleotide, analyte, or other element.

TABLE 1
Sequences of oligos for ddNTP

| | | SEQ |
|---|---|---|
| ddNTP | Oligo sequence | ID NO: |
| A | AAAGTCTAATCCGTCCCTGCCTCTATATCTCCACTC | 5 |
| U | GCATTCTTTCTTGAGGAGGGCAGCAAACGGGAAGAG | 6 |
| C | CACTTCATATCACTCACTCCCAATCTCTATCTACCC | 7 |
| G | CACATTTACAGACCTCAACCTACCTCCAACTCTCAC | 8 |

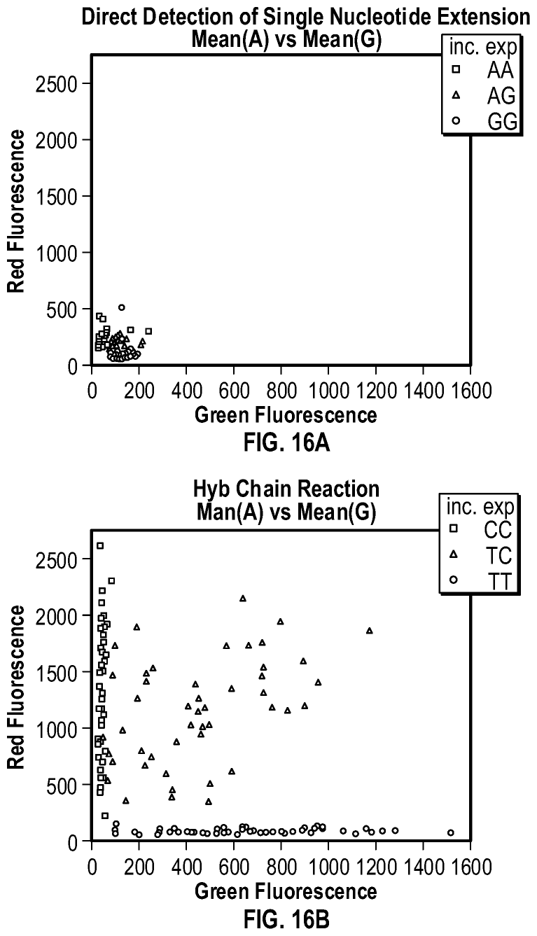

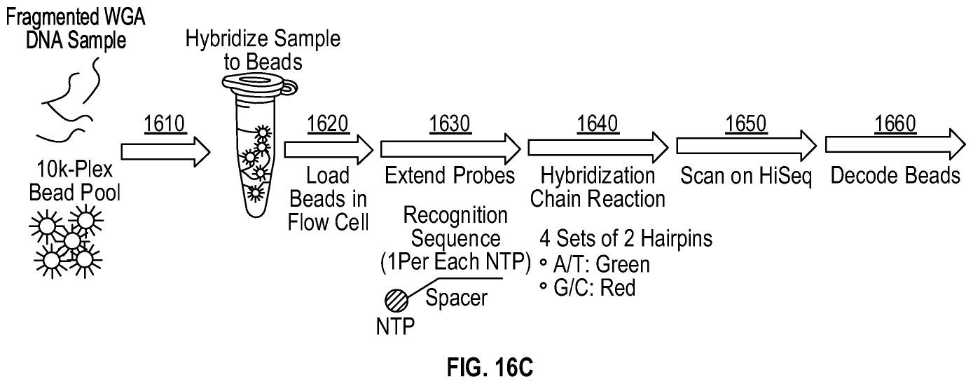

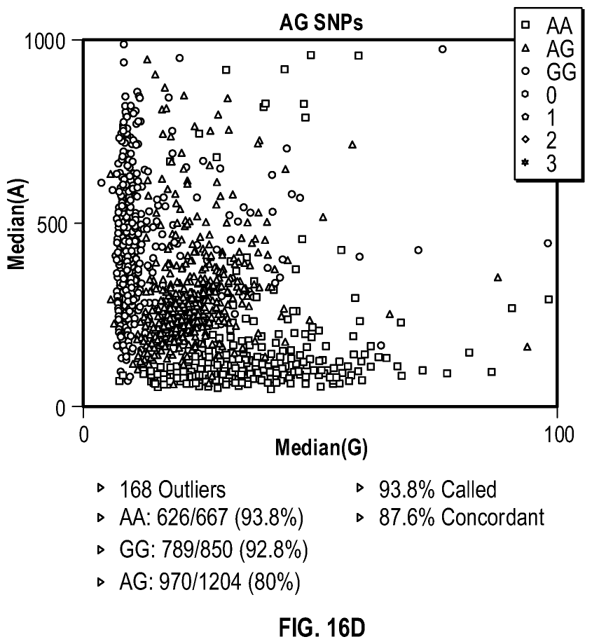

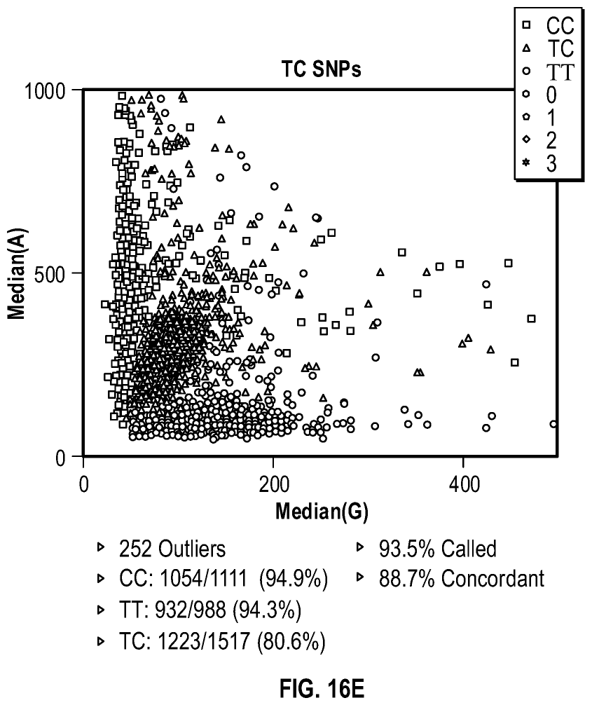

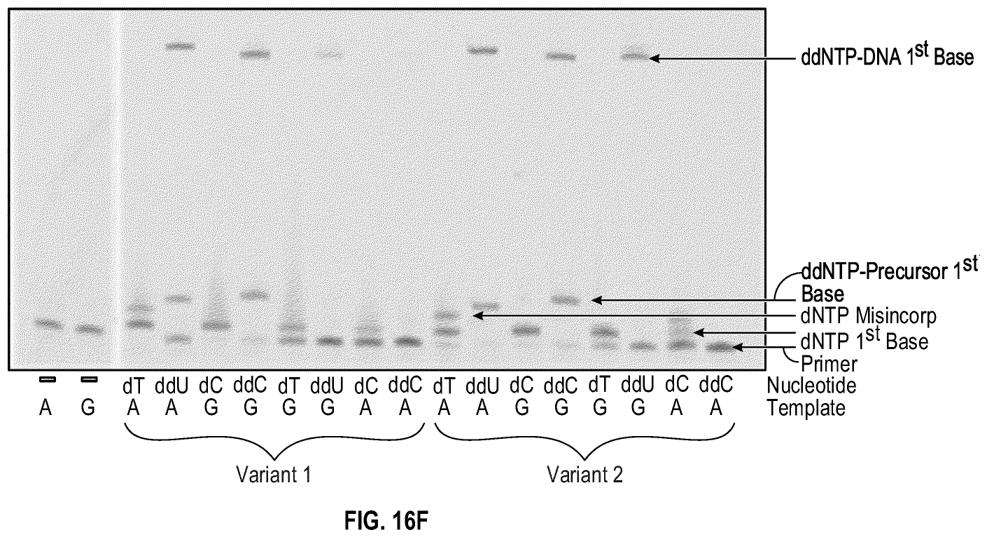

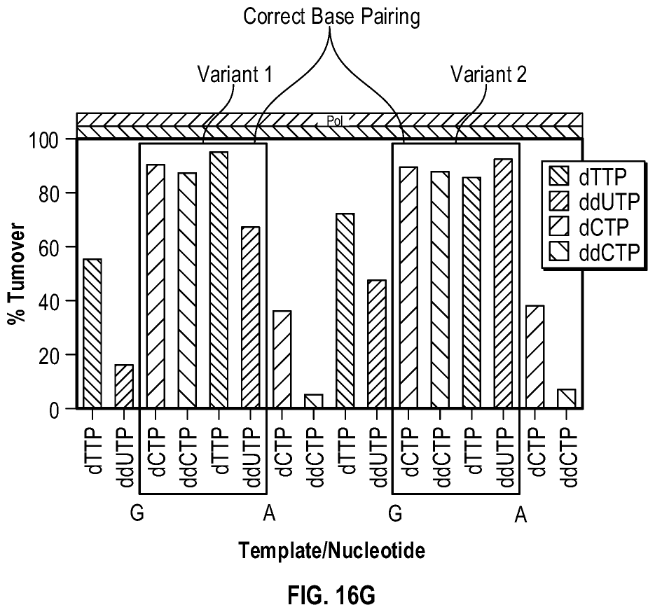

It was confirmed that SBS polymerases were able to incorporate the modified ddNTPs into a polynucleotide by extending a primer with the modified ddNTPs for a 5 minute incubation period at 37° C. in a solution of 1× ethanolamine at pH 9.9, 0.02% CHAPS, 9 mM MgSO₄, 1 uM polymerase, 200 nM P/T, and 10 uM dNTP/ddNTP. For example, FIG. 16F is a gel image showing a single base extension of a primer at the expected size (ddNTP-DNA 1^{st} base) for two variants of an SBS polymerase. FIG. 16G is a plot illustrating percent turnover of the ddNTPs, calculated via gel densitometry, is similar to that of their native counterparts.

As an initial proof of concept, the modified ddNTPs were employed in a genotyping assay that made use of beads with oligonucleotides similar to those described with reference to FIG. 1B. A pool of 332 different bead types loaded into a flow cell was tested. The oligonucleotide of each bead included a code that may be decoded using SBS chemistry to identify the bead, a spacer region to move the code far enough from the bead surface to avoid steric issues, a primer binding site, and a capture probe designed to capture a DNA analyte. More specifically, the DNA analytes were sequences for which single base extension of the capture probe with ffNs identified a SNP in a manner similar to that described with reference to FIG. 2A. Here, however, no separate sensing probe was used, and thus the single base extension was performed in a manner such as described with reference to FIG. 7D. In a first set of experiments, the single base extension was performed with nucleotides that were labeled with single fluorophores, more specifically ffG labeled with a single green fluorophore, and ffC labeled with a red fluorophore, and the fluorescence from the respective beads was measured. In a second set of experiments, the single base extension was performed with modified ddNTPs, more specifically ddUTP with a first trigger oligonucleotide “A”, and ddCTP with a second trigger oligonucleotide “B”. HCR using four different hairpins—one set A1 and A2 to add to trigger oligonucleotide A and labeled with red fluorophores, and one set B1 and B2 to add to trigger oligonucleotide B and labeled with green fluorophores, and the fluorescence from the respective beads was measured. FIG. 16A (direct detection of single nucleotide extension) is a plot illustrating measured red fluorescence and green fluorescence from the DNA analytes respectively labeled with single fluorophores (mean (A) vs. mean (G)), and FIG. 16B (hyb chain reaction) is a plot illustrating measured red fluorescence and green fluorescence from the DNA analytes respectively labeled with multiple fluorophores using HCR (mean (A) vs. mean (G). Each point is the average intensity from one of 332 different bead types included in the experiment. Comparing FIG. 16A to FIG. 16B demonstrates that compared to single fluorophore incorporation, HCR provides an average of 8 fold increase in intensity.

In an additional set of experiments, beads were hybridized to DNA analytes and genotyped on a sequencer. More specifically, FIG. 16C schematically illustrates an example process flow used to respectively label a plurality of DNA analytes with multiple fluorophores using HCR. A fragmented whole genome amplification (WGA) DNA sample was mixed in solution with a 10k-plex bead pool at process 1610, resulting in the sample being hybridized to the beads. The beads then were loaded into a flow cell at process 1620, and the probes extended by a single base, more specifically ffG labeled with a single green fluorophore, ffA labeled with a red fluorophore, ddUTP labeled with a first trigger oligonucleotide “A”, and ddCTP with a second trigger oligonucleotide “B”. One recognition sequence was provided per each NTP. At process 1640 HCR was performed using four different hairpins—one set A1 and A2 to add to trigger oligonucleotide A and labeled with red fluorophores, and one set B1 and B2 to add to trigger oligonucleotide B and labeled with green fluorophores, was performed and the fluorescence from the respective beads was measured. The beads were scanned on an Illumina HiSeq machine at process 1650, and the beads decoded at operation 1660. FIGS. 16D-16E are plots illustrating genotyping performance using at least the measured fluorescence from DNA analytes respectively labeled with multiple fluorophores using HCR. Each point is the average signal intensity in red and green channel from a single bead type. For each nucleotide incorporated, a different trigger and set of hairpins are used to generate signal. It may be understood from FIGS. 16D-16E that correct genotyping calls are maintained for the majority of bead types, while increasing signal and signal/background by approximately 8 fold.

Accordingly, it may be understood that the use of multiple fluorophores may significantly increase signal obtained from labeled elements. It will be appreciated that multiple fluorophores suitably may be coupled to any element, including but not limited to elements such as described herein.

### Other Examples

While various illustrative examples are described above, it will be apparent to one skilled in the art that various changes and modifications may be made therein without departing from the invention. The appended claims are intended to cover all such changes and modifications that fall within the true spirit and scope of the invention.

What is claimed is:

1. A method for detecting different analytes, the method comprising:
mixing different analytes with sensing probes, wherein at least some of the sensing probes are specific to respective ones of the analytes;
respectively capturing the analytes by the sensing probes that are specific to those analytes;
respectively coupling fluorophores to sensing probes that captured respective analytes;
mixing the sensing probes with beads, wherein the beads are specific to respective ones of the sensing probes, and wherein the beads include different codes identifying the analytes to which those sensing probes are specific;
respectively coupling the sensing probes to beads that are specific to those sensing probes;
identifying the beads that are coupled to the sensing probes that captured analytes using at least fluorescence from the fluorophores coupled to those sensing probes; and
identifying the analytes that are captured by the sensing probes coupled to the identified beads using at least the codes of those beads.

2. The method of claim 1, wherein each of the beads includes a first oligonucleotide having a sequence specific to one of the sensing probes, and wherein each of the sensing probes comprises a second oligonucleotide having a sequence that is complementary to the first oligonucleotide.

3. The method of claim 1 or claim 2, wherein the different codes comprise oligonucleotides having different sequences than one another.

4. The method of any one of claims 1 to 3, wherein at least one of the analytes comprises a nucleotide analyte.

5. The method of claim 4, wherein the sensing probe comprises an oligonucleotide sequence specific to hybridize to the nucleotide analyte.

6. The method of claim 4 or claim 5, wherein the nucleotide analyte comprises a DNA analyte.

7. The method of claim 4 or claim 5, wherein the nucleotide analyte comprises an RNA analyte.

8. The method of any one of claims 1 to 4, wherein at least one of the analytes comprises a non-nucleotide analyte.

9. The method of claim 8, wherein the non-nucleotide analyte comprises a protein.

10. The method of claim 8, wherein the non-nucleotide analyte comprises a metabolite.

11. The method of claim 8 or claim 9, wherein the sensing probe comprises an antibody selective to the non-nucleotide analyte.

12. The method of any one of claims 8 to 10, wherein the sensing probe comprises an aptamer selective to the non-nucleotide analyte.

13. The method of any one of claims 1 to 12, wherein the different analytes comprise a plurality of nucleotide analytes and a plurality of non-nucleotide analytes.

14. The method of any one of claims 1 to 13, wherein the fluorophores are coupled to the sensing probes after the analytes are captured by the sensing probes.

15. The method of any one of claims 1 to 14, wherein the fluorophores are coupled to the sensing probes before the sensing probes are coupled to the beads.

16. The method of any one of claims 1 to 14, wherein the fluorophores are coupled to the sensing probes after the sensing probes are coupled to the beads.

17. The method of any one of claims 1 to 16, wherein providing the fluorophores comprises coupling multiple fluorophores to the analytes.

18. The method of claim 17, wherein coupling multiple fluorophores to the analytes comprises using a hybridization chain reaction (HCR).

19. A system for detecting a plurality of different analytes, the system comprising:
sensing probes that are specific to respective ones of the different analytes;
beads that are specific to respective ones of the sensing probes and that include different codes respectively identifying the analytes to which those sensing probes are specific;
fluorophores to respectively couple to sensing probes that capture analytes; and
detection circuitry to identify beads that are coupled to the sensing probes that capture analytes, and to identify the analytes that are captured by the sensing probes coupled to those beads using at least the codes of those beads.

20. The system of claim 19, wherein each of the beads includes a first oligonucleotide having a sequence specific to one of the sensing probes, and wherein each of the sensing probes comprises a second oligonucleotide having a sequence that is complementary to the first oligonucleotide.

21. The system of claim 19 or claim 20, wherein the different codes comprise oligonucleotides having different sequences than one another.

22. The system of any one of claims 19 to 21, wherein at least one of the analytes comprises a nucleotide analyte.

23. The system of claim 22, wherein the sensing probe comprises an oligonucleotide sequence specific to hybridize to the nucleotide analyte.

24. The system of claim 22 or claim 23, wherein the nucleotide analyte comprises a DNA analyte.

25. The system of claim 22 or claim 23, wherein the nucleotide analyte comprises an RNA analyte.

26. The system of any one of claims 19 to 22, wherein at least one of the analytes comprises a non-nucleotide analyte.

27. The system of claim 26, wherein the non-nucleotide analyte comprises a protein.

28. The system of claim 26, wherein the non-nucleotide analyte comprises a metabolite.

29. The system of claim 26 or claim 27, wherein the sensing probe comprises an antibody selective to the non-nucleotide analyte.

30. The system of any one of claims 26 to 28, wherein the sensing probe comprises an aptamer selective to the non-nucleotide analyte.

31. The system of any one of claims 19 to 30, wherein the different analytes comprise a plurality of nucleotide analytes and a plurality of non-nucleotide analytes.

32. The system of any one of claims 19 to 31, wherein the fluorophores are coupled to the sensing probes after the analytes are captured by the sensing probes.

33. The system of any one of claims 19 to 32, wherein the fluorophores are coupled to the sensing probes before the sensing probes are coupled to the beads.

34. The system of any one of claims 19 to 32, wherein the fluorophores are coupled to the sensing probes after the sensing probes are coupled to the beads.

35. The system of any one of claims 19 to 34, wherein multiple fluorophores are coupled to the analytes.

36. The system of claim 35, wherein the multiple fluorophores are coupled to the analytes using a hybridization chain reaction (HCR).

37. A method for identifying target nucleic acids, comprising:
(a) hybridizing a plurality of probes to a plurality of nucleic acids comprising the target nucleic acids, wherein each probe comprises a 3′ end capable of hybridizing to a target nucleic acid and a 5′ end capable of hybridizing to a capture probe;
(b) extending the hybridized probes with a blocked nucleotide;
(c) removing the plurality of nucleic acids and non-extended probes from the extended probes; and
(d) hybridizing the extended probes to a plurality of capture probes immobilized on a surface.

38. The method of claim 37, wherein (a)-(c) are performed in solution.

39. The method of claim 37 or claim 38, further comprising repeating (a) and (b).

40. The method of any one of claims 37 to 39, wherein the blocked nucleotide comprises a detectable label.

41. The method of claim 40, wherein the label comprises a fluorophore.

42. The method of any one of claims 37 to 41, wherein (b) comprises polymerase extension.

43. The method of any one of claims 37 to 42, wherein (b) comprises ligase extension.

44. The method of any one of claims 37 to 43, wherein (c) comprises enzymatic degradation.

45. The method of any one of claims 37 to 44, wherein (c) comprises contacting the plurality of nucleic acids and the non-extended probes with a 3′ to 5′ exonuclease.

46. The method of claim 45, wherein the 3′ to 5′ exonuclease is selected from the group consisting of Exonuclease I, Thermolabile Exonuclease I, Exonuclease T, Exonuclease III, and Klenow I fragment.

47. The method of any one of claims 37 to 46, wherein the probes each comprise a 5′ end resistant to enzymatic degradation.

48. The method of claim 47, wherein the 5′ end resistant to enzymatic degradation comprises a phosphorothioate bond.

49. The method of claim 47 or claim 48, wherein (c) comprises contacting the plurality of nucleic acids with a 5′ to 3′ exonuclease.

50. The method of claim 49, wherein the 5′ to 3′ exonuclease is selected from the group consisting of RecJf, T7 Exonuclease, truncated Exonuclease VIII, Lambda Exonuclease, T5 Exonuclease, Exonuclease VII, Exonuclease V, and Nuclease BAL-31.

51. The method of any one of claims 37 to 50, wherein a plurality of beads comprise the surface.

52. The method of any one of claims 37 to 51, wherein the surface comprises a planar surface.

53. The method of any one of claims 37 to 52, wherein a flow cell comprises the surface.

54. The method of any one of claims 37 to 53, wherein (d) further comprises amplifying a signal from the hybridized extended probes.

55. The method of any one of claims 37 to 54, wherein (d) further comprises identifying the location of the hybridized extended probes on the surface.

56. The method of any one of claims 37 to 55, wherein the capture probes are different from each other.

57. The method of any one of claims 37 to 56, wherein the plurality of capture probes comprise a decoded array of capture probes.

58. The method of any one of claims 37 to 57, further comprising decoding the location of the capture probes on the surface.

59. The method of any one of claims 37 to 58, wherein the plurality of capture probes each comprise a primer binding site and a decode polynucleotide.

60. The method of claim 59, wherein decoding comprises: hybridizing a sequencing primer to the primer binding site, extending the hybridized primer, and identifying the decode polynucleotide.

61. The method of any one of claims 37 to 60, wherein the plurality of nucleic acids comprises genomic DNA.

62. The method of any one of claims 37 to 61, wherein the target nucleic acids comprise a single nucleotide polymorphism (SNP).

63. A system for identifying target nucleic acids, comprising:
an extension solution comprising:
a plurality of nucleic acids comprising the target nucleic acids,
a plurality of probes, wherein each probe comprises a 3′ end capable of hybridizing to a target nucleic acid and a 5′ end capable of hybridizing to a capture probe,
a plurality of blocked nucleotides,
an extension enzyme;
a degradation solution comprising a 3′ to 5′ exonuclease;
an array of capture probes immobilized on a surface; and
a detector to identify the location of an extended probe hybridized to a capture probe on the surface.

64. The system of claim 63, wherein a flow cell comprise the array of capture probes immobilized on a surface.

65. A system for identifying target nucleic acids, comprising:
a flow cell comprising a surface, an inlet for adding solutions to the surface, and an outlet for removing solutions from the surface, wherein an array of capture probes is immobilized on the surface;
an extension solution in contact with the inlet, the extension solution comprising:
a plurality of nucleic acids comprising the target nucleic acids,
a plurality of probes, wherein each probe comprises a 3′ end capable of hybridizing to a target nucleic acid and a 5′ end capable of hybridizing to a capture probe,
a plurality of blocked nucleotides,
an extension enzyme;
a degradation solution comprising a 3′ to 5′ exonuclease; and
a detector to identify the location of an extended probe hybridized to a capture probe on the surface.

66. The system of any one of claims 63 to 65, wherein the blocked nucleotide comprises a detectable label.

67. The system of claim 30, wherein the label comprises a fluorophore.

68. The system of any one of claims 63 to 67, wherein the extension enzyme comprises a polymerase.

69. The system of any one of claims 63 to 68, wherein the extension enzyme comprises a ligase.

70. The system of any one of claims 63 to 69, wherein the 3′ to 5′ exonuclease is selected from the group consisting of Exonuclease I, Thermolabile Exonuclease I, Exonuclease T, Exonuclease III, and Klenow I fragment.

71. The system of any one of claims 63 to 70, wherein the probes each comprise a 5′ end resistant to enzymatic degradation.

72. The system of claim 71, wherein the 5′ end resistant to enzymatic degradation comprises a phosphorothioate bond.

73. The system of claim 71 or claim 72, wherein the degradation solution further comprises a 5′ to 3′ exonuclease.

74. The system of claim 73, wherein the 5′ to 3′ exonuclease is selected from the group consisting of RecJf, T7 Exonuclease, truncated Exonuclease VIII, Lambda Exonuclease, T5 Exonuclease, Exonuclease VII, Exonuclease V, and Nuclease BAL-31.

75. The system of any one of claims 63 to 74, wherein the surface comprises a plurality of beads.

76. The system of any one of claims 63 to 75, wherein the capture probes are different from each other.

77. The system of any one of claims 63 to 76, wherein the plurality of capture probes comprise a decoded array of capture probes.

78. The system of any one of claims 63 to 77, wherein the plurality of capture probes each comprise a primer binding site and a decode polynucleotide.

79. The system of any one of claims 63 to 78, wherein the plurality of nucleic acids comprises genomic DNA.

80. The system of any one of claims 63 to 79, wherein the target nucleic acids comprise a single nucleotide polymorphism (SNP).

81. A method for detecting an element, the method comprising:
coupling an element to a substrate;
coupling a plurality of fluorophores to the element; and
detecting the element using at least fluorescence from the plurality of fluorophores.

82. The method of claim 81, wherein the element comprises an analyte.

83. The method of claim 82, wherein the analyte is coupled to a sensing probe.

84. The method of claim 83, wherein the analyte is coupled to the substrate via the sensing probe.

85. The method of claim 83 or claim 84, wherein the plurality of fluorophores is coupled to the element via the sensing probe.

86. The method of claim 83 or claim 84, wherein the plurality of fluorophores is coupled to the element via the substrate.

87. The method of any one of claims 81 to 86, wherein the plurality of fluorophores is coupled to the element before the element is coupled to the substrate.

88. The method of any one of claims 81 to 87, wherein the plurality of fluorophores is coupled to the element after the element is coupled to the substrate.

89. The method of any one of claims 81 to 88, wherein the substrate comprises a bead.

90. The method of any one of claims 81 to 89, wherein the plurality of fluorophores is coupled to the element using rolling circle amplification.

91. The method of claim 90, wherein the rolling circle amplification generates an elongated, repeated sequence, and wherein the plurality of fluorophores is coupled to respective, repeated portions of that sequence.

92. The method of claim 91, wherein the fluorophores are coupled to DNA intercalators, wherein the DNA intercalators couple to the elongated, repeated sequence.

93. The method of claim 91, wherein oligonucleotides comprising fluorophores and quenchers are hybridized to the repeated portions.

94. The method of any one of claims 81 to 89, wherein the element is coupled to a trigger oligonucleotide to which a plurality of fluorescently labeled hairpins self-assemble.

95. The method of any one of claims 81 to 89, wherein the element is coupled to a trigger oligonucleotide comprising a first trigger sequence A′ and a second trigger sequence B′, and wherein coupling the plurality of fluorophores to the element comprises contacting the trigger oligonucleotide with a plurality of first oligonucleotide hairpins and a plurality of second oligonucleotide hairpins,
wherein each of the first oligonucleotide hairpins includes a first fluorophore, a single-stranded toehold sequence A complementary to first trigger sequence A′, a first stem sequence B complementary to second trigger sequence B′, a second stem sequence B′ that is temporarily hybridized to first stem sequence B, and a single-stranded loop sequence C′ disposed between the first stem sequence B and the second stem sequence B′; and
wherein each of the second oligonucleotide hairpins comprises a second fluorophore, a single-stranded toehold sequence C complementary to single-stranded loop sequence C′, a first stem sequence B complementary to second trigger sequence B′, a second stem sequence B′ that is temporarily hybridized to first stem sequence B, and a single-stranded loop sequence A′ disposed between the first stem sequence B and the second stem sequence B′.

96. The method of claim 95, wherein responsive to hybridization of the single-stranded toehold sequence A of one of the first oligonucleotide hairpins to first trigger sequence A′ of the trigger oligonucleotide:
the second stem sequence B′ of that first oligonucleotide hairpin dehybridizes from the first stem sequence B of that first oligonucleotide hairpin;
the single-stranded toehold sequence C of one of the second oligonucleotide hairpins hybridizes to the single-stranded loop sequence of that first oligonucleotide hairpin; and
the second stem sequence B′ of that second oligonucleotide hairpin dehybridizes from the first stem sequence B of that second oligonucleotide hairpin.

97. The method of claim 96, wherein responsive to hybridization of the single-stranded toehold sequence A of another one of the first oligonucleotide hairpins to single-stranded loop sequence A′ of that second oligonucleotide hairpin:
the second stem sequence B′ of that first oligonucleotide hairpin dehybridizes from the first stem sequence B of that first oligonucleotide hairpin;
the single-stranded toehold sequence C of another one of the second oligonucleotide hairpins hybridizes to the single-stranded loop sequence of that first oligonucleotide hairpin; and
the second stem sequence B′ of that second oligonucleotide hairpin dehybridizes from the first stem sequence B of that second oligonucleotide hairpin.

98. The method of any one of claims 81 to 89, wherein the element is coupled to an oligonucleotide primer, and wherein coupling the plurality of fluorophores to the element comprises:
hybridizing an amplification template to the oligonucleotide primer; and
extending the oligonucleotide primer, using at least the amplification template, with a plurality of fluorescently labeled nucleotides to generate an extended strand comprising the plurality of fluorophores.

99. The method of claim 98, wherein at least one of the fluorophores is different than at least one other of the fluorophores.

100. The method of claim 98 or claim 99, further comprising dehybridizing the amplification template and forming the extended strand into a hairpin structure.

101. The method of any one of claims 81 to 89, wherein the element is coupled to an oligonucleotide primer, and wherein coupling the plurality of fluorophores to the element comprises:
hybridizing an amplification template to the oligonucleotide primer;
extending the oligonucleotide primer, using at least the amplification template, with a plurality of nucleotides that are respectively coupled to additional oligonucleotide primers;
hybridizing additional amplification templates to the additional nucleotide primers; and
extending the additional nucleotide primers, using at least the additional amplification templates, with a plurality of nucleotides that is either respectively coupled to fluorophores or is respectively coupled to further additional oligonucleotide primers.

102. The method of claim 101, further comprising hybridizing further additional amplification templates to the further nucleotide primers; and
extending the additional nucleotide primers, using at least the additional amplification templates, with a plurality of nucleotides that are either respectively coupled to fluorophores or are respectively coupled to still further additional oligonucleotide primers.

103. The method of any one of claims 81 to 89, wherein the element is coupled to a DNA origami comprising the plurality of fluorophores.

104. The method of claim 103, wherein the DNA origami comprises a combination of different fluorophores.

105. The method of claim 103 or claim 104, wherein the element is coupled to the DNA origami via copper(I)-catalyzed click reaction, strain-promoted azide-alkyne cycloaddition, hybridization of an oligonucleotide to a complementary oligonucleotide, biotin-streptavidin interaction, NTA-His-Tag interaction, or Spytag-Spycatcher interaction.

106. The method of any one of claims 81 to 89, wherein the element is coupled to an oligonucleotide, wherein the oligonucleotide comprises the plurality of fluorophores.

107. The method of claim 106, wherein the oligonucleotide comprises a hairpin.

108. The method of claim 106 or claim 107, wherein the oligonucleotide further comprises a radical scavenger.

109. The method of any one of claims 81 to 89, wherein the element is directly coupled to a first oligonucleotide, and the first oligonucleotide is hybridized to a second oligonucleotide that comprises the plurality of fluorophores.

110. A method for detecting a nucleotide, the method comprising:
adding the nucleotide to a first polynucleotide using at least a sequence of a second polynucleotide, wherein the added nucleotide includes a first moiety;
coupling a label to the added nucleotide by reacting the first moiety with a second moiety of the label, wherein the label comprises a plurality of fluorophores; and
detecting the added nucleotide using at least fluorescence from the plurality of fluorophores.

111. A method for detecting a nucleotide, the method comprising:
adding the nucleotide to a first polynucleotide using at least a sequence of a second polynucleotide, wherein the added nucleotide is coupled to a label comprising a plurality of fluorophores; and
detecting the added nucleotide using at least fluorescence from the plurality of fluorophores.

112. A method for detecting a nucleotide, the method comprising:
adding the nucleotide to a first polynucleotide using at least a sequence of a second polynucleotide, wherein the added nucleotide includes a first moiety;
coupling a label to the added nucleotide by reacting the first moiety with a second moiety of the label;
coupling multiple fluorophores to the coupled label; and
detecting the added nucleotide using at least fluorescence from the plurality of fluorophores.

113. A composition, comprising:
a substrate;
an oligonucleotide coupled to the substrate;
a nucleotide coupled to the oligonucleotide;
a moiety coupled to the nucleotide;
a label coupled to the moiety, wherein the label comprises a plurality of fluorophores; and
detection circuitry configured to detect the nucleotide using at least fluorescence from the plurality of fluorophores.
